(19) United States
(12) Patent Application Publication
(10) Pub. No.: US 2012/0329662 A1
(43) Pub. Date: Dec. 27, 2012

(54) HYPOXIA TUMOUR MARKERS

(76) Inventors: Catharine West, Manchester (GB); Crispin Miller, Mancherter (GB); Adrian Harris, Oxfordshire (GB); Francesca Buffa, Oxford (GB)

(21) Appl. No.: 13/517,411

(22) PCT Filed: Dec. 22, 2010
(86) PCT No.: PCT/EP10/70583

§ 371 Date: Aug. 29, 2012

(30) Foreign Application Priority Data
Dec. 22, 2009 (GB) 0922437.9

(51) Int. Cl. C12Q 1/68; C40B 40/06; C40B 30/00

(52) U.S. Cl. 506/7; 435/6.14; 435/6.12; 506/16

(57) ABSTRACT

The present invention relates to a method for assessing a hypoxia phenotype of a tumour of a subject in which the gene expression of between 3 and 50 hypoxia-related genes of a sample obtained from said tumour of the subject is determined, thereby obtaining a sample expression profile of said hypoxia-related genes. The sample gene expression profile is then compared with a reference expression profile of said hypoxia-related genes. The hypoxia-related genes comprise at least SLC2A1, VEGFA and PGAM1. Probes, arrays and kits for use in the method are also disclosed.

### FIELD OF THE INVENTION

The present invention relates to methods of assessing and classifying tumour characteristics, including tumour hypoxia phenotype, based on molecular markers, particularly gene expression of a compact hypoxia metagene, and to kits and related products for use in such methods.

### BACKGROUND TO THE INVENTION

Of the ˜300,000 patients who develop cancer within the UK each year, ˜50% will undergo radiotherapy at some point in their treatment. It has been estimated that a biologically-individualized approach to their treatment could improve outcome [1] with an estimated increase in survival rate of >10% [2]. Attempts to find a reliable predictor of radioresponse highlighted the importance of tumour radiosensitivity, proliferation and hypoxia, but no method has proved logistically feasible to integrate within routine clinical practice. Research in this area is now progressing to exploit the new genomic technologies. Molecular array profiling to improve current approaches to predict chemo/radiotherapy outcomes was identified as a priority research area by the 2003 NCRI Radiotherapy and Related Radiobiology Progress Review.

Hypoxia is a common feature of solid tumours. It arises when tissue oxygen demands exceed the oxygen supply from the vasculature. Hypoxic regions develop within solid tumours due to aberrant blood vessel formation, fluctuations in blood flow and increasing oxygen demands from rapid tumour expansion. Hypoxia is known to be highly heterogeneous within tumours in terms of its spatial distribution, severity and kinetics. Hypoxia arises through different mechanisms associated primarily with limits in oxygen diffusion (chronic hypoxia) and blood perfusion (acute hypoxia). In addition, hypoxia regulates several different cellular pathways that have unique activation kinetics and sensitivity to oxygen concentration. As a consequence, hypoxia regulated gene expression is complex and displays large temporal characteristics.

Hypoxia is the result of an imbalance between oxygen delivery and oxygen consumption resulting in the reduction of oxygen tension below the normal level for a specific tissue [3]. Using Eppendorf histography electrodes, oxygen tensions were measured in several cancer types showing a range of values between 0 and 20 mmHg in the tumour tissues, which were significantly lower than those of the adjacent tissue (24-66 mmHg) [4, 5, 6]. Oxygen tensions measured in breast cancers of stages T1b-T4 revealed a median pO₂ of 28 mmHg compared with 65 mmHg in normal breast tissue [7]. Hypoxia occurs in many disease processes, and it is widespread in solid tumours due to the tumour outgrowing the existing vasculature.

This may result in the death of cancer cells if it is severe and prolonged. In vivo different conditions have been recognised. Chronic or diffusion-limited hypoxia is due to a concentration gradient of diffusion, about 150-200 μM, due to the metabolism of oxygen as it diffuses further away from capillaries and will also be related to the metabolic activity of the tumour. Acute hypoxia is a transient perfusion-limited state, which occurs when an aberrant blood vessel is temporarily shut off, so that the cells adjacent to the capillaries die because of the insufficient blood supply. Intermittent hypoxia occurs when blood vessels are reopened and the hypoxic tissue is reperfused with oxygenated blood, leading to an increase in the levels of reactive oxygen species and resulting in the tissue damage as a result of hypoxia-reoxygenation injury [8]. The recent findings suggest that intermittent hypoxia might protect endothelial cells through a stronger stabilisation of hypoxia-inducible factor-1 (HIF-1) compared with chronic hypoxia [8].

In addition to mild hypoxia (0.01-2% O₂), some tumours contain regions of severe hypoxia (<0.01% O₂) called anoxia. This is a functionally different state to hypoxia and leads to coordinated cytoprotective programmes known as the unfolded protein response and integrated stress response, which are critical for tumour survival [9].

In hypoxic conditions, numerous cellular mechanisms are compromised and an adaptive response occurs which allows cancer cells to adapt to this hostile environment. This renders them more resistant and ability to survive and even proliferate, promoting tumour development [10].

The Adaptive Response to Hypoxia

The cellular response to hypoxia is modulated by the ubiquitous family of transcription factors known as hypoxia-inducible factors consisting of αβ-heterodimers, which include HIF-1α, HIF-2α, HIF-3α and HIF-1α. The HIF-1α subunit is the most ubiquitously expressed and acts as the master regulator of oxygen homeostasis in many types of cells. In the presence of oxygen, the von Hippel-Lindau tumour suppressor (pVHL), which is the recognition component of an E3 ubiquitin ligase complex, targets HIF-1α protein which is degraded within minutes by the ubiquitin-proteasome pathway. The interaction of pVHL and HIF-1α requires the hydroxylation of two proline residues, at positions 402 and 564 catalysed by prolyl-hydroxylases. Three prolyl-hydroxylase domain (PHD) enzymes, known as PHD1, PHD2 and PHD3, were identified in mammalian cells and were shown to hydroxylate HIF-1α although at varying levels of activity. In hypoxia, the proline residues are not hydroxylated and thus HIF-1α is stabilised and translocated to the nucleus where, with the recruitment of a number of cofactors including p300, it is dimerised with HIF-1α. The HIF-1 heterodimer targets hypoxia-responsive elements containing genes encoding essential pathways in systemic, local and intracellular homeostasis, providing the essential compensatory mechanism to increase the delivery of oxygen and nutrients while removing the waste products of metabolism [8, 10-13].

Hydroxylase activity is iron and ascorbate dependent. The recent studies found that physiological concentrations of ascorbate (25 μM) strongly suppress HIF-1α protein levels and HIF transcriptional target. Similar results were observed with iron supplementation [14].

The factor inhibiting HIF-1 (FIH-1) is another dioxygenase, which hydroxylates a conserved asparagine residue Asn803 within the C-terminal transactivation domain (TAD) under normoxic condition, acting synergistically with the PHD system to block the transcriptional activity of HIF-1α. Recently, it was shown that the cytoplasmic location of FIH-1 in invasive breast cancer is associated with an enhanced hypoxic response and a worse prognosis [15].

Two different expression patterns of immunohistochemical staining for HIF-1α have been described in primary tumour samples. One depends on the distance from blood vessels associated with a decreased oxygen concentration. The other expression pattern is diffuse throughout the entire tumour, indicating that HIF-1α can be triggered by factors other than hypoxia [16]. Growth factors (e.g. IGF2, TGFα, IGF1R and EGFR), cytokines and other signalling molecules stimulate HIF-1α synthesis via activation of the phosphatidylinositol 3-kinase (PI3K) or mitogen-activated protein kinase (MAPK) pathways in a cell-type-specific manner. PI3K mediates its effects through its target AKT and the downstream kinase mTOR (mammalian target of rapamycin which is inhibited by rapamycin, a macrolid antibiotic), which have a regulating role in protein synthesis. Stimulation of the human breast cancer cell line MCF-7 with heregulin activates the human epidermal growth factor receptor 2 (HER)/Neu receptor tyrosine kinase, and results in an increased HIF-1α protein synthesis, dependent upon activity of PI3K, AKT and mTOR. Oncogenes (e.g. v-Scr and H-Ras) induce constitutive expression of HIF-1α. The signalling pathway mediated by wingless-type (Wnt) proteins is implicated at several stages of mammary gland growth and differentiation, and the recent evidences suggest a role in breast carcinogenesis [17]. Wnt/βcatenin pathway is involved in the epithelial-mesenchymal transition (EMT), a crucial process in tumour development, increasing tumour cells proliferation, migration and invasion [18, 19]. Although the process has not been well elucidated, the possibility that HIF-1 induces tumour cells to undergo EMT has been demonstrated in colon cancer [20] and prostate cancer [21], and the recent data indicate that the Wnt/βcatenin signalling pathway may be critical in the signal of HIF-1α for inducing prostate cancer cell to undergo EMT22. Genetic abnormalities observed frequently in human cancers, including loss-of-function mutations (e.g. VHL, p53 and PTEN), are also associated with increased expression of HIF-1α and HIF-1 inducible genes [23-25].

In microenvironments, where oxygen is scarce and glucose consumption is high, a metabolic shift from oxidative to glycolytic metabolism occurs. The important role of the family of glucose transporters (GLUT-1 and GLUT-3 being hypoxia-inducible) has been extensively investigated in cancer cell lines and surgical specimens [26]. However, while HIF-1 stimulates glycolysis, it also actively downregulates mitochondrial function and oxygen consumption by inducing pyruvate dehydrogenase kinase 1 (PDK1), which phosphorylates and inactivates pyruvate dehydrogenase (PDH), the mitochondrial enzyme that converts pyruvate into acetyl-CoA. HIF-1 also induces the expression of genes encoding lactate dehydrogenase A (LDHA), which converts pyruvate into lactate, and cytochrome c oxidase subunit COX4-2, which replaces COX4-1 and increases the efficiency of mitochondrial respiration under hypoxia. These events result in a drop in mitochondrial oxygen consumption and reduced free radical generation, thereby decreasing cell death in response to hypoxia [27-29].

A well-defined link between the upregulation of HIF-1 in hypoxia and the maintenance of pH balance is a group of genes that encode for transmembrane carbonic anhydrases (CAs). CAs have been described in a variety of tumour types, including breast cancer, where its expression increases with increasing distance from blood vessels and decreasing oxygen concentration, and is extreme in perinecrotic areas [30-32].

Hypoxia also plays a crucial role in modulation of tumour angiogenesis that is required for tumour growth and metastasis [33, 34]. The most characterised HIF-regulated gene is vascular endothelial growth factor (VEGF), which is involved in regulating endothelial cell proliferation and blood vessel formation in both normal and cancer cells35. Other than VEGF (or VEGF-A), the predominant factor that influences angiogenesis, its family includes VEGF-C, D, E and placental growth factor (PLGF). Alternative splicing of VEGF-A forms four isoforms including VEGF121, VEGF165, VEGF189 and VEGF206 [36]. However, the recent studies suggested a HIF-1-independent mechanism that regulates pro-angiogenic activity of VEGF by showing induction of tumour angiogenesis before the activation of HIF-1[37].

Activation of nuclear factor-kB (NF-KB) under hypoxia was identified, which may enhance its role in oncogenic signalling pathways, apoptosis and cell adhesion. A role of NF-kB in TNFα-mediated HIF-1 accumulation by hypoxia-independent mechanisms was described [38]. The recent studies have further suggested an important link between hypoxia and the notch-signalling pathway, a cell-cell communication mechanism closely associated with cell differentiation [39].

From a clinical point of view, hypoxia is a potential therapeutic problem as the adaptive changes in response to hypoxia lead towards treatment resistance to both radio- and chemotherapy. An additional physical effect of hypoxia, which was recognised 50 years before HIF was discovered, relates to oxygen free radicals. It has been recognised for many years that the oxygenation status of a tumour is an important factor affecting the cytotoxicity of radiation, and it has become well established that cells in oxygen-deficient areas may cause solid tumours to become radioresistant. This phenomenon is known as ‘hypoxic radioresistance’, and is the result of a lack of oxygen in the radiochemical process by which ionising radiation is known to interact with cells. The phenomenon is most clearly seen after large single doses of radiation, but also exists in normal fractionated radiotherapy [40]. Hypoxia also directly induces resistance of solid tumours to chemotherapy by reducing the generation of free radicals by agents such as bleomycin and doxorubicin, and by the inhibition of cell cycle progression and proliferation, since a number of drugs specifically target highly proliferating cells [41, 42]. The oxygen level is an important factor in the action of many antineoplastic agents, several of which have been classified in vitro and in vivo by their selective cytotoxicity towards oxygenated and hypoxic tumour cells in animal models.

Current Methods for Measuring Hypoxia

There are many possible ways for assessing the level of hypoxia in tumours. The main direct approach is to measure intratumoural pO₂ with polarographic electrodes [43]. Oxygen electrode measurements are often referred to as the gold standard, but the approach is limited to accessible tumours. Hypoxia-specific markers, such as pimonidazole and EFS, are of interest but require pre-biopsy administration of drug. PET and cross-sectional imaging methods are also being investigated, but can only be assessed prospectively and are currently difficult to perform within a multicentre, phase III setting.

Indirect techniques being explored include measuring the immunohistochemical expression of hypoxia-regulated proteins, such as carbonic anhydrase 9 (CA9) and HIF-1α [44, 45]. High expression of HIF-1a and CA9 is associated with adverse prognosis in several cancers including HNSCC [44, 46]. Although high expression of HIF-1α and CA9 was thought to reflect the hypoxic nature of a tumour and activation of the HIF pathway, other studies reported no association with survival [47, 48] or association for only one factor [49]. Some of these anomalous findings have been explained by the different half-lives for CA9 (days) and HIF-1α (minutes) proteins [50]. It is more probable that, because hypoxia influences many biological pathways, a single factor is incapable of adequately describing this complex response.

The use of the strongly hypoxia-inducible genes such as CA9 [51] and HIF-1α [52] as surrogate markers of hypoxia is attractive because the method is feasible to explore retrospectively using formalin-fixed, paraffin-embedded (FFPE) material. However, although the approach is suitable for routine use, it is limited because of variability in marker expression within and between tumours, and lack of hypoxia specificity.

More recently microRNA (miRNA) expression alterations have been described in cancer. miRNAs are non-coding RNA oligonucleotides that have emerged as important regulators of gene expression including hypoxia. hsa-miR-210 overexpression is induced by hypoxia and its expression levels in breast cancer samples are an independent prognostic factor [53]. hsa-miR-210 appears to regulate a gene programme that does not overlap with that regulated directly by HIF53. The use of miRNA expression to assess tumour hypoxia is a developing area of research that requires further study.

However with RNA expression microarrays, it is now possible to monitor the expression of several tens of thousands of genes at once. In oncology, this ability is exploited to extract lists of genes (or gene signatures) rather than to rely on a few clinical variables for diagnosis [54, 55] or prognosis. For the latter, these gene sets include those derived from clinical data, in which correlation with a supervised classifier identifies the clinical group with a better or worse prognosis [56, 57, 58]. More recently, in vitro derived gene sets have been described containing genes associated with a particular phenotype hypothesized to be clinically important [59, 60, 61, 62]. This allows an unbiased test of such a hypothesis, by applying the in vitro derived signature to a separate patient microarray study. This latter type of study recently demonstrated that a gene signature for hypoxia could act as a prognostic factor in a range of different tumour types. In this latter study, Chi et al. [61] also measured the temporal gene expression programs under hypoxia for several primary cell lines in vitro. The Chi et al. dataset might be used to extract hypoxic gene signatures that reflect differences between slow and fast hypoxia kinetic responses and their contribution to prognosis because of the large dependency of hypoxic gene expression on time. In view of the above, it is apparent that there exists a need for improved hypoxic gene signatures for the identification, diagnosis, and treatment of cancer.

Towards this goal, we recently developed a hypoxia-associated gene signature [63]. Fifty-nine H&N tumours were profiled using Affymetrix U133plus2 GeneChips and a signature derived by clustering around the in vivo expression of well-known hypoxia-associated genes. Strongly correlated up-regulated genes defined a signature comprising 99 genes. The median expression of the 99 genes was an independent prognostic factor for recurrence-free survival in a publicly available H&N cancer data set [64], outperforming the original intrinsic classifier. In a published breast cancer series [65], the hypoxia signature was a significant prognostic factor for overall survival independent of clinicopathologic risk factors and a trained profile. This work highlights the validity of using a multiplex hypoxia biomarker. Although the 99-gene signature was prognostic for treatment outcome in different tumours, to be of use clinically it is important to show it can predict for benefit from hypoxia-modifying therapy.

Head & Neck Cancer

In 2008, head and neck cancers accounted for approximately 4% to 5% of all the malignant disease in the United States [66]. Head and neck squamous cell carcinoma (HNSCC) comprises the vast majority of head and neck cancer (HNC). Surgery, radiotherapy, and chemotherapy play a role in the management of the disease, and 5-year survival rates for patients with advanced cancers are ˜50% [67, 68]. Many factors contribute to this poor prognosis, including late presentation of disease, nodal metastases, and the failure of advanced cancers to respond to conventional treatments [69].

Breast Cancer

Breast cancer is the most commonly occurring malignancy in women, and is responsible for approximately 500,000 deaths per year worldwide. In the recent years, the encouraging trend towards earlier detection and the increasing use of systemic adjuvant treatment have improved the survival rates, but still nearly half of the breast cancer patients treated for localised disease develop metastases.

Tumour Hypoxia—Prognostic in Head and Neck Cancer and Breast Cancer

Tumour hypoxia is an independent adverse prognostic factor in many tumours, including HNSCC and breast cancer [43, 10]. Evidence showing that hypoxia is important in tumour progression [70] and prognosis [10] has spurred research into developing therapies that target hypoxic cells. Therapeutic strategies include modification of the hypoxic environment or targeting components of the HIF-1 signalling pathway [71, 72]. Although these approaches have shown some promising results, it remains difficult to identify hypoxic tumours and those patients most likely to benefit from hypoxia modification therapy.

Various methods have been developed to measure tumour hypoxia directly or indirectly, including imaging by blood oxygen level-dependent magnetic resonance (BOLD MRI), hypoxia-activated scanning agents (e.g. nitroimidazoles, fluoromisonidazole) and immunohistochemical analysis for hypoxia-induced genes. Currently, the Eppendorf polarographic oxygen electrode is the rarely used method considered the ‘gold standard’, but it correlates poorly with other markers [73, 74]. However, all these techniques have limitations due to their invasiveness or necessity for pre-injection of a non-approved agent (e.g. pimonidazole), or lack of approved imaging agents [75, 76].

In other types of cancers, this technique has generated many correlations between hypoxia and cancer treatment and outcome [77]. For this reason, efforts have been encouraged to non-invasively detect and localise regions of poor oxygenation in tumours. The recent studies suggested that hypoxia-regulated genes could be used alternatively as endogenous hypoxia markers, which are strongly related to aggressive disease and poor prognosis [78]. Although HIF-1α expression may also be influenced by other pathways, a significant correlation between oxygen tension and HIF-1α has been reported in cervical cancer, suggesting that HIF-1α might be used as a surrogate for tumour hypoxia [78]. Elevated HIF-1α protein levels are observed in the majority of human cancers and are associated with advanced tumour grade, increased angiogenesis, resistance to chemotherapy and radiotherapy, and increased patient mortality [79, 81]. Similarly, increased HIF-1αprotein levels have been reported in HNSCC tissues with poor disease prognosis [45, 46, 79, 80]. By using HIF-1α as a marker for hypoxia, approximately 25-40% of all invasive breast cancer samples are hypoxic; the frequency of HIF-1α-positive cells increases in parallel with increasing pathologic stage and is associated with a poor prognosis. In a recent study, Generali et al. showed that in the human breast cancer HIF-1α expression is also a predictive marker of chemotherapy failure, with a significant inverse correlation between pre-treatment levels of HIF-1α and disease response [82]. In addition, they found that HIF-1α is upregulated in patients with higher risk of relapse, identifying ER positive patients with a poor outcome, similar to that of ER negative patients. Dales et al. investigated HIF-1α in 745 breast cancer samples using immunohistochemical assays on frozen sections and observed that high HIF-1α expression was associated with poor overall survival and high metastasis risk.

This was in node-negative and node-positive patients [83]. HIF-1α was found to be an indicator of poor prognosis in both node-negative and node-positive breast cancer [84, 85].

In several studies, downstream targets of HIF-1α were considered as hypoxia markers. Expression of CAIX is localised to the perinecrotic area of tumours and has been observed to start at a median distance of 80 μM from a blood vessel, where the oxygen tension drops to 1% or less [86]. Previous studies showed that CAIX is a marker in tumour samples and that its expression was associated with poor prognosis, independently of the other commonly recognised prognostic parameters. However, using a primary chemo-endocrine setting of therapy, Generali et al. showed that CAIX expression was significantly associated with poor disease-free survival (DFS) and overall survival (OS) but failed to be an independent predictor of DFS in multivariate analysis, although they suggested a contribution of CAIX expression to tamoxifen resistance [31]. Other authors found that CAIX was rarely expressed in normal epithelium and benign lesions, but present in a significant percentage of ductal carcinoma in situ (DCIS) and invasive breast carcinoma. Loss of CAXII and/or gain of CAIX expression may be associated with a high risk of progression, and thus may be of prognostic significance [87]. Recently, Brennan et al. studied CAIX in premenopausal breast cancer patients and reported that CAIX was an independent prognostic parameter in lymph node-positive patients [88].

Many studies have confirmed the clinical relevance of VEGF expression as a significant and independent prognostic variable for relapse-free and overall survival [89-92]. The recent studies observed that HER-2/neu receptors play an important role in heregulin-induced angiogenesis [93, 94]. In addition, many studies have suggested that microvessel density (MVD), a surrogate marker of tumoural angiogenesis, is correlated with poor prognosis invasive breast cancer [34]. However, measurements of MVD are poorly reproducible [95] and standardised methods will be needed for MVD assessment [96, 97].

Gene Profiling Head and Neck and Breast Cancer for Hypoxia: Towards Personalised Therapy

Understanding the association between biological factors and treatment response is important in order to identify patients, who will derive benefit from certain therapeutic regimens. This would enable the design of management plans optimised for the individual patient. The recognition of prognostic and predictive markers is also crucial to identify novel targets for specific therapeutics.

As microarray techniques allow the analysis of thousands of expressed genes, this should be a promising approach for identifying multiple factors acting in concert to influence outcome and response to therapy.

Although hypoxia has been recognised as an important determinant of clinical outcomes in human cancers, it has been difficult to define tumour phenotypes based on hypoxia responses. Recently, Winter et al. [98] assessed the mRNA profile of head and neck cancer (HNSCC) samples defining an in vivo hypoxia metagene by clustering around the RNA expression of a set of well-known hypoxia-regulated genes (e.g. CAIX, GLUT1 and VEGF). The metagene contained many previously described in vitro-derived hypoxia response genes, and was prognostic for treatment outcome in independent data sets including breast cancer [98].

Chi et al., using DNA microarrays, found that in breast cancer samples the expression of most of the genes in the hypoxia response signature varied, and were separated into two groups by hierarchical clustering based on the level of hypoxia response. All the normal breast samples and fibroadenomas were clustered in a group characterised by low expression of the hypoxia signature, while ductal adenocarcinoma samples were split between low and high hypoxia response groups. In this way, the authors were able to stratify human cancers according to the presence and amplitude of a hypoxia response and showed that breast cancer tumours with a strong gene expression signature of the hypoxia response had a significantly worse prognosis and correlated with cancer progression and metastasis [61].

Seigneuric et al. focused their attention on the time dependency of hypoxia-regulated genes expression, and described how the early and the late hypoxia responses are very different at the transcriptional level. Using published data from the microarray data of Chi et al., they showed that survival differences are correlated with early hypoxia signatures, but not late hypoxia responses [99].

This evidence suggests that treatment response and outcomes come to depend on individual genetic features. The identification of molecular biomarkers with the potential to predict treatment response outcome is essential for selecting patients to receive the most beneficial therapy, and it might drive stratification in clinical trials. Hypoxia is a key physiological difference interacting independently with many key pathways, and will need to be incorporated into the algorithms used. Examples of drugs already developed particularly relate to VEGF blockade, but many signal transduction blockers targeting HER2 and EGFR will also inhibit hypoxia signalling. Many enzymes and signalling pathways described above are targets for drugs in phase I trials and for cost effectiveness we need to understand the biology to select appropriate patients.

A recent study exploring gene expression profiling to predict H&N cancer patient outcome following chemoradiotherapy highlighted the lack of transferability of signatures [100]. Previously published signatures for radiosensitivity, hypoxia and proliferation were not significantly correlated with outcome. Ein-Dor et al [101] highlighted the lack of overlap between expression profiles that are prognostic for cancer treatment outcome and showed that many equally prognostic gene lists could be produced from the van't Veer breast cancer signature. It was suggested that this is due in part to the many genes that correlate with survival. However, Shen et al [102] analysed four independent microarray studies to derive an inter-study validated meta-signature associated with breast cancer prognosis, which was comparable or better at providing prognostic information compared with the intrinsic signatures. It may be, therefore, that the best (most stable) hypoxia-associated gene signature/meta-signature is yet to be derived.

Patient Stratification For Hypoxia Targeted Therapy (Radiotherapy/Chemotherapy)

There is considerable evidence that hypoxia limits tumour cell response to radiation and chemotherapy and predisposes them to metastasis [43]. There is also evidence from three independent trials that hypoxic tumours gain the greatest benefit from hypoxia-modifying therapy. The first study showed the level of pimonidazole (a hypoxia marker) binding in head & neck (H&N) tumours predicted likely benefit from hypoxia-modifying ARCON—accelerated radiotherapy plus carbogen and nicotinamide—with survival rates of ˜60% and ˜18% for hypoxic tumours receiving ARCON vs conventional radiotherapy, respectively [103, 104]. The second study was linked to a phase III H&N cancer trial (DAHANCA 5), which showed addition of hypoxia-modifying nimorazole to conventional radiotherapy was associated with an increase in locoregional control (49% vs 33%) and overall survival (26% vs 16%) [105]. Patients in the DAHANCA 5 trial with high plasma osteopontin levels (associated with tumour hypoxia) were most likely to benefit from nimorazole. Disease-specific survival rates were 51% and 21% for patients with high osteopontin levels undergoing hypoxia-modifying vs radiotherapy alone [106]. A third study showed patients with hypoxic tumours identified using 18F-FMISO PET had an improved outcome following chemoradiotherapy plus the bioreductive agent tirapazamine compared with hypoxic tumours that received chemoradiotherapy alone (100% vs 39% locoregional control rate) [107]. These three studies highlight the potential to increase the individualisation of cancer treatment by using hypoxia-modifying therapy but there is an unmet need for a validated and qualified biomarker of hypoxia. Numerous approaches are being investigated and the work carried out to date clearly shows that the aim is scientifically justified [103, 106, 107].

However, an FDA approved biomarker has yet to be developed under Good Clinical Laboratory Practice (GCLP) conditions for use in the individualization of cancer patient treatment. The lack of introduction of hypoxia-modifying approaches into clinical practice in the UK and elsewhere, despite evidence for therapeutic benefit, is generally because there is no commercialised biomarker for selecting patients most likely to benefit. There is currently considerable interest in combining molecularly targeted agents with radiotherapy to improve cancer patient outcome. This important avenue of research will not supersede the need for a hypoxia biomarker as some of the new drugs being developed target hypoxia pathways. Given the huge health burden from cancer in the UK, the development of a validated and qualified hypoxia biomarker is an important area of research.

The Exploitation of Tumour Hypoxia for Therapeutic Benefit

Despite being strongly linked to the poor response of cancer patients to standard treatments, low levels of oxygen, the presence of necrosis and HIF-1 expression are unique features of solid tumours. They do not occur in normal tissues under normal physiological conditions and so are potentially exploitable.

Increased vascular leakage from immature tumoural vasculatures can result in increased interstitial blood pressure, thereby, worsening tumour hypoxia and impeding effective drug delivery to the tumour. Jain et al. popularized the concept of normalization of tumour vasculature through antiangiogenic therapy such as bevacizumab [108]. This concept was supported by clinical data in colorectal cancers, where treatment with bevacizumab was shown to reduce tumour interstitial pressure [109].

Another promising approach to overcoming tumour hypoxia in HNSCC is the combined use of the nicotinamide vasodilator and carbogen breathing (ARCON) to increase the oxygen partial pressure of tumours. ARCON (Accelerated Radiotherapy with CarbOgen and Nicotinamide) has produced a 3-year local control rate in excess of 80% for advanced stage T3-4 laryngeal and oropharyngeal cancers [104]. Presently, a phase III clinical trial testing the efficacy of ARCON in laryngeal cancers is ongoing in Europe [104].

A promising strategy to exploit tumour hypoxia is through agents that have high selectivity for killing hypoxic cells, the first drug of which is tirapazamine (TPZ or SR4233). In a randomized phase II trial, the combination of TPZ, cisplatin and RT was found to be better than 5FU, cisplatin and RT110. In contrast, we found that the addition of TPZ to an aggressive regimen of induction and concurrent cisplatin and 5FU with RT did not result in improved outcomes in a small randomized phase II study [111]. A phase III trial testing the benefit of adding TPZ to concurrent RT and cisplatin has been completed and the results are pending.

TPZ, however, does have several limitations; these include the poor diffusion of TPZ through hypoxic tissue and its requirement of less stringent hypoxia for activation, that can result in normal tissue toxicity in poorly oxygenated organs. There are therefore strong interests in developing novel hypoxic cell cytotoxins with more specific antitumour activity.

Dinitrobenzamide mustards (DNBMs) are a new and highly potent class of hypoxic cytotoxins discovered by the Auckland University group. These compounds have improved properties over TPZ; including a more stringent requirement for hypoxia for activation and a substantial bystander killing effect.

Hypoxia-Targeted Gene Therapy

Hypoxic cells can be targeted using gene therapy. This is achieved by using hypoxia and the switch on of HIF transcriptional activity as the trigger for therapeutic gene expression. Most hypoxia-targeted gene therapies utilize promoters containing HRE enhancer response elements. The HRE/HIF-1 regulation system is common to all mammalian cells and human tissues tested, and the HIF-1 subunit is overexpressed in 68-84% of the tumour types analysed [112]. Further, hypoxia and HIF-1 are not limited to primary cancers but are detectable in disseminated micrometastases [113, 114]. Therefore HRE-mediated gene therapy should be applicable to a wide range of cancers. The HRE promoters have also been reported to be “dual” responsive to both hypoxia and radiation potentially increasing therapeutic gene expression in combined hypoxia-targeted gene therapy and radiotherapy protocols [115]. Hypoxia responsive promoters have mainly focused on the use of HREs combined with a minimal viral promoter. Dachs et al 1997 [116] first demonstrated the potential utility of a HRE-driven gene therapy approach. A trimer of the HRE from murine PGK was used to hypoxically regulate expression of the bacterial enzyme cytosine deaminase (CD) and sensitize tumour cells to 5-fluorouracil (5-FU). Since this first demonstration the PGK HRE [116, 117, 118] and those from VEGF [119, 120], EPO [121, 122] and LDH [123] have been used extensively in gene therapies. They have been used to drive tumour specific expression of prodrug activating enzymes [116, 122, 123, 124], pro-apoptotic proteins and anti-tumour cytokines [126], and, more recently, to drive tumour-specific viral replication and oncolysis [127, 128].

Hypoxia-Targeted Chemotherapy

The potential to target tumours using hypoxia-selective chemotherapy drugs has long been recognized and it is an intensive research area that has been reviewed extensively [129, 130]. They fall into four drug classes: either quinones, nitroaromatics, aromatic N-oxides or aliphatic N-oxides. The lead agents in each class are at varying stages of clinical development in combination with radiotherapy and standard chemotherapies. These agents are prodrugs that have two key requirements for their biological activation. They require the reductive environment of a hypoxic tumour cell and the appropriate complement of cellular reductase enzymes. Hence they are most commonly called “bioreductive” drugs. The reductase enzymes that have been shown to play a role in bioreductive drug activation include the oxygen-dependent cytochrome P450 family (CYPs), cytochrome P450 reductase (P450R), nitric oxide synthase (NOS), cytochrome b5 reductase and xanthine oxidase. Many bioreductive drugs can also be metabolized by the oxygen-independent enzymes DT-diaphorase (DTD) and nitroreductase. The levels of the majority of these reductase enzymes in tumours are at best variable and often low. Each bioreductive drug also differs in its suitability as a substrate for each enzyme. Therefore, having identified the key reductase enzyme involved, gene therapy can be used to deliver its cDNA, resulting in elevated levels in the tumour and an enhancement of bioreductive drug metabolism. This is termed hypoxia-targeted gene-directed enzyme prodrug therapy (GDEPT) and will target the most treatment resistance tumour fraction, increasing tumour response rates to bioreductive drugs while reducing their potential to cause systemic toxicity.

After years of efforts, tumour hypoxia continues to represent a therapeutic challenge in HNSCC and breast cancer. Nonetheless, the prospect of reducing its impact is looking brighter with the improved ability of detecting and quantifying tumour hypoxia, better understanding of its molecular underpinnings and identification of novel targets for therapeutic exploitation.

In summary, hypoxia results in molecular changes that promote an aggressive phenotype and reduce the efficacy of conventional treatments, resulting in a significant therapeutic challenge.

There remains a need for gene signatures that reflect biological, particularly hypoxia, phenotypes relevant in determining cancer patient prognosis and treatment strategy.

### DISCLOSURE OF THE INVENTION

Using a novel approach that combines knowledge of gene function with analysis of in vivo co-expression patterns, the present inventors have now found a common, compact and highly prognostic hypoxia gene signature of prognostic significance.

Accordingly, in a first aspect the present invention provides a method for assessing a hypoxia phenotype of a tumour of a subject, comprising:
determining the gene expression of between 3 and 50 hypoxia-related genes of a sample obtained from said tumour of the subject, thereby obtaining a sample expression profile of said hypoxia-related genes; and
comparing the sample gene expression profile with a reference expression profile of said hypoxia-related genes, wherein said hypoxia-related genes comprise at least SLC2A1, VEGFA and PGAM1.

As described in detail herein, the hypoxia-related gene signature developed by the present inventors exhibits surprising prognostic power despite its comparatively compact size. For example, the three-gene set SLC2A1, VEGFA and PGAM1 was found to be as prognostic as a much larger gene signature. A compact gene signature that is able to predict tumour hypoxia phenotype and/or prognosis of a subject having a tumour, represents a very significant clinical advance. The compact size permits more efficient, less costly and technically simpler methods of sample analysis, with clear benefits for, e.g. the clinical laboratory setting, personalised medicine and clinical trials of, e.g. hypoxia modifying therapy. Hypoxia gene signatures described previously, such as the 99-gene set of Winter et al., 2007, may not be an optimal solution for assessment of tumour hypoxia phenotype, and patient prognosis. As described further herein, the compact hypoxia gene signature disclosed herein has been found to out-perform previously published signatures in independent datasets of head and neck, breast and lung cancer.

In some cases in accordance with the method of this aspect of the present invention a greater degree of similarity between the sample expression profile and the reference expression profile indicates a greater probability that the tumour of the subject has a hypoxia phenotype.

In some cases in accordance with the method of this aspect of the invention: (i) greater similarity between the sample expression profile and the reference profile (where the reference profile is generated from high grade hypoxia tumours), indicates a greater probability of hypoxia; (ii) higher expression of individual genes or whole signature score vs. reference profile (where the reference profile is generated from e.g. a panel of tumours of varying degrees of hypoxia, and a median cut off level is established) indicates a greater probability of hypoxia.

In some cases according to the method of the first aspect of the invention the hypoxia-related genes comprise, in addition to SLC2A1, VEGFA and PGAM1, at least 2, 3, 4, 5, 10, 15 or at least 20 genes selected from the group consisting of: PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4, FOSL1 and HIG2.

In some cases according to the method of the first aspect of the invention the hypoxia-related genes comprise, in addition to SLC2A1, VEGFA and PGAM1, at least 70%, at least 80%, at least 90%, at least 95% or essentially all of the genes in the group consisting of: PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, KCTD11, ANGPTL4 and FOSL1, which group may or may not include KRT17, PPM1J and/or HIG2.

In some cases according to the method of the first aspect of the invention the hypoxia-related genes consist of the 25-gene set: SLC2A1, VEGFA, PGAM1, PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein PPM1J may optionally be replaced by HIG2.

In some cases the hypoxia-related genes consist of the 26-gene set: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein PPM1J may optionally be replaced by HIG2.

Preferably, the method in accordance with this aspect of the invention employs not more than 50, yet more preferably not more than 40 or 30, and still more preferably, not more than 25 or 26 hypoxia-related genes. The compact hypoxia gene signature may allow the method of the invention to be performed with fewer resources compared with previously-known hypoxia gene signatures.

In some cases in accordance with the method of this aspect of the invention, the method further comprises determining the gene expression of at least 1, 2, 3, 4, 5, or more control genes of said sample. Control genes are typically “house-keeping” genes, e.g. which may be known or suspected to have unchanged expression between hypoxia/normoxia and/or malignant/non-malignant status. Control genes may therefore serve to normalise expression levels of the hypoxia-related genes, e.g. to correct for intra- and inter-assay variation. In some cases, the expression level of the hypoxia-related genes may be a relative expression level determined by dividing the absolute (measured) expression level by the expression level of one or more control genes.

In accordance with the method of this aspect of the invention, the subject is preferably human. The subject may have previously been diagnosed with a tumour, including a solid tumour, which may be cancerous. When the subject is human the genes referred to herein may be taken to refer to the human gene.

In accordance with this and other aspects of the invention, the hypoxia-related genes are designated according their recognised gene symbols (see, e.g., Table 8). The closest Affymetrix probe for each of the hypoxia-related genes is shown in the relevant tables herein (see, e.g. Table 8). For example, the Affymetrix probe for VEGFA is 210512_s_at, for SLC2A1 is 201250_s_at and for PGAM1 is 200886_s_at.

In accordance with this and other aspects of the invention, the hypoxia-related genes may be the human hypoxia-related genes set forth in Table 10 herein. The genes may be selected from any one of the hypoxia-related gene nucleotide sequences as shown in Table 10.

In accordance with this and other aspects of the invention, the control genes may be the human control genes set forth in Table 10 herein. The genes may be selected from any one of the control gene nucleotide sequences as shown in Table 10. Control genes may be referred to herein as “housekeeping genes”, these terms being used interchangeably herein.

In accordance with the method of this aspect of the invention, the tumour of the subject is preferably selected from: a tumour of the head and/or neck, including a head and neck squamous cell carcinoma (HNSCC); a breast tumour; and a lung tumour.

In accordance with the method of this aspect of the invention, the method may comprise the step of obtaining a tissue sample from the tumour of the subject, e.g. by tissue biopsy, or obtaining a liquid sample comprising tumour material (e.g. a blood or interstial fluid sample). In some cases, the method is an in vitro method carried out on a sample of the tumour of the subject which has previously been obtained from the subject. The sample may have been stored (e.g. frozen) and/or processed (e.g. paraffin-embedded) prior to the step of determining gene expression. In some cases, the method comprises, prior to the step of determining gene expression, one or more steps of: extracting RNA (e.g. mRNA) from the sample of the tumour (for example a fresh or processed tissue sample); reverse transcribing RNA extracted from the sample, e.g. to provide cDNA, for subsequent analysis of gene expression by any suitable method.

In accordance with the method of this aspect of the invention, determining the expression of said hypoxia-related genes may comprise quantitative PCR (qPCR). In some cases, the method comprises, prior to carrying out qPCR, extracting RNA from a fresh or processed tissue sample that has been obtained from said tumour and reverse transcribing said RNA. qPCR may, advantageously, be carried out using a set of probes or primers as described herein. Preferably, qPCR may be carried out using a TagMan® qPCR array as described herein. The qPCR may employ a PCR master mix.

In accordance with the method of this aspect of the invention, comparing the sample gene expression profile with the reference expression profile may comprise:
(a) quantitatively comparing the gene expression level of each of said hypoxia-related genes of said tumour with a reference expression level for the respective hypoxia-related gene from a set of tumours of known hypoxia phenotype; and/or
(b) quantitatively scoring the gene expression level of each of said hypoxia-related genes of said tumour, thereby deriving an overall sample score for the sample gene expression profile, and comparing the overall sample score with an overall reference score derived from the expression level of each of said hypoxia-related genes from a set of tumours of known hypoxia phenotype. The expression level of each of said hypoxia-related genes may in some cases be normalised to the expression of one or more control genes. Quantitative comparison of sample and reference gene expression profiles (signatures) may advantageously be carried out using computational methods. In some cases, a probability function and/or a correlation co-efficient may be derived as a measure of similarity. Comparison of similarity with a reference expression profile may involve computing a correlation value (such as a Spearman correlation value) and/or a probability value (such as a posterior class probability value). Typically, a threshold may be set above which a sample expression profile is taken to be classified as sufficiently hypoxic-like and/or which sufficiently meets or exceeds a “hypoxia threshold” that the tumour of the subject is considered to be or have a high probability of being hypoxic. Therefore, in some cases, the method in accordance with this aspect of the invention comprises classifying the tumour of the subject as hypoxic.

In some cases in accordance with the method of this aspect of the invention the method is advantageously combined with one or more conventional methods for assessing tumour hypoxia (e.g. a method as described above under the heading “Current methods for measuring hypoxia”.

In a second aspect, the present invention provides a method for prognosing a subject having a tumour, comprising assessing the hypoxia phenotype of said tumour by a method in accordance with the first aspect of the invention, wherein a greater degree of similarity between the sample expression profile and the reference expression profile indicates a less favourable prognosis for the subject. For example, when the method of the first aspect of the invention indicates that the tumour of the subject is, or is likely to be, hypoxic, this may be taken to indicate that the subject has an aggressive form cancer. Therefore, such a subject may benefit from an aggressive therapeutic, surgical and/or radiologicaly treatment strategy. The method further may comprise recommending and/or carrying out hypoxia-modifying therapy as described above (e.g. any treatment described in the section headed “hypoxia-targeted chemotherapy”).

The method in accordance with the second aspect of the invention may comprise providing a prognosis (e.g. a likely course of disease and/or treatment outcome) based on the degree of similarity between the sample expression profile and the reference expression profile. In some cases, the method comprises determining overall survival time, metastases-free survival time, recurrence-free survival time and/or disease-specific survival time, of the subject.

The method of this and other aspects of the invention may be carried out on a single sample from a single subject, multiple samples from a single subject (e.g. a series of tumour biopsies taken from the same tumour over time or tumour biopsies taken from multiple tumours), a single sample taken from each of a plurality of subjects, or multiple samples taken from each of a plurality of subjects. In particular, the method in accordance with this and other aspects of the invention may comprise assessing the hypoxia phenotype of a tumour from each of a plurality of subjects, and stratifying said plurality of subjects according to the severity of their prognosis. Patient stratification may facilitate prioritising treatments, e.g. to patients categorised as being more likely to benefit from a particular treatment (e.g. hypoxia-targeted chemotherapy). Patient stratification may also be employed in recruitment and/or monitoring of clinical trial subjects for evaluating new therapies (including hypoxia-targeted therapies).

In a third aspect, the present invention provides a method for predicting or assessing response to hypoxia modification therapy in a subject having a tumour, the method comprising assessing the hypoxia phenotype of said tumour by a method in accordance with the first aspect of the invention, wherein a greater degree of similarity between the sample expression profile and the reference expression profile indicates an increased likelihood that the subject will benefit from hypoxia modification therapy.

In a fourth aspect, the present invention provides a set of probes and/or primers for use in a method in accordance with any aspect of the present invention, the set comprising: a plurality of oligonucleotides capable of hybridising to between 3 and 50 hypoxia-related genes, wherein said hypoxia-related genes comprise at least SLC2A1, VEGFA and PGAM1. In some cases in accordance with this aspect of the invention, the set comprises or consists of primers or probes that hybridise (e.g. hybidise under stringent conditions) and/or which comprise an oligonucleotide sequence of 10 to 50 (preferably 15 to 30) contiguous nucleotides of a nucleotide sequence having at least 90%, at least 95%, at least 99% or 100% identity to the sequence of any one of the hypoxia-related genes identified herein, particularly any one of the 26-gene set of hypoxia-related genes consisting of: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein PPM1J may optionally be replaced by HIG2. Preferably, said sequence identity is calculated over the full-length of the oligonucleotide probe. Preferably, the set in accordance with this aspect of the invention may comprise the closest Affymetrix probe for each of the hypoxia-related genes as shown in the tables herein. For example, the set in accordance with this aspect of the invention may comprise the probes identified by the following Affymetrix designations: 210512_s_at (for VEGFA), 201250_s_at (for SLC2A1) and 200886_s_at (for PGAM1). Preferably, the set in accordance with this aspect of the invention consists of a set of oligonucleotides that, in total, recognise not more than 50 (preferably not more than 40, not more than 30, and yet more preferably not more than 25 or 26) hypoxia-related genes as defined herein, particularly the 26-gene set of hypoxia-related genes consisting of: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein said PPM1J may optionally be replaced by HIG2.

In some cases in accordance with this aspect of the invention, the set comprises or consists of, in addition to primers and/or probes directed to SLC2A1, VEGFA and PGAM1, primers or probes that hybridise (e.g. hybidise under stringent conditions) and/or which comprise an oligonucleotide sequence of 10 to 50 (preferably 15 to 30) contiguous nucleotides of a nucleotide sequence having at least 90%, at least 95%, at least 99% or 100% identity to the sequence of at least 2, 3, 4, 5, 10, 15 or at least 20 genes selected from the group consisting of: PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein said PPM1J may optionally be replaced by HIG2.

In some cases in accordance with this aspect of the invention, the set comprises or consists of, in addition to addition to primers and/or probes directed to SLC2A1, VEGFA and PGAM1, primers and/or probes directed at least 70%, at least 80%, at least 90%, at least 95% or essentially all of the genes in the group consisting of: PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, KCTD11, ANGPTL4 and FOSL1, which group may or may not include KRT17, PPM1J and/or HIG2.

Preferably, the set in accordance with this aspect of the invention comprises or consists of primers and/or probes directed to the set of hypoxia-related genes that consists of: SLC2A1, VEGFA, PGAM1, PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein said PPM1J may optionally be replaced by HIG2.

Preferably, the set in accordance with this aspect of the invention comprises or consists of primers and/or probes directed to the set of hypoxia-related genes that consists of: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1.

In some cases in accordance with this aspect of the invention, the set further comprises probes and/or primers capable of hybridising to 1, 2, 3, 4, 5, or more control genes. The control genes may be selected from “house-keeping genes” that are not, or thought not to, have altered gene expression as a result of hypoxia and/or cancer-related phenotype changes.

In some cases in accordance with this aspect of the invention, the set of probes and/or primers may be provided in an array on a solid support or may be coupled to a plurality of labelled beads.

In accordance with this and other aspects of the invention, the hypoxia-related genes may be the human hypoxia-related genes set forth in Table 10 herein. The genes may be selected from any one of the hypoxia-related gene nucleotide sequences as shown in Table 10.

In accordance with this and other aspects of the invention, the control genes may be the human control genes set forth in Table 10 herein. The genes may be selected from any one of the control gene nucleotide sequences as shown in Table 10.

In a fifth aspect, the present invention provides a TaqMan® qPCR array for use in a method according to any aspect of the present invention, the array comprising a micro-fluidic card pre-loaded with primers for amplification of:
between 3 and 50 hypoxia-related genes, wherein said hypoxia-related genes comprise at least SLC2A1, VEGFA and PGAM1; and
   optionally, one or more control genes that are not hypoxia-related. In some cases, the micro-fluidic card may be pre-loaded with primers for amplification of:
the 26-gene hypoxia signature set consisting of: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1; and
optionally, one or more control genes that are not hypoxia-related.

In some cases in accordance with this aspect of the invention, said micro-fluidic card is pre-loaded with primers for amplification of, in addition to SLC2A1, VEGFA and PGAM1, at least 70%, at least 80%, at least 90%, at least 95% or essentially all of the genes in the group consisting of: PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, KCTD11, ANGPTL4 and FOSL1, which group may or may not include KRT17, PPM1J and/or HIG2; and
optionally, one or more control genes that are not hypoxia-related.

In some cases in accordance with this aspect of the invention, said micro-fluidic card is pre-loaded with primers for amplification of:
the 25-gene hypoxia signature set consisting of: SLC2A1, VEGFA, PGAM1, PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein PPM1J may optionally be replaced by HIG2; and
optionally, one or more control genes that are not hypoxia-related.

In accordance with this and other aspects of the invention, the hypoxia-related genes may be the human hypoxia-related genes set forth in Table 10 herein. The genes may be selected from any one of the hypoxia-related gene nucleotide sequences as shown in Table 10.

In accordance with this and other aspects of the invention, the control genes may be the human control genes set forth in Table 10 herein. The genes may be selected from any one of the control gene nucleotide sequences as shown in Table 10.

In a sixth aspect the present invention provides a kit for use in a method in accordance with any aspect of the present invention, the kit comprising:
a set in accordance with the fourth aspect of the invention or the TaqMan® qPCR array in accordance with the fifth aspect of the invention; and
instructions, controls and/or reagents for performing a method according to any aspect of the invention.

These and further aspects and embodiments of the invention are described in further detail below and with reference to the accompanying examples and figures.

### DESCRIPTION OF THE FIGURES

FIG. 1 shows Hypoxia gene-expression network in HNSCC (Vice 125 data set). Seeds (yellow) and learnt genes (blue) are shown; circle size is proportional to C score. Solid edges connect cluster members with seeds; length is proportional to membership, colour represents Spearman correlation (blue, −1; red, +1). Green dotted edges connect seeds; their length is proportional to the shared neighbourhood.

FIG. 2 shows the hypoxia network mapped onto Reactome pathways (A) coloured by increasing C score from dark blue to bright red; and validation of up-regulated HNSCC (B) and BC (C) signatures by comparison with the literature. The proportion of literature-validated genes is shown as function of the number of top-ranked (by C score) genes considered; standard errors estimated by bootstrap.

FIG. 3 shows common hypoxia signature of 51 genes. (A) Hypoxia/normoxia expression ratio in endothelial, smooth muscle, human mammalian epithelial, renal proximal tubule epithelial cells (EC, SMC, HMEC, RPTEC); and in (B) HIF1a/HIF2a siRNA experiment. (C, D) Connectivity-ranked forest plots: metastases- and recurrence-free survival (MFS, RFS) hazard ratio (HR) (red) with 95% confidence intervals, and HRs if permuted list (black). Control: random sampling of N=51 genes (original magnification, x100).

FIG. S1 shows validation of in-vivo hypoxia signature (HS) using Reactome pathway database. A) The complete chart of the Reactome pathway database (www.reactome.org) is shown with mapping of genes with top-ranked connectivity, C, score in HN Vice125 dataset (Table 1). The names of pathways represented in the signature are shown. Colouring is done according to the average values of all identifiers linked to that reaction. A) Colouring from dark blue to bright red indicates increasing C rank. B) Colouring indicates direction of regulation: consistently up-regulated reactions are in red, consistently down-regulated in blue, green represent reactions where some up-regulated and some down-regulated genes were observed.

FIG. S2 shows the overlap between pairs of seed clusters (ie. the S score) is plotted as a function of the correlation between the expression values for the same pair of seeds. The seeds were set to the ‘literature list’ http://cancerres.aacrjournals.org/cgi/data/67/7/3441/DC1/1); Vice125 dataset was used (Table 1).

FIG. S3 shows comparison of the results from the literature validation of the hypoxia signatures obtained using a range of different methods for clustering, multiple test correction, and initial seed choice. The “literature list” was our literature reference (5). The Vice125 dataset was used (Table 1). Data were pre-processed using GCRMA (A) or MAS5 (B). SL_{—}1 and 2 are respectively set B and A described in Table S1. The attribute “median” indicates that when more than one probeset mapped to the same gene, the “median” criterion was used to assign the expression to the initial seed for that gene rather than the default “best candidate” criterion (see Suppl. Methods section). Pearson or Spearman correlation were used as clustering distance metrics, with either Bonferroni correction for multiple testing or false discovery rate correction permutation of the samples. In all cases data were filtered for unspecific probesets and low expression probesets as indicated in the Suppl. Methods.

FIG. S4 shows frequency distributions for the connectivity score C of the hypoxia networks trained in head and neck and breast cancer datasets (Table1). The distribution of the mean values of C after bootstrapping (n=300) is shown for genes on the array that passed initial filtering (see Suppl. Methods). Seed choice A in Table S1.

Comments to FIG. S4: properties of connectivity C score The distribution of C for all genes was found to be highly skewed towards zero in all datasets considered irrespectively of seed choice, filtering, bootstrapping, pre-preprocessing or clustering methods (data not shown). Thus, as expected, most genes represented on the array do not cluster with any of the seeds, and the probability of a gene being a member of one or more of the seed clusters is extremely small. Both skewness and maximum value of the distribution of C varied between datasets; this is due to various factors including the difference in size of the datasets, the difference in population, the difference in size and the size and generation of Affymetrix arrays considered. For example, C was less skewed in GSE65320xf and GSE6532KI. These are between two and three times larger than the other datasets (Table 1). It is possible that some true correlations are not found to be significant in the smaller datasets. Furthermore, these two datasets use smaller arrays (Table 1) containing a subgroup of relatively well-characterised transcripts; thus the proportion of transcripts in these arrays which are involved in cancer metabolism-related pathways, and which cluster at least with one of the seeds, might be higher. However, the maximum C score is similar between these and the other datasets suggest that only genes with a lower C score, that is the potential false positives, are missed out, but not the ones with a high C score which are the ones we believe to be the real positive for hypoxia in-vivo. To confirm this, a pair-wise comparison between HG U133a and HG U133-plus2 training datasets (excluding GSE6791 where samples are processed using a different protocol, as discussed in the next sections) of the top-ranked genes showed that the overall overlap between datasets is higher when top C scores were considered (median overlap for genes with C>0.4 is 12%) than when lower scores are included (median overlap for genes with C>0.2 is 3%). Different is the case of dataset GSE2379, where a much lower C score maximum is observed. This dataset uses Affymetrix arrays of older generation, and it is much smaller than the other datasets (Table 1), approaching the minimum size needed to apply the present method (when using 20 samples the minimum correlation which can be detected at 0.05 significance level and with a 90% power is r=0.66).

FIG. S5. Prognostic significance of hypoxia meta-signatures (HMS) from head and neck and breast datasets. Cumulative forest plots of Hazard Ratio (HR) and 95% confidence limits of the MHS score in a Cox multivariate analysis including other clinical prognostic factors are shown for the HNSCC HMS (A and C) and the breast cancer HMS (B and D). HR are shown in red, the back dots are the HRs for the permuted list. For details on the methods used to build these plots see text and FIG. 4. Results are shown for the NKI and GSE2034 datasets (Table 1); metastases-free survival, MFS, and recurrence-free survival, RFS, are considered respectively. The control shown at the bottom of the plots is the average HR when randomly resampling (n=100) a number of genes equal to the full signature. Seed choice was A in Table S1.

Note: Colour references herein are for reference only; the figures do not use colour.

### DETAILED DESCRIPTION OF THE INVENTION

The following is presented by way of example and is not to be construed as a limitation to the scope of the claims.

### EXAMPLES

### Example 1

### Deriving a Hypoxia Gene Expression Signature

### Large-Meta Analysis of Multiple Cancers Reveals a Common, Compact and Highly Prognostic Hypoxia Metagene

Introduction

Gene-expression studies attempt to extrapolate biologically and clinically relevant hypotheses from gene expression patterns. However, many current studies make little use of existing knowledge such as gene function within specific pathways, and prognostic signatures are often derived with no reference to the functional roles of their components.

One increasingly popular method that aims to make use of prior knowledge is Gene Set Enrichment Analysis (GSEA) (Subramanian et al, 2005). GSEA first conducts a supervised analysis by ranking genes according to their ability to discriminate between different sample groups, and then maps them onto previously defined gene-sets, typically formed according to common function using annotation sources. The goal is to identify sets containing a statistically significant number of highly ranked genes, and then to use this information to provide functional characterizations for the samples in question. Although powerful, GSEA relies on stratification of the experimental samples into distinct groups, often making it unsuitable for use with heterogeneous clinical datasets.

Another approach often applied to microarray data involves creation of a co-expression network within which each ‘node’ represents a gene, and ‘edges’ are created between genes when their expression patterns are significantly correlated. Co-expression networks have been used to formulate functional and clinical hypotheses from in vivo data (Butte & Kohane, 2003; Hahn & Kern, 2005; Wolfe et al, 2005). A disadvantage with the approach is that it can be susceptible to the multiple testing issues that arise due to the large number of genes represented on a typical microarray. Setting a low threshold for a significant correlation between genes will result in the inclusion of many spurious links, while a high threshold will control the false positive rate at the expense of omitting many genuine edges.

Here we illustrate and validate a network-based approach with parallels to both GSEA and co-expression networks; for a workflow of the method see Suppl. Material and Methods. It can be applied directly to clinical data, even when the samples cannot be partitioned in advance into distinct groups. The algorithm begins with a collection of ‘seed’ genes that are then used as starting point from which to build an association network. Rather than simply connect gene pairs with high correlation between their expression profiles, the approach defines a “neighborhood of co-expression” around each seed gene, and then connects seeds that have a significant degree of overlap between their neighborhoods. This approach is relatively robust against the inclusion of spurious edges, since edges are only added when there is consistently high correlation to many intermediate genes that form the intersection between seeds. We previously used a seed-based approach successfully to predict hypoxia-related genes (Winter et al, 2007); the current study develops the method in a meta-analysis context to produce robust signatures requiring fewer genes, making them more suitable for clinical use, for example in quantitative RT-PCR analyses of biopsies at presentation.

Hypoxia plays a key role in defining the behavior of many cancers including Head and Neck Squamous Cell Carcinomas (HNSCC) (Nordsmark et al, 2005) and breast carcinomas (BC) (Fox et al, 2007); thus the identification of common hypoxia-regulated genes is important both for understanding of cancer evolution, and for improved prognosis or development of novel therapies. The described approach was applied to a large meta-analysis of HNSCCs and BCs to successfully define a common and robust hypoxia signature.

Materials and Methods

Seed Clustering

The process begins with k seed genes, Π={π₁, π₂ . . . π_{K}} (‘gene’ is used throughout for convenience, although ‘transcript’ is generally more accurate). Spearman correlation, ρ, is computed between seeds and genes Y={y₁, y₂ . . . yₘ} in a dataset of n samples, X={x₁, x₂ . . . xₙ}. For each seed/gene pair, their ‘affinity’ is defined as:

$$δ ( {π}_{i} , {y}_{i} ) = {[ 1 + {e}^{\frac{( {ϑ}_{t} - {{ρ}_{{π}_{i} , {y}_{j}}}^{2} )}{{ϑ}_{s}}} ]}^{- 1} \qquad ( Equation 1 )$$

where θₜ and θₛ define extent and sharpness of the cluster. When θₛ→0, δ reduces to the step function with δ=0 if ρ²<θₜ, δ=1 if ρ²>θₜ. In this limit, the method is parameter-free, and this will be used in this study. θₜ is defined objectively using a probability threshold, α, of observing a given correlation if the null hypothesis (i.e. no association) was true. This needs to be corrected for multiple testing (Hastie et al, 2001) to account for the size of Y; here, α=0.05 after Bonferroni correction was considered. Finally, a membership function is defined:

γ(yᵢ,πₖ)=δ(yᵢ,πₖ)/Σⱼ₌₁^{K}δ(yᵢ,πⱼ) (Equation 2)

An increasing γ indicates stronger membership of a gene to a seed cluster.

Shared Neighborhood

The shared neighborhood, S, between two seeds is defined as:

$$S ( {π}_{i} , {π}_{j} ) = \frac{{\sum }_{k = 1 ; k \neq i , j}^{m} min [ γ ( {π}_{i} , {y}_{k} ) , γ ( {π}_{j} , {y}_{k} ) ]}{{\sum }_{k = 1 ; k \neq i , j}^{m} max [ γ ( {π}_{i} , {y}_{k} ) , γ ( {π}_{j} , {y}_{k} ) ]} \qquad ( Equation 3 )$$

where γ is the membership (Eq. 2). Two seeds are considered to carry a high degree of related information if their clusters share many genes (high S values). A sign function is also defined:

$$F ( {π}_{i} , {π}_{j} ) = \frac{\begin{matrix}{\sum }_{k = 1 ; k \neq i , j}^{m} min [ γ ( {π}_{i} , {y}_{k} ) , γ ( {π}_{j} , {y}_{k} ) ] \cdot \\ sgn [ ρ ( {π}_{i} , {y}_{k} ) \cdot ρ ( {π}_{j} , {y}_{k} ) ]\end{matrix}}{{\sum }_{k = 1 ; k \neq i , j}^{m} min [ γ ( {π}_{i} , {y}_{k} ) , γ ( {π}_{j} , {y}_{k} ) ]} \qquad ( Equation 4 )$$

where sgn(x) is the sign function:—sgn(x)=1 if x>0, sgn(x)=−1 if x<0. If two seeds are correlated with their shared features in the same direction, F=1 (seeds are fully concordant); if they are correlated with their shared features in opposite direction, F=−1.

Seed-Dependent Connectivity

The strength of the relationship between a gene and the whole set of seeds is estimated using the connectivity function:

$$C ( {y}_{i} ) = \frac{{\sum }_{j = 1 ; j \neq i}^{K} w ( {π}_{j} ) \cdot γ ( {y}_{i} , {π}_{j} )}{{\sum }_{h = 1 ; h \neq i}^{K} w ( {π}_{h} )} \qquad ( Equation 5 )$$

where γ is defined in Eq. 2 and w are weights which regulate the importance of each seed. In this study, we consider w=1, unless yᵢ is one of the seeds, or a probeset biding to the same transcript as the seed; in this case, to avoid bias, for that seed w=0.

A connectivity score, is defined as the fractional rank of C; that is the ranking normalized between 0 (lowest C) and 1 (highest C).

Bootstrapping, Monte-Carlo and Meta-Connectivity Score

Random sets of seeds are generated by Monte-Carlo sampling, clusters aggregated around them, C and S calculated. This procedure is repeated to generate null distributions and it provides an estimate of the probability of observing by chance a given value of C and S.

Bootstrapping is re-sampling with replacement of the original population; it is used to provide maximum likelihood best estimates when an analytical approach is not feasible (Hastie et al, 2001). Here, it is used to provide best estimates and confidence limits for C and S. These are used in a meta-analysis across several datasets to define a meta-connectivity score as:

$$\hat{C} ( {y}_{i} ) = \frac{{\sum }_{h = 1}^{Nd} {R [ C ( {y}_{i} ) ]}_{h} / {σ}_{h}^{2}}{{\sum }_{h = 1}^{Nd} 1 / {σ}_{h}^{2}} \qquad ( Equation 6 )$$

where R[C(yᵢ)]ₖ is the fractional rank of C (Eq. 5), N_{d} is the number of datasets, σ²ₖ is the variance of the ranked C, R[C(yᵢ)]ₖ, in dataset k for gene yᵢ.

A common metagene between tumours types is derived by taking the Ĉ scores product, [symbol] Ĉ. This is effectively a rank product, as Ĉ is an average rank (Eq. 6). A common metagene between tumours types is derived by taking the Ĉ scores product, [symbol] Ĉ. This is effectively a rank product, as Ĉ is an average rank (Eq. 6).

Cumulative Forest Plots Based on Connectivity Score

A summary expression score, E, is defined in each sample as the median of the absolute expression of the genes in the signature. The median is used as summary statistics to reduce the effect of outliers. A cumulative forest plot is defined:—genes are added to the signature, one by one, in order of their connectivity, C, score so that genes that are introduced first have the highest connectivity. At each step, a summary expression, E, is derived using the new gene and genes from the previous steps. Samples are then ranked by their E value; this assigns a hypoxia score (HS) from lowest (least hypoxic) to highest (most hypoxic). HS is then renormalized between 0 and 1; introduced into a Cox multivariate analysis that includes the other significant clinical covariates; and the hazard ratio (HR) of the HS is calculated.

Datasets, Data Processing and Annotation

NCBI Gene Expression Omnibus (http://www.ncbi.nlm.nih.gov/geo/) was searched for gene expression studies in cancer, published in peer-reviewed journals, where microarray were performed on frozen material extracted before chemotherapy, radiotherapy or adjuvant treatment. Eight datasets (Table 1) were selected that used similar platforms (Affymetrix U133A, B and plus2). Processing was performed using simpleaffy (Wilson & Miller, 2005); the gcrma function was used to estimate expression values, data were quantile-normalized and logged (base2). Other datasets were identified for validation in which different technologies were used (Table 1); non-Affymetrix datasets were processed as described in the original publications. More details on pre-processing and annotation are given in the supplementary methods.

Results

Derivation of a Hypoxia Expression Network

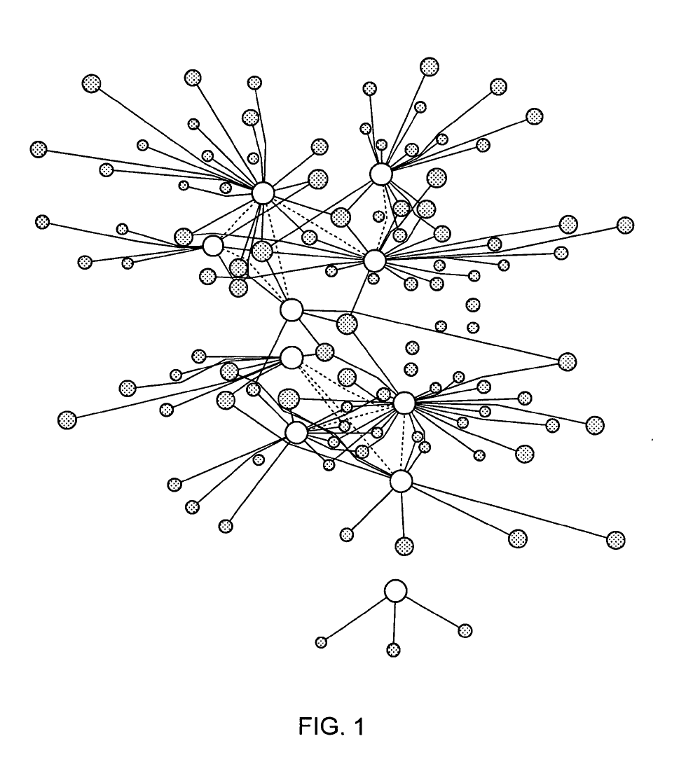

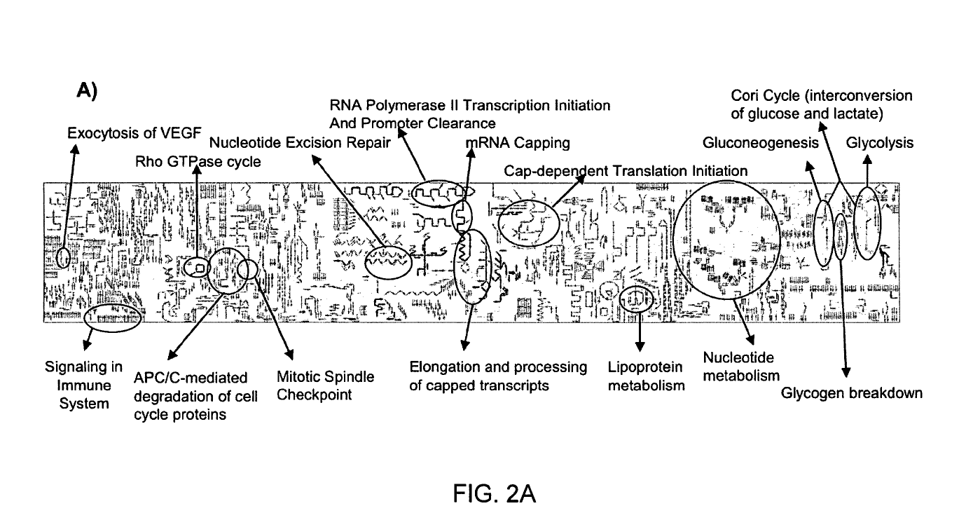

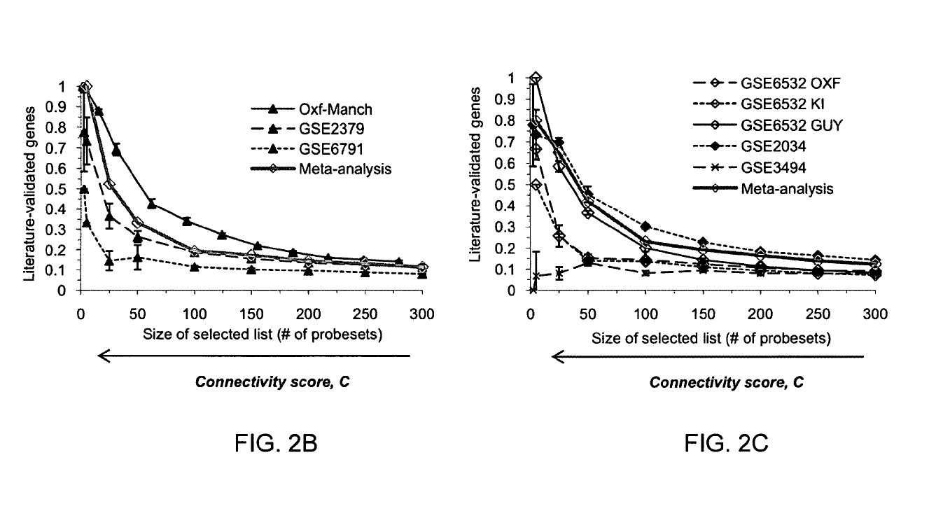

A hypoxia expression network was built first in a dataset comprising 59 HNSCC tumour samples (Vice 125; Table 1) using well-characterized hypoxia-related genes identified from the literature covering a comprehensive set of hypoxia-induced pathways (set A, Table S1). These were adrenomedullin (ADM), adenylate kinase 3-like 1 (AK3L1), BCL2/adenovirus E1B 19 kDa interacting protein 3 (BNIP3), carbonic anhydrase IX (CA9), enolase 1 (ENO1), hexokinase 2 (HK2), lactate dehydrogenase A (LDHA), phosphoglycerate kinase 1 (PGK1), solute carrier family 2 member1 (SLC2A1), and solute carrier family 2 (VEGFA). The resultant network (FIG. 1) was observed to map to distinct regions of the Reactome (www.reactome.org) network and to several hypoxia-related pathways (FIGS. 2 and S1). The method was applied to additional HNSCC and BC training datasets (Table 1) with similar results (Table S2).

In the resulting expression networks, high shared neighborhood, S (Equation 3), values between seed-pairs were generally associated with a high pair-wise correlation. However, this relationship did not always hold. An example is given in FIG. S2, where genes in a published 245-gene literature list (LL) (Winter et al, 2007), were used as starting seeds. Many of the seeds with high pair-wise S but low correlation appeared in the same KEGG (http://www.genome.jp/kegg/) pathway but would not be detected in a straightforward correlation analysis (FIG. S2). Furthermore some seeds showed markedly different in vivo and in vitro behaviors; for example, PFKFB3 (set B, Table S1) did not have significant overlap with any other seeds, while CCNG2 showed a consistent inverse-correlation with other seeds (F<0; Equation 4) supporting results from previous studies (Choi & Chen, 2005). Thus, the method was able to identify seeds that behave differently from their peers; for the rest of this study, only the conservative seed set A was used. This set showed higher pair-wise S values than any other set of randomly selected seeds (repeated 1000 times) from the 245-gene LL.

Seed-Dependent Connectivity Identifies a Hypoxia Signature

Genes in the co-expression networks were ranked by their connectivity score, C (Equation 5), and compared with the hypoxia 245-gene LL. As the latter is biased towards up-regulated genes (Harris, 2002), only genes showing consistent positive correlation with the initial seeds were considered. To avoid bias, the initial seeds were excluded from this comparison. The relative proportion of known hypoxia genes increased with increasing connectivity, C, score (FIG. 2), confirming its utility as a metric for predicting functional relationships. Similar results were observed with different clustering and pre-processing methods (FIG. S3). However, differences were observed between datasets. Much of this inter-experimental variation is likely to reflect differences in both the patient populations and the processing of the biological material. For example, both datasets GSE6791 and GSE3494, which showed a lower level of enrichment for hypoxia genes than others, featured samples with the highest proportions of tumour cells selected either by micro-dissection or visual scoring.

Next we selected a subset of ‘hub’ genes from the hypoxia network, with the goal of using them as a hypoxia signature. Genes with high connectivity, C (Equation 5), score (p<0.01, estimated by Monte-Carlo simulation) were considered (Table S2). Each of these genes had a greater-than-expected overlap with the neighborhoods of all other genes in the network (FIG. S4). The seeds were only selected if they were hubs with respect to all other seeds. Using the Reactome database we confirmed that pathways known to be regulated by hypoxia, such as glycolysis, gluconeogenesis, glucose metabolism and Cori Cycle (recycling of lactic acid) were consistently over-represented in these genes (FIG. 2 and Table S3). Similarly, GO analysis (http://genecodis.dacya.ucm.es) found over-representation (false discovery rate <0.05) of pathways such as glycolysis, phosphoinositide-mediated signaling, nuclear mRNA splicing, translational initiation, regulation of cell cycle, ubiquitin-dependent protein catabolism, apoptosis and regulation of cell proliferation. Over-represented molecular functions included ATP binding, nucleotide binding, lipoic acid binding, oxidoreductase and L-lactate dehydrogenase activity.

Meta-Signature Enrichment and the Prognostic Value of Compact Signatures

We selected genes that showed consistent high connectivity across datasets and derived meta-signatures for hypoxia in HNSCC and BC. Interestingly, although some of the datasets performed poorly on their own, meta-analysis signatures were robust to their inclusion and performed well (FIGS. 2B, C).

We assessed the meta-signatures' prognostic relevance in four independent datasets (Table 1). Samples were ranked using a summary expression score, E, of the genes in the signature; this produced a hypoxia score, HS, which assigns a hypoxic status to the tumours in the validation datasets. Multivariate Cox analysis including available clinical factors was carried out using each dataset; clinical variables were selected using backward-stepwise maximum likelihood. The HS was introduced into the reduced clinical model to estimate the prognostic significance of the meta-signatures independently from other clinical variables (FIG. S5 and Table S4).

To address whether smaller signatures with equal prognostic ability could be derived by using a more stringent C-score, cumulative forest plots were generated in which genes were introduced into the HS calculation one-by-one, in decreasing order of their meta-C score (FIG. S5). Only a few genes were needed before the hazard ratio stabilized and a reduced signature was found to be at least as prognostic as a larger one (FIG. S5). Interestingly, when genes were introduced into the cumulative plots in random order, rather than by their ranked C-score, more genes were needed to reach equivalent prognostic significance (FIG. S5).

A Common Hypoxia Metagene Across Cancer Types

Common hubs in HNSCC and BC were selected by considering, for each gene, the product, [symbol] C, of the C-scores between the HNSCC and BC meta-analyses. A common metagene was derived by considering genes with [symbol] C>0.5 (Table 2 and S5). This hard cut-off was chosen since a gene with a [symbol] C score approaching that which would be expected by chance ( [symbol] C≈0.5) in one tumour site, would have to achieve a maximal score in the other tumour site to be included.

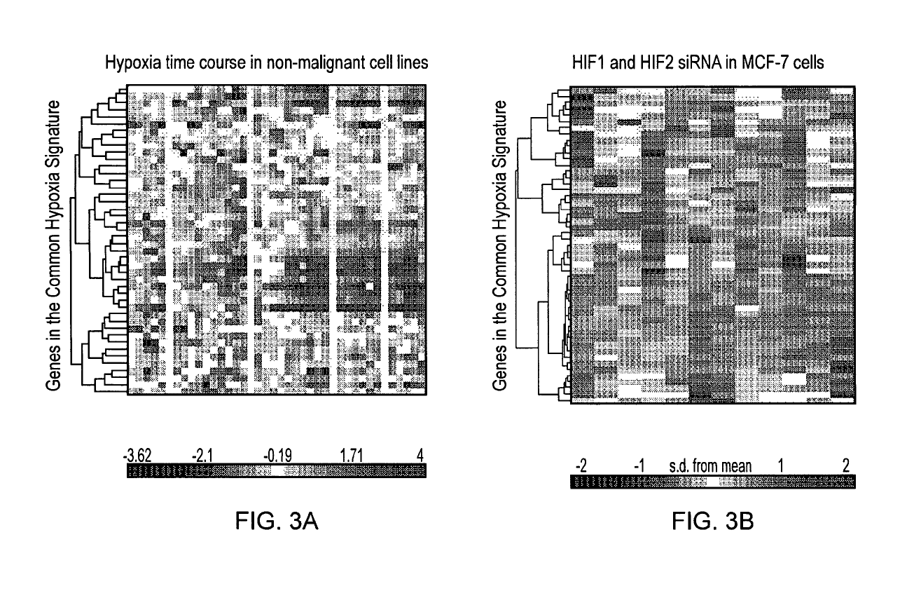

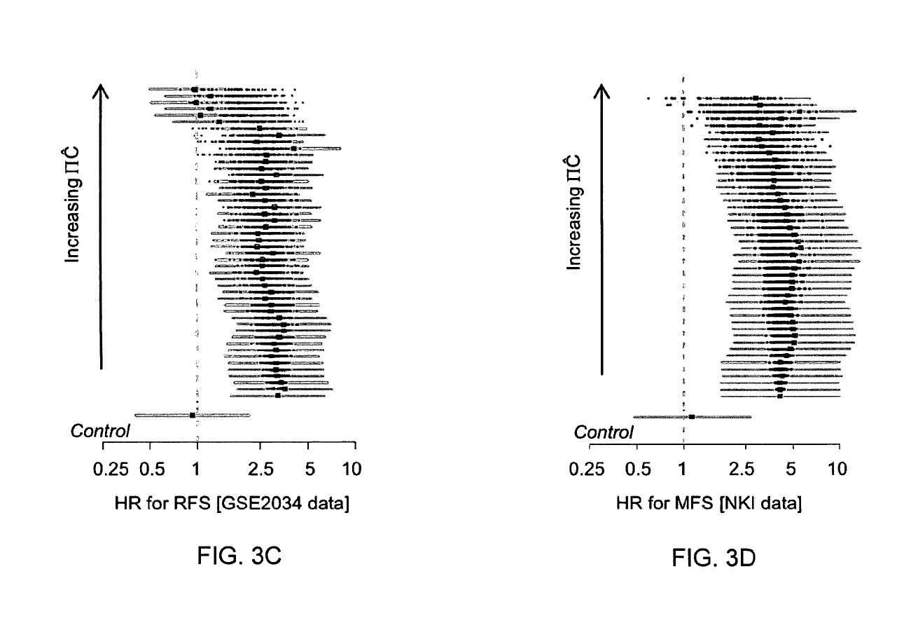

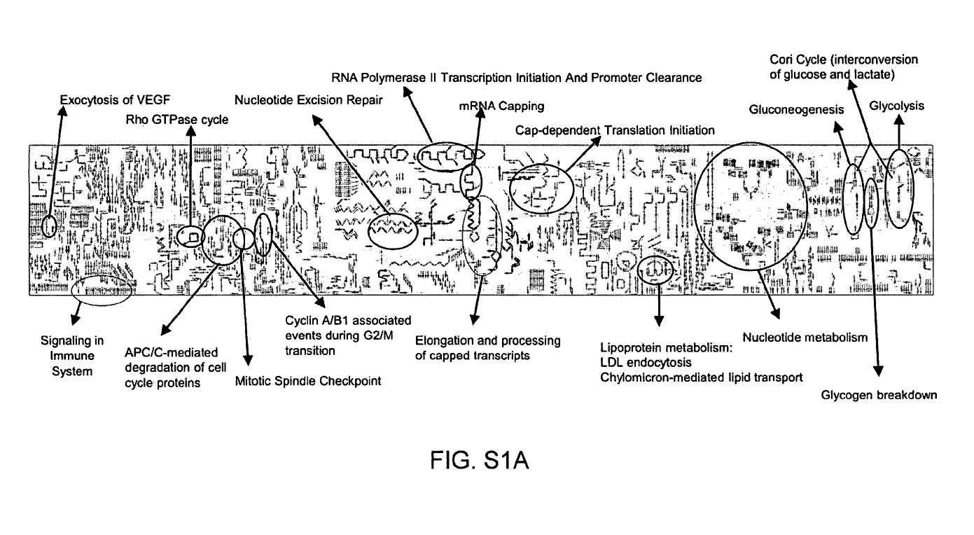

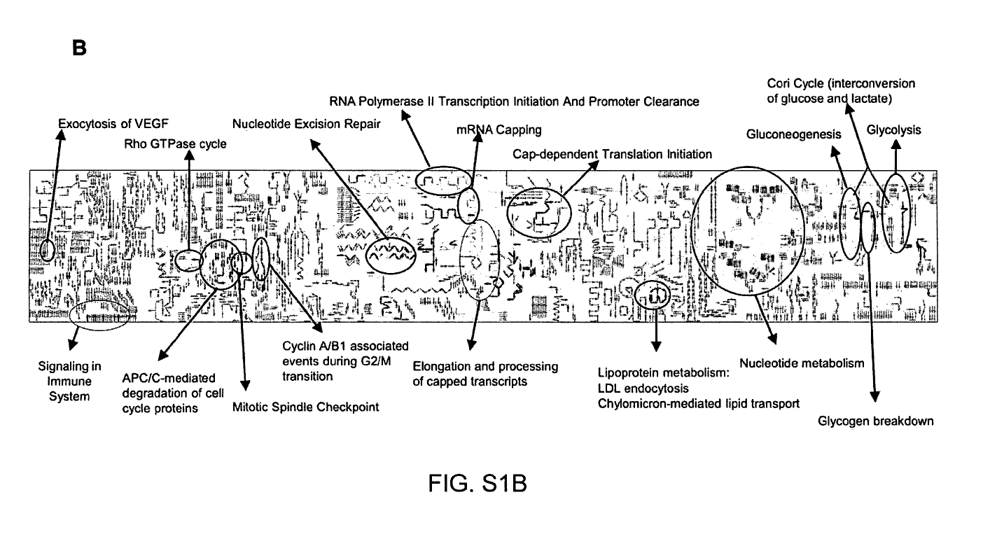

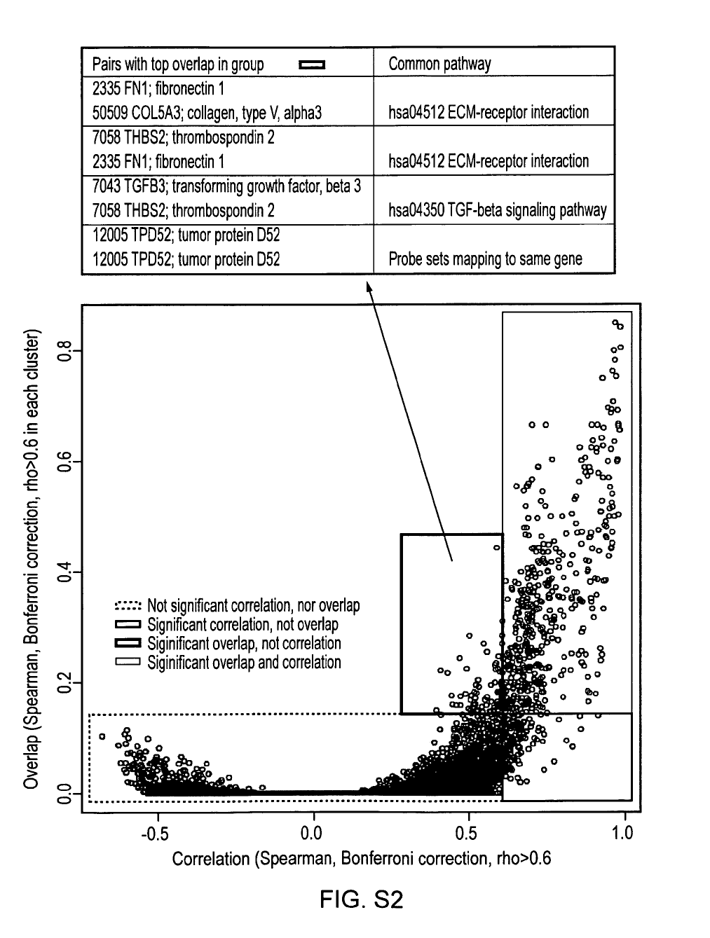

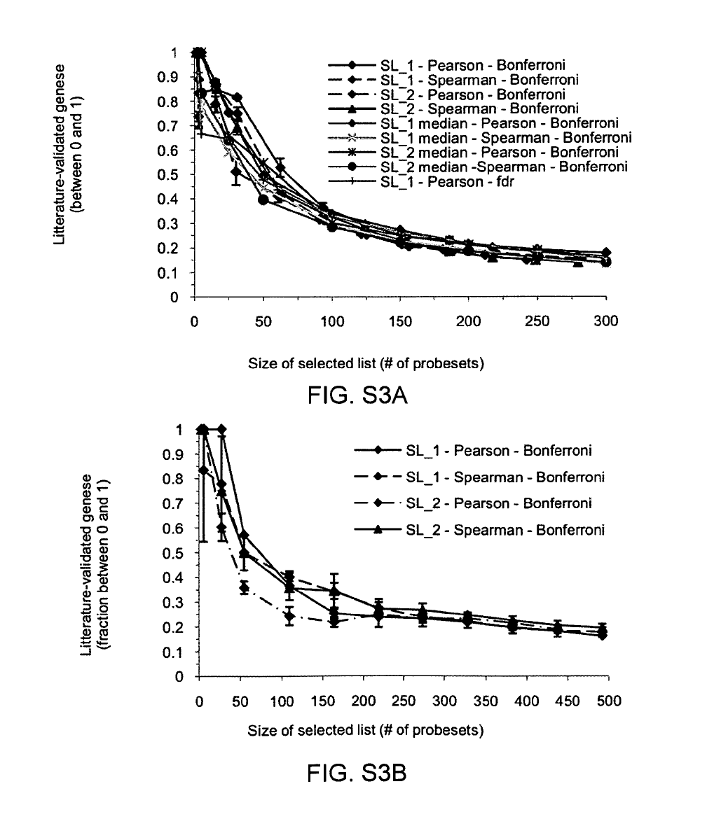

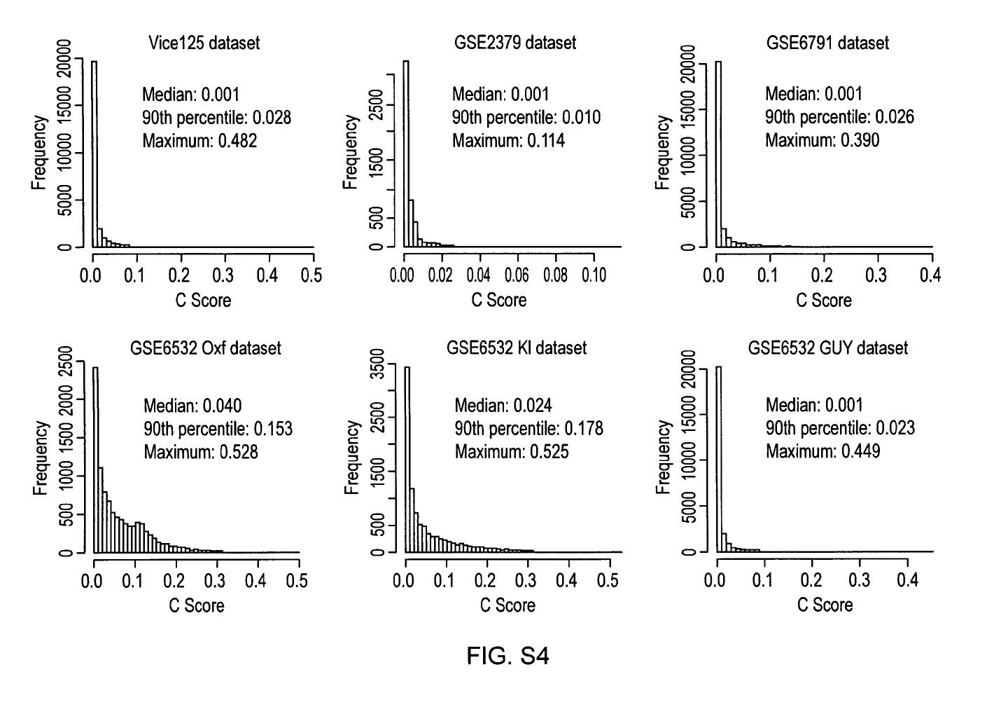

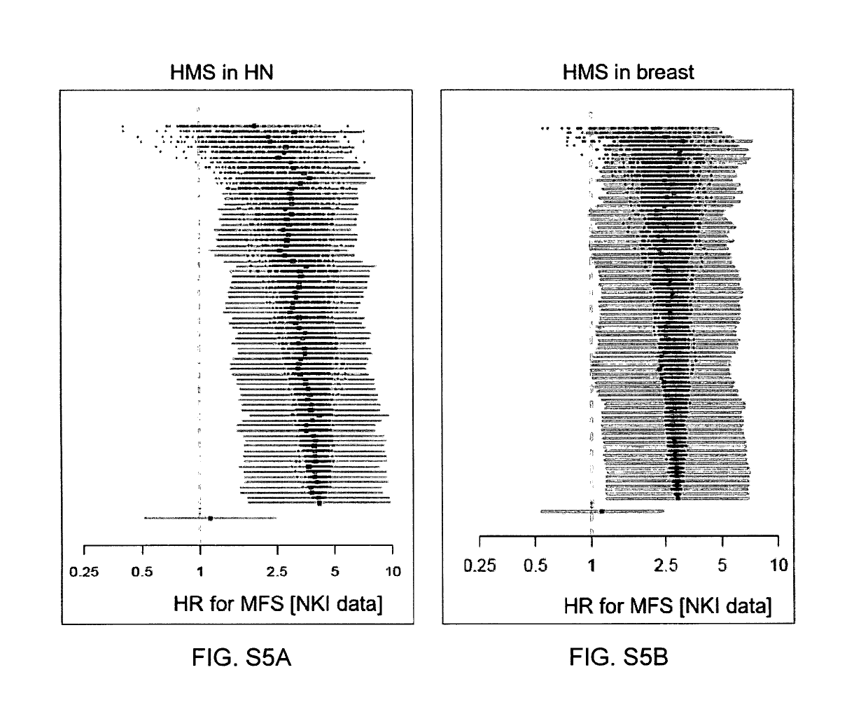

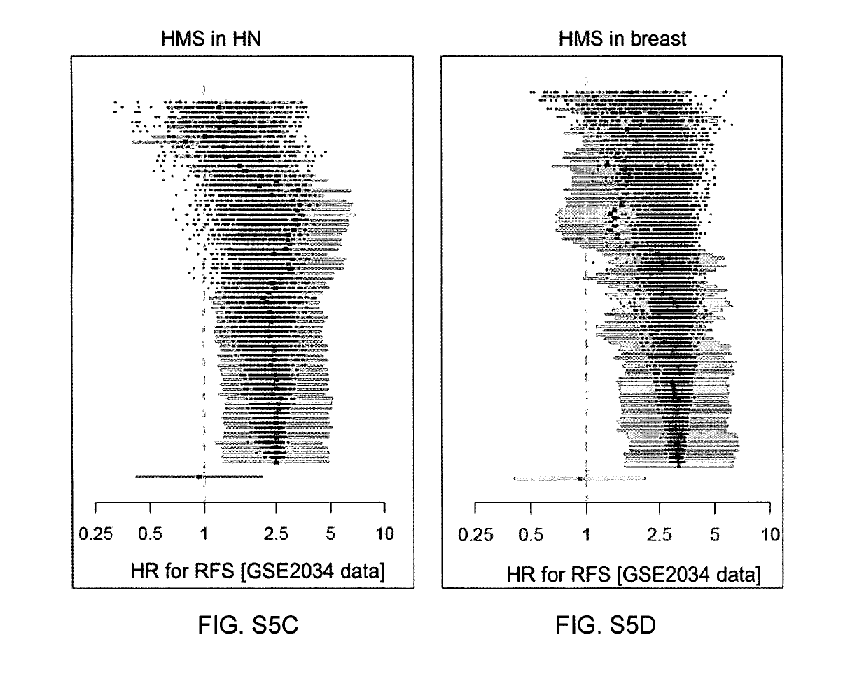

We investigated in cell lines potential regulation of genes in the common metagene by hypoxia and by HIF1a, the main mediator of the hypoxia response in cancer. We considered two datasets: a hypoxia time course in a panel of epithelial and endothelial non-malignant cells (Chi et al, 2006), and a HIF1a and HIF2a siRNA experiment in MCF7 BC cells (Elvidge et al, 2006) exposed to hypoxia. For details of these data we refer to the original publications. Although differences between cell lines and BC in vivo are expected, a high proportion of genes in the common metagene (38/51) showed either regulation in the hypoxia time course or in the siRNA experiment (FIGS. 3A, B and Table S5). Several of these genes were also predicted as HIF1a targets and showed potential HIF1a binding sites (Table S5). Furthermore, 22 had already been found hypoxia-regulated by previous published work (Table S5). Overall approximately 80% (42/51) of genes in the common metagene were confirmed by at least one validation, several of them by more than one.

The common hypoxia metagene (51 genes) was prognostic in independent datasets of different cancer types (Table 3) and showed greater prognostic power than (i) an in-vitro derived hypoxia signature (Chi et al, 2006); (ii) the initial seeds and (iii) our 99-gene HNSCC hypoxia metagene derived previously (Winter et al, 2007) (Table 3). A signature derived by selecting genes co-expressed with VEGF in BC (Desmedt et al, 2008) had no independent prognostic significance (data not shown), in agreement with the published study. In a further validation using Oncomine (http://www.oncomine.org), all but one of the fifteen top-ranked (by HC score) genes showed prognostic significance in at least one tumour site (p<0.0001). The only top gene for which prognostic significance was not reported in Oncomine, SLC2A1 (GLUT1), is prognostic in other studies (Oliver et al, 2004).

Finally, cumulative forest plots based on connectivity score (FIG. 3) showed no further improvement in hazard ratio after addition of a small number of genes. Although differences were observed between HNSCC, BC and lung cancers, we found in all cases that a common signature reduced to a small number of [symbol] C score top-ranked genes was at least as prognostic as the full signature (FIGS. 3C, D and Table 3).

Discussion

Hypoxia is a frequent feature of poor-prognosis tumours, and the identification of common in vivo hypoxia-related genes is desirable both for prognostic stratification of patients, and development of novel therapies. Although prognostic markers of hypoxia have been identified, there are discrepancies between studies and powerful methods used in large-meta analyses are needed to define generally applicable signatures. A method is described for defining a hypoxia signature that combines previous knowledge derived from in vitro experiments, with co-expression data produced from in vivo samples. We demonstrate that by constructing a gene expression network and then extracting core ‘hub’ (high connectivity) genes it is possible to define signatures that are significantly enriched for phenotype-specific genes, and pathways. While we have used this method to derive a compact and clinically relevant signature of hypoxia in cancer, the approach is likely to have broader applicability.

Specifically, we used the described method in a meta-analysis of a total of 1136 HNSCC and BCs to derive tissue-specific and common signatures of hypoxia by including only genes that are consistently useful across multiple experiments or tissue types respectively. The ability of the method to derive highly prognostic hypoxia signatures despite differences between datasets highlights its robustness.

The gene expression network used to construct the signature was found to be biologically relevant and to map to a discrete set of biochemical pathways, that is significantly enriched for hypoxia-regulated genes and pathways. This finding highlights that not only can in vitro data assist understanding of clinical data, but also the reverse, that clinical data can be used to formulate specific biological hypotheses.

Remarkably, a reduced common hypoxia metagene containing as few as three genes, namely VEGFA, SLC2A1 and PGAM1, was as prognostic as a large signature in independent BC and HNSCC series. Furthermore, it was more prognostic than several published signatures when tested in a set of independent datasets, suggesting a level of general applicability. Specifically, genes with highest connectivity were also the most prognostic across a panel of cancers. This further validates the method, as prognosis was not used to select genes which were only ranked by their connectivity; and this ranking was derived in independent datasets. Although a reduced signature was prognostic in all tumour sites tested, the number of genes before convergence was lower in HNSCC and BC than lung cancer. This offers another positive control as this was a common signature between HNSCC and BC, thus it is expected to reflect their biology to a better extent; however, it also indicates a degree of tumour specificity. The common signature and the tumour-type specific signatures are being evaluated in prospective prognostic and predictive studies in HNSCC and breast cancer.

In summary, this study uses knowledge from in vitro experiments regarding function of multiple genes combined with in vivo co-expression patterns to derive a common hypoxia metagene in multiple cancers that is highly prognostic, whilst being compact and robust.

TABLE 1
Datasets used to train and validate the hypoxia signature

| | | | |
|---|---|---|---|
| Name | Size | Site | Reference |

Training datasets

| | | | |
|---|---|---|---|
| Vice125 | 59 | HN | (Winter et al, 2007) |
| GSE2379 | 20 | HN | (Cromer et al, 2004) |
| GSE6791 | 42 | HN | (Pyeon et al, 2007) |
| GSE6532Oxf | 149 | Breast | (Loi et al, 2008) |
| GSE6532KI | 178 | Breast | (Loi et al, 2008) |
| GSE6532GUY | 87 | Breast | (Loi et al, 2008) |
| GSE2034 | 286 | Breast | (Carroll et al, 2006) |
| GSE3494 | 315 | Breast | (Miller et al, 2005) |

Validation datasets

| | | | |
|---|---|---|---|
| NKI | 295 | Breast | (van de Vijver et al, 2002) |
| Beer | 86 | Lung | (Beer et al, 2002) |
| GSE4573 | 130 | Lung | (Raponi et al, 2006) |
| Chung | 60 | HN | (Chung et al, 2004) |

TABLE 2
Top-ranked genes of the common hypoxia metagene.

| | | | Breast | HNSCC | Common |
|---|---|---|---|---|---|
| HGNC | | | Ranked | Ranked | Score |
| Symbol | Names | Pathway [Source] | Score | Score | (IIC) |
| VEGFA | vascular endothelial | VEGF signaling [KEGG] | 0.99 | 0.99 | 0.98 |
| | growth factor A | | | | |
| SLC2A1 | solute carrier family 2, | Adipocytokine signaling | 0.99 | 0.98 | 0.97 |
| | member 1 | [KEGG] | | | |
| PGAM1 | phosphoglycerate mutase | Glycolysis/Gluconeogenesis | 0.96 | 1.00 | 0.96 |
| | 1 | [KEGG] | | | |
| ENO1 | enolase 1 | Glycolysis/Gluconeogenesis | 0.97 | 0.98 | 0.95 |
| | | [KEGG] | | | |
| LDHA | lactate dehydrogenase A | Glycolysis/Gluconeogenesis | 0.94 | 1.00 | 0.93 |
| | | [KEGG] | | | |
| TPI1 | triosephosphate isomerase | Glycolysis/Gluconeogenesis | 0.92 | 0.99 | 0.91 |
| | 1 | [KEGG] | | | |
| P4HA1 | prolyl 4-hydroxylase, | Arginine and proline | 0.83 | 1.00 | 0.83 |
| | alpha polypeptide I | metabolism [KEGG] | | | |
| MRPS17 | mitochondrial ribosomal | Transport [GO: 0006810] | 0.84 | 0.97 | 0.82 |
| | protein S17 | | | | |
| CDKN3 | cyclin-dependent kinase | G1/S transition of mitotic cell | 0.85 | 0.95 | 0.81 |
| | inhibitor 3 | cycle [GO: 0000082] | | | |
| ADM | adrenomedullin | signal transduction | 0.74 | 1.00 | 0.74 |
| | | [GO: 0007165] | | | |
| NDRG1 | N-myc downstream regulated | response to metal ion | 0.71 | 0.99 | 0.71 |
| | 1 | [GO: 0010038] | | | |
| TUBB6 | tubulin, beta 6 | Gap junction [KEGG] | 0.85 | 0.84 | 0.71 |
| ALDOA | aldolase A, fructose- | Glycolysis/Gluconeogenesis | 0.86 | 0.80 | 0.69 |
| | bisphosphate | [KEGG] | | | |
| MIF | macrophage migration | Tyrosine metabolism [KEGG] | 0.71 | 0.93 | 0.66 |
| | inhibitory factor | | | | |
| ACOT7 | acyl-CoA thioesterase 7 | Lipid Metabolism [KEGG] | 0.73 | 0.89 | 0.65 |

TABLE 3
Prognostic significance of the common hypoxia metagene (CHM)
versus other hypoxia signatures

| | Endpoint & | In-vitro | HN | | | | |
|---|---|---|---|---|---|---|---|
| | significant | Hypoxia | Hypoxia | | | | |
| | clinical | Signature | Metagene | | | | Reduced^{£} |
| Data | covariates | (Chi et al, | (Winter et | Initial | PCA | CHM | CHM |
| (Table 1) | (Cov.)^{&} | 2006) | al, 2007) | Seeds^{μ} | score* | 51genes | k genes |
| NKI | Endpoint: | 2.94 | 3.58 | 2.41 | 3.22 | 4.15 | 5.58 |
| | MFS | [1.39, | [1.53, | [1.05, 5.53] | [1.37, | [1.73, | [2.41, 12.90] |
| | Cov.: Age, T | 6.23] | 8.39] | p = 0.038 | 7.56] | 9.96] | p < 0.001, |
| | Size, Nodal | p = 0.005 | p = 0.003 | | p = 0.007 | p = 0.002 | k = 3 |
| | Status, | | | | | | |
| | Grade, Adj. | | | | | | |
| | Treatment | | | | | | |
| GSE2034^{δ} | Endpoint: | 2.20 | 1.92 | 2.36 | 1.98 | 3.22 | 4.15 |
| | RFS | [1.11, | [0.97, | [0.95, | [1.01, | [1.63, 6.35] | [2.10, 8.18] |
| | Cov.: NA | 4.34] | 3.78] | 3.77] | 3.90] | p = 0.001 | p < 0.001, |
| | | p = 0.024 | p = 0.061 | p = 0.014 | p = 0.048 | | k = 10 |
| GSE3494^{δ} | Endpoint: | 1.19 | 2.07 | 2.87 | 3.61 | 3.16 | 4.27 |
| | DSS | [0.45, | [0.77, | [1.25, | [1.33, | [1.05, 9.53] | [1.53, 11.94] |
| | Cov.: ER, | 3.13] | 5.53] | 4.49] | 9.82] | p = 0.042 | p = 0.006, |
| | PgR, Tumour | p = 0.732 | p = 0.149 | p = 0.029 | p = 0.012 | | k = 2 |
| | size, Nodal | | | | | | |
| | Status | | | | | | |
| Chung | Endpoint: | 3.06 | 14.83 | 6.71 | 1.25 | 6.25 | 34.66 |
| | RFS | [0.53, | [1.8, 122.4] | [0.93, | [0.14, 11.4] | [0.83, | [4.26, 281.95] |
| | Cov.: Intrinsic | 17.6] | p = 0.012 | 48.4] | p = 0.840 | 47.2] | p = 0.001, |
| | sign., | p = 0.210 | | p = 0.059 | | p = 0.077 | k = 2 |
| | differentiation, | | | | | | |
| | batch(strata) | | | | | | |
| Beer | Endpoint: OS | 2.59 | 6.90 | 3.98 | 3.45 | 12.84 | 24.57 |
| | Cov.: Stage | [1.59, 4.2] | [1.34, | [0.72, | [0.59, 20.0] | [1.71, | [2.83, 213.36] |
| | | p = 0.829 | 35.6] | 22.0] | p = 0.168 | 96.5] | p = 0.004, |
| | | | p = 0.021 | p = 0.114 | | p = 0.014 | k = 23 |
| GSE4573 | Endpoint: OS | 3.15 | 1.49 | 2.31 | 1.61 | 2.75 | 2.90 |
| | Cov.: Nodal | [1.32, | [0.65, | [0.93, | [1.14, 2.3] | [1.15, 6.56] | [1.27, 6.61] |
| | Status | 7.54] | 3.43] | 5.72] | p = 0.035 | p = 0.023 | p = 0.012, |
| | | p = 0.010 | p = 0.350 | p = 0.070 | | | k = 38 |

^{&}Reduced models of clinical covariates are derived using backward stepwise likelihood. Signature scores are entered into the reduced model; hazard-ratio, 95% confidence limits and significance (model with and without the signature) are shown.
MFS = Metastases-free survival,
RFS = Recurrence-free surv.,
DSS = Disease-specific surv.,
OS = Overall surv.,
ER/PgR = Estrogen/Progresteron receptor.
^{£}At convergence in the cumulative forest plots.
^{δ}These two datasets were used to develop the signature but no training on outcome was done.
^{μ}Summary score, E, is calculated for the signature including only the initial seeds.
*Score obtained using Principal Components Analysis (Suppl. Methods)

### Example 2

### Metagene Sets

Common Steps for the Head and Neck and Breast Cancer Signatures:

1) Pre-Processing of Array Data:

Data were normalized using gcrma in Bioconductor (http://www.bioconductor or 0 and log 2 expression was considerd.

2) Annotation

The NBC! database, BiomaRt and Matchminer were used to retrieve other aliases and previous IDs for the seeds.

3) Filtering

Filtering was performed based on expression levels and coefficient of variation:—gene were selected for the clustering if their expression level was above the 0.55 quantile, and their coefficient of variation was above the 0.10 quantile, of the global array distribution for expression and CV respectively. To avoid noise arising from cross-contamination in some of the arrays; filtering of unspecific probestes was done using array information provided by Affymetrix. Specifically, probesets with termination x at in the U133 plus2 array, and probesets with termination s at and g at in the U95 arrays, were not used to calculate the seeds' expression levels (for definition of “seed” see clustering section below).

4) Selection of Seeds:

10 genes known to be related to hypoxia in previous studies were used as seeds. Set A in the table below was used in this study:

TABLE 4

| Gene Symbol | Long Name | Ensembl | KEGG |
|---|---|---|---|
| ADM | adrenomedullin | ENSG00000148926 | |
| AK3L1 | adenylate kinase 3-like 1 | ENSG00000162433 | hsa00230 Purine metabolism |
| BNIP3 | BCL2/adenovirus E1B 19 kDa | ENSG00000176171 | |
| | interacting protein 3 | | |
| CA9 | carbonic anhydrase IX | ENSG00000107159 | hsa00910 Nitrogen metabolism |
| ENO1 | enolase 1, (alpha) | ENSG00000074800 | hsa00010 Glycolysis/ |
| | | | Gluconeogenesis |
| HK2 | hexokinase 2 | ENSG00000159399 | hsa00010 Glycolysis/ |
| | | | Gluconeogenesis |
| LDHA | lactate dehydrogenase A | ENSG00000134333 | hsa00010 Glycolysis/ |
| | | | Gluconeogenesis |
| PGK1 | phosphoglycerate kinase 1 | ENSG00000102144 | hsa00010 Glycolysis/ |
| | | | Gluconeogenesis |
| SLC2A1 | solute carrier family 2 (facilitated | ENSG00000117394 | hsa04920 Adipocytokine |
| | glucose transporter), member 1 | | signaling pathway |
| VEGFA | vascular endothelial growth factor A | ENSG00000112715 | |

When more than one probeset mapped to the same gene, the ‘best candidate’ probeset was used:—after filtering was performed to select highly expressed probesets that showed significant variation (see 5 above); a ‘best candidate’ seed was selected as the seed on which most evidence have been accumulated in previous studies; in this case, CA9 was selected as the “gold”-candidate seed. The median expression was computed for this seed if more than one probesets are present (in the case of CA9 only 1 probeset present on the array); for the other seeds, the probeset with expression showing the highest correlation to the expression of the “gold”-candidate seed was selected.

5) Seed Clustering:

The process begins with k seed genes, Π={π₁, π₂ . . . π_{K}} (‘gene’ is used throughout for convenience, although ‘transcript’ is generally more accurate). Spearman correlation, ρ, is computed between seeds and genes Y={y₁, y₂ . . . yₘ} in a dataset of n samples, X={x₁, x₂ . . . xₙ}. For each seed/gene pair, their ‘affinity’ is defined as:

$$δ ( {π}_{i} , {y}_{i} ) = {[ 1 + {e}^{\frac{( {ϑ}_{t} - {{ρ}_{{π}_{i} , {y}_{j}}}^{2} )}{{ϑ}_{s}}} ]}^{- 1} \qquad ( Equation 1 )$$

where θₜ and θₛ define extent and sharpness of the cluster. When θₛ→0, δ reduces to the step function with δ=0 if ρ²<ηₜ, δ=1 if ρ²>θₜ. This was the limit used for this study as it is parameter-free. This needs to be corrected for multiple testing to account for the size of Y; here, α=0.05 after Bonferroni correction was considered. Finally, a membership function is defined:

γ(yᵢ,πₖ)=δ(yᵢ,πₖ)/Σⱼ₌₁^{K}δ(yᵢ,πⱼ) (Equation 2)

An increasing γ indicates stronger membership of a gene to a seed cluster.

6) Shared Neighborhood

The shared neighborhood, S, between two seeds is defined as:

$$S ( {π}_{i} , {π}_{j} ) = \frac{{\sum }_{k = 1 ; k \neq i , j}^{m} min [ γ ( {π}_{i} , {y}_{k} ) , γ ( {π}_{j} , {y}_{k} ) ]}{{\sum }_{k = 1 ; k \neq i , j}^{m} max [ γ ( {π}_{i} , {y}_{k} ) , γ ( {π}_{j} , {y}_{k} ) ]} \qquad ( Equation 3 )$$

where γ is the membership (Eq. 2). Two seeds are considered to carry a high degree of related information if their clusters share many genes (high S values). A sign function is also defined:

$$F ( {π}_{i} , {π}_{j} ) = \frac{\begin{matrix}{\sum }_{k = 1 ; k \neq i , j}^{m} min [ γ ( {π}_{i} , {y}_{k} ) , γ ( {π}_{j} , {y}_{k} ) ] \cdot \\ sgn [ ρ ( {π}_{i} , {y}_{k} ) \cdot ρ ( {π}_{j} , {y}_{k} ) ]\end{matrix}}{{\sum }_{k = 1 ; k \neq i , j}^{m} min [ γ ( {π}_{i} , {y}_{k} ) , γ ( {π}_{j} , {y}_{k} ) ]} \qquad ( Equation 4 )$$

where sgn(x) is the sign function:—sgn(x)=1 if x>0, sgn(x)=−1 if x<0. If two seeds are correlated with their shared features in the same direction, F=1 (seeds are fully concordant); if they are correlated with their shared features in opposite direction, F=−1.

7) Seed-Dependent Connectivity

The strength of the relationship between a gene and the whole set of seeds is estimated using the connectivity function:

$$C ( {y}_{i} ) = \frac{{\sum }_{j = 1 ; j \neq i}^{K} w ( {π}_{j} ) \cdot γ ( {y}_{i} , {π}_{j} )}{{\sum }_{h = 1 ; h \neq i}^{K} w ( {π}_{h} )} \qquad ( Equation 5 )$$

where γ is defined in Eq. 2 and w are weights which regulate the importance of each seed. In this study, we consider w=1, unless yᵢ is one of the seeds, or a probeset biding to the same transcript as the seed; in this case, to avoid bias, for that seed w=0.

A connectivity score, is defined as the fractional rank of C; that is the ranking normalized between 0 (lowest C) and 1 (highest C).

8) Bootstrapping, Monte-Carlo and Meta-Connectivity Score

Random sets of seeds are generated by Monte-Carlo sampling, clusters aggregated around them, C and S calculated. This procedure is repeated to generate null distributions and it provides an estimate of the probability of observing by chance a given value of C and S. Bootstrapping was used to provide best estimates and confidence limits for C and S. These are used in a meta-analysis across several datasets to define a meta-connectivity score as:

$$\hat{C} ( {y}_{i} ) = \frac{{\sum }_{h = 1}^{Nd} {R [ C ( {y}_{i} ) ]}_{h} / {σ}_{h}^{2}}{{\sum }_{h = 1}^{Nd} 1 / {σ}_{h}^{2}} \qquad ( Equation 6 )$$

where R[C(yᵢ)]ₖ is the fractional rank of C (Eq. 5), N_{d} is the number of datasets, σ²ₖ is the variance of the ranked C, R[C(yᵢ)]ₖ, in dataset k for gene yᵢ.

Exactly the same procedure (described above) was applied first to the head and neck datasets and then to the breast cancer datasets. Datasets are listed below:

TABLE 5

| | | | |
|---|---|---|---|
| Name | Size | Site | Reference |

Training datasets

| | | | |
|---|---|---|---|
| Vice125 | 59 | HN | (Winter et al, 2007) |
| GSE2379 | 20 | HN | (Cromer et al, 2004) |
| GSE6791 | 42 | HN | (Pyeon et al, 2007) |
| GSE6532Oxf | 149 | Breast | (Loi et al, 2008) |
| GSE6532KI | 178 | Breast | (Loi et al, 2008) |
| GSE6532GUY | 87 | Breast | (Loi et al, 2008) |
| GSE2034 | 286 | Breast | (Carroll et al, 2006) |
| GSE3494 | 315 | Breast | (Miller et al, 2005) |

Note: The procedure described above was applied in the same way to the head and neck datasets, and then to the breast datasets and two meta-signatures, one in head-and neck, and another in breast were obtained.

The head and neck cancer metagene set, containing the top 100 genes in the HN meta-signature, is shown in the following table:

TABLE 6
Head and neck cancer metagene set:

| Gene | Meta-C |
|---|---|
| PGK1 | 0.993782 |
| AK3L1 | 0.992291 |
| SLC16A1 | 0.991833 |
| SLC2A1 | 0.990579 |
| VEGFA | 0.988468 |
| ENO1 | 0.981204 |
| PGAM1 | 0.962013 |
| BNC1 | 0.955974 |
| CDCA4 | 0.940005 |
| LDHA | 0.936672 |
| HIG2 | 0.929025 |
| TPI1 | 0.918034 |
| CA9 | 0.908603 |
| MAD2L2 | 0.903983 |
| SDC1 | 0.898473 |
| LOC645619 | 0.881414 |
| DCBLD1 | 0.880588 |
| PFKFB4 | 0.876023 |
| ALDOA | 0.862741 |
| FAM83B | 0.857821 |
| GNAI1 | 0.857612 |
| CDKN3 | 0.850681 |
| RRAS2 | 0.849847 |
| ANLN | 0.842485 |
| C20orf20 | 0.841528 |
| MRPS17 | 0.841183 |
| COL4A6 | 0.837064 |
| P4HA1 | 0.834483 |
| PPM1J | 0.825956 |
| KCTD11 | 0.821473 |
| ANGPTL4 | 0.817807 |
| FOSL1 | 0.804235 |
| KRT17 | 0.804072 |
| PYGL | 0.80169 |
| RHOD | 0.797309 |
| TNFRSF12A | 0.792627 |
| FER | 0.7918 |
| ANKRD9 | 0.7868 |
| IGF2BP2 | 0.784355 |
| HSD17B1 | 0.768276 |
| YKT6 | 0.765829 |
| MRPL37 | 0.760842 |
| TGFA | 0.76025 |
| FSCN1 | 0.756417 |
| FAM89A | 0.756049 |
| GAPDH | 0.755969 |
| EREG | 0.752012 |
| KIAA1609 | 0.747641 |
| F2RL1 | 0.74577 |
| ADM | 0.74213 |
| LOC285412 | 0.739965 |
| NDRG1 | 0.737675 |
| RGS20 | 0.735475 |
| TUBB6 | 0.731218 |
| PPARD | 0.728589 |
| ADK | 0.725911 |
| IL1RAP | 0.722424 |
| YWHAG | 0.722278 |
| LRIG2 | 0.716688 |
| EDG7 | 0.712337 |
| CAV2 | 0.711772 |
| MIF | 0.711609 |
| SLC6A10P | 0.709001 |
| TUBA1B | 0.708985 |
| LRRC8E | 0.707163 |
| FUT11 | 0.704768 |
| CDCA8 | 0.694693 |
| C1orf201 | 0.692159 |
| LOC644879 | 0.691203 |
| AP1M2 | 0.690421 |
| TRMT5 | 0.689213 |
| GJB5 | 0.687828 |
| ZDHHC9 | 0.687752 |
| ZNF410 | 0.687644 |
| TIPARP | 0.684208 |
| SMTN | 0.684122 |
| CBLC | 0.684108 |
| EGLN3 | 0.679875 |
| ERO1L | 0.679857 |
| BTBD10 | 0.678293 |
| UBE2V1 | 0.677981 |
| PPIF | 0.677037 |
| B3GNT5 | 0.676941 |
| PPP1R15A | 0.676885 |
| GNPNAT1 | 0.674033 |
| PANX1 | 0.673715 |
| CORO1C | 0.673068 |
| MET | 0.672684 |
| PTHLH | 0.670185 |
| WDR66 | 0.668744 |
| MAGOH | 0.668554 |
| STON2 | 0.667837 |
| ARL4D | 0.667683 |
| SNAPC1 | 0.665042 |
| MCTS1 | 0.66286 |
| EHD2 | 0.661145 |
| RAB38 | 0.660052 |
| GLRX3 | 0.65577 |
| FLJ42117 | 0.654477 |
| TUBA1C | 0.652988 |

The breast cancer metagene set, containing the top 100 genes in the breast cancer meta-signature, is shown in the following table:

TABLE 7
Breast cancer metagene set

| Gene | Meta-C | most representative Affymetrix probeset |
|---|---|---|
| GAPD | 0.997634 | 217398_x_at |
| PGAM1 | 0.997526 | 200886_s_at |
| GARS | 0.996289 | 208693_s_at |
| BNIP3 | 0.995895 | 201849_at |
| LDHA | 0.995872 | 200650_s_at |
| P4HA1 | 0.995708 | 207543_s_at |
| ADM | 0.995046 | 202912_at |
| GPI | 0.994336 | 208308_s_at |
| NDRG1 | 0.993016 | 200632_s_at |
| GAPDH | 0.992841 | AFFX-HUMGAPDH/M33197_3_at |
| DDIT4 | 0.992308 | 202887_s_at |
| VEGF | 0.992186 | 210512_s_at |
| PFKP | 0.991722 | 201037_at |
| TPI1 | 0.990102 | 200822_x_at |
| PGK1 | 0.989769 | 200738_s_at |
| ENO1 | 0.984934 | 201231_s_at |
| DSCR2 | 0.981315 | 203405_at |
| SLC16A3 | 0.981057 | 202856_s_at |
| PRDX4 | 0.979419 | 201923_at |
| CDC20 | 0.97891 | 202870_s_at |
| RRM2 | 0.976834 | 209773_s_at |
| SLC2A1 | 0.97619 | 201250_s_at |
| AK3 | 0.975715 | 225342_at |
| GOLT1B | 0.974507 | 218193_s_at |
| RANBP1 | 0.974015 | 202483_s_at |
| RALA | 0.973974 | 214435_x_at |
| TFRC | 0.973207 | 207332_s_at |
| RIS1 | 0.973049 | 213338_at |
| MCTS1 | 0.971323 | 218163_at |
| SEC61G | 0.969992 | 203484_at |
| ENY2 | 0.969911 | 218482_at |
| MRPS17 | 0.969848 | 218982_s_at |
| MTFR1 | 0.968482 | 203207_s_at |
| MRPL15 | 0.96822 | 218027_at |
| Lrp2bp | 0.967556 | 227337_at |
| CTSL2 | 0.967189 | 210074_at |
| NUP155 | 0.967189 | 206550_s_at |
| SLC7A5 | 0.966302 | 201195_s_at |
| HMGB3 | 0.963721 | 203744_at |
| MMP1 | 0.963559 | 204475_at |
| PSMB5 | 0.963497 | 208799_at |
| DLG7 | 0.963048 | 203764_at |
| BM039 | 0.962249 | 219555_s_at |
| TMEM70 | 0.961161 | 219449_s_at |
| BUB1 | 0.960653 | 209642_at |
| DKFZp762E1312 | 0.960494 | 218726_at |
| IMPAD1 | 0.960314 | 218516_s_at |
| PDIA6 | 0.959873 | 207668_x_at |
| C10orf3 | 0.959509 | 218542_at |
| MRPL13 | 0.959387 | 218049_s_at |
| IL8 | 0.958648 | 202859_x_at |
| CCNB2 | 0.957078 | 202705_at |
| MTCH2 | 0.955381 | 217772_s_at |
| C20orf24 | 0.954747 | 224376_s_at |
| PSMA5 | 0.954502 | 201274_at |
| KIF20A | 0.95432 | 218755_at |
| ATP1B3 | 0.953996 | 208836_at |
| ATP5G3 | 0.953977 | 207507_s_at |
| UBE2S | 0.952806 | 202779_s_at |
| COX4NB | 0.952181 | 218057_x_at |
| RBM35A | 0.95206 | 219121_s_at |
| EIF4EBP1 | 0.951909 | 221539_at |
| TCEB1 | 0.95035 | 202824_s_at |
| NP | 0.950096 | 201695_s_at |
| CCNB1 | 0.950064 | 214710_s_at |
| MELK | 0.948843 | 204825_at |
| CHCHD2 | 0.948816 | 217720_at |
| SF3B5 | 0.948562 | 221263_s_at |
| CDKN3 | 0.947035 | 209714_s_at |
| NUP93 | 0.94703 | 202188_at |
| RNASEH2A | 0.946824 | 203022_at |
| C6orf129 | 0.946508 | 225723_at |
| MAD2L1 | 0.945229 | 203362_s_at |
| LSM4 | 0.944743 | 202736_s_at |
| STK6 | 0.944259 | 204092_s_at |
| IMPA2 | 0.943983 | 203126_at |
| MTHFD2 | 0.943549 | 201761_at |
| TPX2 | 0.942976 | 210052_s_at |
| EIF2S2 | 0.942184 | 208726_s_at |
| NFIL3 | 0.940681 | 203574_at |
| GMPS | 0.940477 | 214431_at |
| PTTG1 | 0.940123 | 203554_x_at |
| SRD5A1 | 0.939546 | 211056_s_at |
| GGH | 0.938966 | 203560_at |
| BTG3 | 0.938627 | 213134_x_at |
| PSMD8 | 0.938397 | 200820_at |
| YEATS2 | 0.936797 | 221203_s_at |
| DC13 | 0.935903 | 218447_at |
| KIF4A | 0.935566 | 218355_at |
| KIF18A | 0.935156 | 221258_s_at |
| KPNA2 | 0.934994 | 211762_s_at |
| OR7E38P | 0.93384 | 217499_x_at |
| PRO1855 | 0.933763 | 222231_s_at |
| HCCS | 0.933171 | 203746_s_at |
| PLOD1 | 0.9331 | 200827_at |
| UBE2A | 0.932799 | 201898_s_at |
| RACGAP1 | 0.931545 | 222077_s_at |
| CDC2 | 0.930715 | 203213_at |
| MIF | 0.93027 | 217871_s_at |
| SHMT2 | 0.928808 | 214437_s_at |

Finally a common hypoxia signature (or common metagene as referred to herein) between head and neck, and breast cancer, was derived by taking the C scores product, EC. This is effectively a rank product, as C is an average rank (Eq. 6).

So the meta-C score for the HN (as calculated by Eq. 6) was multiplied by the meta-C score for the breast cancer signature (as calculated by Eq. 6). The results for this give the common signature which is the common metagene, and which is shown in the following table:

TABLE 8
Common metagene set:

| | Symbol | Symbol | Meta-C for | Meta-C for | Comon C |
|---|---|---|---|---|---|
| Affymetrix | (Affymetrix | (Matchminer | head and neck | breast | score |
| probeset ID | annotation) | annotation) | cancer | cancer | (πC) |
| 210512_s_at | VEGFA | VEGFA | 0.988468 | 0.992186 | 0.980744 |
| 201250_s_at | SLC2A1 | SLC2A1 | 0.990579 | 0.97619 | 0.966993 |
| 200886_s_at | PGAM1 | PGAM1 | 0.962013 | 0.997526 | 0.959633 |
| 201231_s_at | ENO1 | ENO1 | 0.968181 | 0.984934 | 0.953594 |
| 200650_s_at | LDHA | LDHA | 0.936672 | 0.995872 | 0.932806 |
| 200822_x_at | TPI1 | TPI1 | 0.918034 | 0.990102 | 0.908948 |
| 207543_s_at | P4HA1 | P4HA1 | 0.834483 | 0.995708 | 0.830901 |
| 218982_s_at | MRPS17 | MRPS17 | 0.841183 | 0.969848 | 0.81582 |
| 209714_s_at | CDKN3 | CDKN3 | 0.850681 | 0.947035 | 0.805625 |
| 202912_at | ADM | ADM | 0.74213 | 0.995046 | 0.738453 |
| 200632_s_at | NDRG1 | NDRG1 | 0.713339 | 0.993016 | 0.708357 |
| 209191_at | TUBB6 | TUBB6 | 0.846992 | 0.835431 | 0.707603 |
| 238996_x_at | ALDOA | ALDOA | 0.862741 | 0.799858 | 0.69007 |
| 217871_s_at | MIF | MIF | 0.711609 | 0.93027 | 0.661988 |
| 208002_s_at | ACOT7 | ACOT7 | 0.7341 | 0.891762 | 0.654643 |
| 218163_at | MCTS1 | MCTS1 | 0.66286 | 0.971323 | 0.643852 |
| 201896_s_at | PSRC1 | PSRC1 | 0.869886 | 0.734711 | 0.639115 |
| 216088_s_at | PSMA7 | PSMA7 | 0.713358 | 0.88764 | 0.633205 |
| 222608_s_at | ANLN | ANLN | 0.842485 | 0.747685 | 0.629914 |
| 212639_x_at | K-ALPHA-1 | TUBA1B | 0.708985 | 0.879883 | 0.623824 |
| 223234_at | MAD2L2 | MAD2L2 | 0.903983 | 0.678934 | 0.613745 |
| 208308_s_at | GPI | GPI | 0.592527 | 0.994336 | 0.589171 |
| 209251_x_at | TUBA6 | TUBA1C | 0.652988 | 0.900391 | 0.587944 |
| 217943_s_at | RPRC1 | MAP7D1 | 0.803124 | 0.717636 | 0.576351 |
| 202887_s_at | DDIT4 | DDIT4 | 0.572277 | 0.992308 | 0.567875 |
| 201849_at | BNIP3 | BNIP3 | 0.554323 | 0.995895 | 0.552048 |
| 218586_at | C20orf20 | C20orf20 | 0.841528 | 0.651867 | 0.548565 |
| 218507_at | HIG2 | HIG2 | 0.929025 | 0.589453 | 0.547617 |
| 217398_x_at | GAPD | GAPDH | 0.547008 | 0.997634 | 0.545714 |
| 218049_s_at | MRPL13 | MRPL13 | 0.567857 | 0.959387 | 0.544794 |
| 217720_at | CHCHD2 | CHCHD2 | 0.573503 | 0.948816 | 0.544149 |
| 217785_s_at | YKT6 | YKT6 | 0.765829 | 0.702477 | 0.537978 |
| 201695_s_at | NP | NP | 0.566221 | 0.950096 | 0.537964 |
| 221676_s_at | CORO1C | CORO1C | 0.615699 | 0.86939 | 0.535283 |
| 203484_at | SEC61G | SEC61G | 0.546356 | 0.969992 | 0.529961 |
| 227337_at | Lrp2bp | ANKRD37 | 0.542026 | 0.967556 | 0.52444 |
| 219121_s_at | RBM35A | RBM35A | 0.547712 | 0.95206 | 0.521455 |
| 201037_at | PFKP | PFKP | 0.52543 | 0.991722 | 0.52108 |
| 219493_at | SHCBP1 | SHCBP1 | 0.578941 | 0.892156 | 0.516506 |
| 210074_at | CTSL2 | CTSL2 | 0.531612 | 0.967189 | 0.514169 |
| 218755_at | KIF20A | KIF20A | 0.537673 | 0.95432 | 0.513112 |
| 221020_s_at | MFTC | SLC25A32 | 0.601887 | 0.847949 | 0.51037 |
| 218235_s_at | UTP11L | UTP11L | 0.736755 | 0.692208 | 0.509987 |
| 202235_at | SLC16A1 | SLC16A1 | 0.988372 | 0.514066 | 0.508088 |
| 218027_at | MRPL15 | MRPL15 | 0.520842 | 0.96822 | 0.50429 |
| 218355_at | KIF4A | KIF4A | 0.538833 | 0.935566 | 0.504114 |
| 215084_s_at | LRRC42 | LRRC42 | 0.647353 | 0.77307 | 0.500449 |

Prognostic Validation

To check if a reduced signature was as prognostic as a full signature we used cumulative forest plots based on connectivity score—this was not used to train the signatures but just to understand their performance as prognostic markers in independent datasets.

A summary expression score, E, is defined in each sample as the median of the absolute expression of the genes in the signature. The median is used as summary statistics to reduce the effect of outliers. A cumulative forest plot is defined:—genes are added to the signature, one by one, in order of their connectivity, C, score so that genes that are introduced first have the highest connectivity. At each step, a summary expression, E, is derived using the new gene and genes from the previous steps. Samples are then ranked by their E value; this assigns a hypoxia score (HS) from lowest (least hypoxic) to highest (most hypoxic). HS is then renormalized between 0 and 1; introduced into a Cox multivariate analysis that includes the other significant clinical covariates; and the hazard ratio (HR) of the HS is calculated.

Prognostic validation (without further training): This was applied in the same way to the HN, BC and common signatures. Results for these validations are provided in Example 1 table 3 for the common signature; and in the supplementary table S4 for the HN and BC meta-signatures.

Selection of the genes for the PCR cards:

A refined and reduced signature of 26 genes was selected for the development of a PCR card for use to assess a hypoxia phenotype of a tumour.

After the bioinformatics derivation described above (points 1-8) more practical filters were applied to the meta-HN signature to select genes which would go on a preferred PCR card to be validated prospectically:

Top 26 genes from the above meta-analysis (highest meta-C score as calculated by Eq. 5, and as given the head and neck metagene set) which also fulfilled:
showed a log₂ fold change >0.4 in a small subsets of 5 high and 5 low hypoxia score HN patients (this hypoxia score was based on our first publication in cancer research, Winter et al, 2007)
were also present in at least two datasets in the meta-analysis
sufficiently adequate performance in PCR experiments

If one of the top 26 genes was found not to fulfill these criteria, the next one down in order of meta-C score was selected and so on until 26 genes were selected that fulfilled all of the above. This gave the preferred 26-gene set shown in the following table:

TABLE 9
26-gene set:

PGK1
SLC16A1
SLC2A1
VEGFA
ENO1
PGAM1
BNC1
KRT17
LDHA
TPI1
CA9
SDC1
DCBLD1
ALDOA
FAM83B
GNAI1
CDKN3
ANLN
C20orf20
MRPS17
COL4A6
P4HA1
PPM1J^{†}
KCTD11
ANGPTL4
FOSL1
^{†}In some cases in accordance with the present invention, PPM1J may be replaced by HIG2.

TABLE 10

| | | | |
|---|---|---|---|
| SEQ ID NO | Gene name | RefSeq | GI |

Hypoxia-related Genes

| | | | |
|---|---|---|---|
| 1 | SLC2A1 | NM_006516.2 | GI:166795298 |
| 2 | VEGFA | NM_003376.5 | GI:284172448 |
| 3 | | NM_001025366.2 | GI:284172447 |
| 4 | | NM_001025367.2 | GI:284172449 |
| 5 | | NM_001025368.2 | GI:284172452 |
| 6 | | NM_001171626.1 | GI:284172464 |
| 7 | | NM_001171625.1 | GI:284172462 |
| 8 | | NM_001171624.1 | GI:284172460 |
| 9 | | NM_001171623.1 | GI:284172458 |
| 10 | PGAM1 | NM_002629.2 | GI:31543395 |
| 11 | PGK1 | NM_000291.3 | GI:183603937 |
| 12 | SLC16A1 | NM_003051.3 | GI:115583684 |
| 13 | | NM_001166496.1 | GI:262073006 |
| 14 | ENO1 | NM_001428.2 | GI:16507965 |
| 15 | BNC1 | NM_001717.3 | GI:157276587 |
| 16 | KRT17 | NM_000422.2 | GI:197383031 |
| 17 | LDHA | NM_001135239.1 | GI:207028493 |
| 18 | | NM_001165414.1 | GI:260099722 |
| 19 | | NM_001165415.1 | GI:260099724 |
| 20 | | NM_001165416.1 | GI:260099726 |
| 21 | | NM_028500.1 | GI:260099728 |
| 22 | | NM_005566.3 | GI:207028465 |
| 23 | TPI1 | NM_001159287.1 | GI:226529916 |
| 24 | | NM_027483.1 | GI:226529936 |
| 25 | | NM_000365.5 | GI:226529872 |
| 26 | CA9 | NM_001216.2 | GI:169636419 |
| 27 | SDC1 | NM_001006946.1 | GI:55749479 |
| 28 | | NM_002997.4 | GI:55925657 |
| 29 | DCBLD1 | NM_173674.1 | GI:27735142 |
| 30 | ALDOA | NM_184041.1 | GI:34577109 |
| 31 | | NM_184043.1 | GI:34577111 |
| 32 | | NM_001127617.1 | GI:193794813 |
| 33 | | NM_000034.2 | GI:34577108 |
| 34 | FAM83B | NM_001010872.1 | GI:61676088 |
| 35 | GNAI1 | NM_002069.5 | GI:156071490 |
| 36 | CDKN3 | NM_005192.3 | GI:195927023 |
| 37 | | NM_001130851.1 | GI:195927024 |
| 38 | ANLN | NM_018685.2 | GI:31657093 |
| 39 | C20orf20 | NM_018270.4 | GI:209413768 |
| 40 | MRPS17 | NM_015969.2 | GI:16554613 |
| 41 | COL4A6 | NM_001847.2 | GI:148536822 |
| 42 | | NM_033641.2 | GI:148536826 |
| 43 | P4HA1 | NM_001017962.2 | GI:217272847 |
| 44 | | NM_001142595.1 | GI:217272848 |
| 45 | | NM_001142596.1 | GI:217272850 |
| 46 | | NM_000917.3 | GI:217272856 |
| 47 | HIG2 | NM_013332.3 | GI:149192860 |
| 48 | KCTD11 | NM_001002914.2 | GI:146149101 |
| 49 | ANGPTL4 | NM_001039667.1 | GI:89264695 |
| 50 | | NM_139314.1 | GI:21536397 |
| 51 | FOSL1 | NM_005438.3 | GI:156071499 |
| 52 | PPM1J | NM_005167.5 | GI:65506327 |

Control Genes

| | | | |
|---|---|---|---|
| 53 | GNB2L1 | NM_006098.4 | GI:83641897 |
| 54 | B2M | NM_004048.2 | GI:37704380 |
| 55 | RPL11 | NM_000975.2 | GI:15431289 |
| 56 | RPL24 | NM_000986.3 | GI:78190466 |
| 57 | HPRT1 | NM_000194.2 | GI:164518913 |

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

1. MacKay, R. I., Niemierko, A., Goitein, M. & Hendry, J. H. Potential clinical impact of normal-tissue intrinsic radiosensitivity testing. Radiother Oncol 46, 215-6 (1998).
2. Swedish Council on Technology Assessment in Health Care (SBU). Radiotherapy for Cancer. Acta Oncol 35 Suppl 6, 1-100 (1996).
3. Lundgren K, Holm C, Landberg G. Hypoxia and breast cancer: prognostic and therapeutic implications. Cell Mol Life Sci 2007 [Epub ahead of print].
4. Brizel D M, Rosner G L, Prosnitz L R, Dewhirst M W. Patterns and variability of tumour oxygenation in human soft tissue sarcomas, cervical carcinomas, and lymph node metastases. Int J Radiat Oncol Biol Phys 1995; 32(4):1121-5.
5. Vaupel P, Hockel M, Mayer A. Detection and characterization of tumour hypoxia using p02 histography. Antioxid Redox Signal 2007; 9(8):1221-35.
6. Vaupel P, Okunieff P, Neuringer L J. Blood flow, tissue oxygenation, pH distribution, and energy metabolism of murine mammary adenocarcinomas during growth. Adv Exp Med Biol 1989; 248:835-45.
7. Vaupel P, Schlenger K, Knoop C, Hockel M. Oxygenation of human tumours: evaluation of tissue oxygen distribution in breast cancers by computerized O2 tension measurements. Cancer Res 1991; 51(12):3316-22.
8. Dewhirst M W. Intermittent hypoxia furthers the rationale for hypoxia-inducible factor-1 targeting. Cancer Res 2007; 67(3):854-5.
9. Rzymski T, Harris A L. The unfolded protein response and integrated stress response to anoxia. Clin Cancer Res 2007; 13(9):2537-40.
10. Harris A L. Hypoxia—a key regulatory factor in tumour growth. Nat Rev Cancer 2002; 2(1):38-47.
11. Maynard M A, Ohh M. The role of hypoxia-inducible factors in cancer. Cell Mol Life Sci 2007; 64(16):2170-80.
12. Patiar S, Harris A L. Role of hypoxia-inducible factor-1alpha as a cancer therapy target. Endocr Relat Cancer 2006; 13(Suppl. 1): S61-75.
13. Schofield C J, Ratcliffe P J. Oxygen sensing by HIF hydroxylases. Nat Rev Mol Cell Biol 2004; 5(5):343-54.
14. Knowles H J, Raval R R, Harris A L, Ratcliffe P J. Effect of ascorbate on the activity of hypoxia-inducible factor in cancer cells. Cancer Res 2003; 63(8):1764-8.
15. Tan E Y, Campo L, Han C, et al. Cytoplasmic location of factor inhibiting-HIF (FIH)-1 is associated with an enhanced hypoxic response and a shorter survival in invasive breast cancer. Breast Cancer Res 2007; 9(6):R89.
16. Vleugel M M, Greijer A E, Shvarts A, et al. Differential prognostic impact of hypoxia induced and diffuse HIF-1alpha expression in invasive breast cancer. J Clin Pathol 2005; 58(2): 172-7.
17. Turashvili G, Bouchal J, Burkadze G, Kolar Z. Wnt signalling pathway in mammary gland development and carcinogenesis. Pathobiology 2006; 73(5):213-23.
18. Novak A, Hsu S C, Leung-Hagesteijn C, et al. Cell adhesion and the integrin-linked kinase regulate the LEF-1 and betacatenin signaling pathways. Proc Natl Acad Sci USA 1998; 95(8):4374-9.
19. Eger A, Stockinger A, Schaffhauser B, Beug H, Foisner R. Epithelial mesenchymal transition by c-Fos estrogen receptor activation involves nuclear translocation of beta-catenin and upregulation of beta-catenin/lymphoid enhancer binding factor-1 transcriptional activity. J Cell Biol 2000; 148(1):173-88.
20. Krishnamachary B, Berg-Dixon S, Kelly B, et al. Regulation of colon carcinoma cell invasion by hypoxia-inducible factor 1. Cancer Res 2003; 63(5):1138-43.
21. Luo Y, He D L, Ning L, Shen S L, Li L, Li X. Hypoxia-inducible factor-1alpha induces the epithelial-mesenchymal transition of human prostatecancer cells. Chin Med J (Engl) 2006; 119(9):713-8.
22. Jiang Y G, Luo Y, He D L, et al. Role of Wnt/beta-catenin signalling pathway in epithelial-mesenchymal transition of human prostate cancer induced by hypoxia-inducible factor-1alpha. Int J Urol 2007; 14(11):1034-9.
23. Shuin T, Kondo K, Ashida S, et al. Germline and somatic mutations in von Hippel-Lindau disease gene and its significance in the development of kidney cancer. Contrib Nephrol 1999; 128:1-10.
24. Shuin T, Kondo K, Torigoe S, et al. Frequent somatic mutations and loss of heterozygosity of the von Hippel-Lindau tumour suppressor gene in primary human renal cell carcinomas. Cancer Res 1994; 54(11):2852-5.
25. Zundel W, Schindler C, Haas-Kogan D, et al. Loss of PTEN facilitates HIF-1-mediated gene expression. Genes Dev 2000; 14(4):391-6.
26. Grover-McKay M, Walsh S A, Seftor E A, Thomas P A, Hendrix M J. Role for glucose transporter 1 protein in human breast cancer. Pathol Oncol Res 1998; 4(2):115-20.
27. Semenza G L. Life with oxygen. Science 2007; 318(5847):62-4.
28. Prabhakar N R, Kumar G K, Nanduri J, Semenza G L. ROS signaling in systemic and cellular responses to chronic intermittent hypoxia. Antioxid Redox Signal 2007; 9(9): 1397-403.
29. Semenza G L. Oxygen-dependent regulation of mitochondrial respiration by hypoxia-inducible factor 1. Biochem J 2007; 405(1):1-9.
30. Wykoff C C, Beasley N J, Watson P H, et al. Hypoxia-inducible expression of tumour-associated carbonic anhydrases. Cancer Res 2000; 60(24):7075-83.
31. Generali D, Fox S B, Berruti A, et al. Role of carbonic anhydrase IX expression in prediction of the efficacy and outcome of primary epirubicin/tamoxifen therapy for breast cancer. Endocr Relat Cancer 2006; 13(3):921-30.
32. Kaufman B, Scharf O, Arbeit J, et al. Proceedings of the Oxygen Homeostasis/Hypoxia Meeting. Cancer Res 2004; 64(9):3350-6.
33. Hanahan D, Folkman J. Patterns and emerging mechanisms of the angiogenic switch during tumourigenesis. Cell 1996; 86(3):353-64.
34. Weidner N, Semple J P, Welch W R, Folkman J. Tumour angiogenesis and metastasis-correlation in invasive breast carcinoma. N Engl J Med 1991; 324(1):1-8.
35. Ferrara N. Vascular endothelial growth factor: basic science and clinical progress. Endocr Rev 2004; 25(4):581-611.
36. Tischer E, Mitchell R, Hartman T, et al. The human gene for vascular endothelial growth factor. Multiple protein forms are encoded through alternative exon splicing. J Biol Chem 1991; 266(18):11947-54.
37. Cao Y, Li C Y, Moeller B J, et al. Observation of incipient tumour angiogenesis that is independent of hypoxia and hypoxia inducible factor-1 activation. Cancer Res 2005; 65(13):5498-505.
38. Zhou J, Schmid T, Brune B. Tumour necrosis factor-alpha causes accumulation of a ubiquitinated form of hypoxia inducible factor-1alpha through a nuclear factor-kappaBdependent pathway. Mol Biol Cell 2003; 14(6):2216-25.
39. Sainson R C, Harris A L. Hypoxia-regulated differentiation: let's step it up a Notch. Trends Mol Med 2006; 12(4):141-3.
40. Riesterer O, Milas L, Ang K K. Use of molecular biomarkers for predicting the response to radiotherapy with or without chemotherapy. J Clin Oncol 2007; 25(26):4075-83.
41. Durand R E. The influence of microenvironmental factors during cancer therapy. In Vivo 1994; 8(5):691-702.
42. Teicher B A. Hypoxia and drug resistance. Cancer Metastasis Rev 1994; 13(2):139-68.
43. Nordsmark M, Bentzen S M, Rudat V, et al. Prognostic value of tumour oxygenation in 397 head and neck tumours after primary radiation therapy. An international multi-center study. Radiother Oncol 2005; 77:18-24.
44. Koukourakis M I, Giatromanolaki A, Sivridis E, et al. Hypoxia-regulated carbonic anhydrase-9 (CA9) relates to poor vascularization and resistance of squamous cell head and neck cancer to chemoradiotherapy. Clin Cancer Res 2001; 7:3399-403.
45. Koukourakis M I, Giatromanolaki A, Sivridis E, et al. Hypoxia-inducible factor (HIF1A and HIF2A), angiogenesis, and chemoradiotherapy outcome of squamous cell head-and-neck cancer. Int J Radiat Oncol Biol Phys 2002; 53:1192-202.
46. Aebersold D M, Burri P, Beer K T, et al. Expression of hypoxia-inducible factor-1α: a novel predictive and prognostic parameter in the radiotherapy of oropharyngeal cancer. Cancer Res 2001; 61:2911-6.
47. Swinson D E, Jones J L, Richardson D, et al. Carbonic anhydrase IX expression, a novel surrogate marker of tumour hypoxia, is associated with a poor prognosis in non-small-cell lung cancer. J Clin Oncol 2003; 21:473-82.
48. Giatromanolaki A, Koukourakis M I, Sivridis E, et al. Relation of hypoxia inducible factor 1α and 2α in operable non-small cell lung cancer to angiogenic/molecular profile of tumours and survival. Br J Cancer 2001; 85:881-90.
49. Hui E P, Chan A T, Pezzella F, et al. Coexpression of hypoxia-inducible factors 1α and 2α, carbonic anhydrase IX, and vascular endothelial growth factor in nasopharyngeal carcinoma and relationship to survival. Clin Cancer Res 2002; 8:2595-604.
50. Turner K J, Crew J P, Wykoff C C, et al. The hypoxia-inducible genes VEGF and CA9 are differentially regulated in superficial vs invasive bladder cancer. Br J Cancer 2002; 86:1276-82.
51. Loncaster, J. A. et al. Carbonic anhydrase (CA IX) expression, a potential new intrinsic marker of hypoxia: correlations with tumour oxygen measurements and prognosis in locally advanced carcinoma of the cervix. Cancer Res 61, 6394-9 (2001).
52. Koukourakis, M. I. et al. Hypoxia-inducible factor (HIF1A and HIF2A), angiogenesis, and chemoradiotherapy outcome of squamous cell head-and-neck cancer. Intl Radiat Oncol Biol Phys 53, 1192-202 (2002).
53. Camps, C. et al. hsa-miR-210 Is Induced by Hypoxia and Is an Independent Prognostic Factor in Breast Cancer. Clin Cancer Res 14, 1340-8 (2008).
54. C. H. Chung, P. S. Bernard and C. M. Perou, Molecular portraits and the family tree of cancer, Nat Genet 32 (2002), pp. 533-540.
55. S. Ramaswamy, P. Tamayo and R. Rifkin et al., Multiclass cancer diagnosis using tumour gene expression signatures, Proc Natl Acad Sci USA 98 (2001), pp. 15149-15154.
56 L. D. Miller, J. Smeds and J. George et al., An expression signature for p53 status in human breast cancer predicts mutation status, transcriptional effects, and patient survival, Proc Natl Acad Sci USA 102 (2005), pp. 13550-13555.
57. L. J. van't Veer, H. Dai and M. J. van de Vijver et al., Gene expression profiling predicts clinical outcome of breast cancer, Nature 415 (2002), pp. 530-536.
58. M. J. van de Vijver, Y. D. He and L. J. van't Veer et al., A gene-expression signature as a predictor of survival in breast cancer, N Engl J Med 347 (2002), pp. 1999-2009.
59. A. H. Bild, A. Potti and J. R. Nevins, Linking oncogenic pathways with therapeutic opportunities, Nat Rev Cancer 6 (2006), pp. 735-741.
60. H. Y. Chang, J. B. Sneddon and A. A. Alizadeh et al., Gene expression signature of fibroblast serum response predicts human cancer progression: similarities between tumours and wounds, PLoS Biol 2 (2004), p. E7.
61. J. T. Chi, Z. Wang and D. S. Nuyten et al., Gene expression programs in response to hypoxia: cell type specificity and prognostic significance in human cancers, PLoS Med 3 (2006), p. e47.
62. E. S. Huang, E. P. Black, H. Dressman, M. West and J. R. Nevins, Gene expression phenotypes of oncogenic signaling pathways, Cell Cycle 2 (2003), pp. 415-417.
63. Winter, S. C. et al. Relation of a hypoxia metagene derived from head and neck cancer to prognosis of multiple cancers. Cancer Res 67, 3441-9 (2007).
64. Chung, C. H. et al. Molecular classification of head and neck squamous cell carcinomas using patterns of gene expression. Cancer Cell 5, 489-500 (2004).
65. Chang, H. Y. et al. Robustness, scalability, and integration of a wound-response gene expression signature in predicting breast cancer survival. Proc Natl Acad Sci USA 102, 3738-43 (2005).
66. Jemal A, Siegel R, Ward E, et al. Cancer statistics, 2008. CA: Cancer Journal for Clinicians. 2008; 58(2):71-96.
67. Boring C C, Squires T S, Tong T, Montgomery S. Cancer statistics, 1994. CA Cancer J Clin 1994; 44:7-26.
68. Bernier J, Domenge C, Ozsahin M, et al. Postoperative irradiation with or without concomitant chemotherapy for locally advanced head and neck cancer. N Engl J Med 2004; 350:1945-52.
69. Sessions D G, Spector G J, Lenox J, et al. Analysis of treatment results for oral tongue cancer. Laryngoscope 2002; 112:616-25.
70. Giaccia A J. Hypoxic stress proteins: survival of the fittest. Semin Radiat Oncol 1996; 6:46-58.
71. Wouters B G, Weppler S A, Koritzinsky M, et al. Hypoxia as a target for combined modality treatments. Eur J Cancer 2002; 38:240-57.
72. Semenza G L. Targeting HIF-1 for cancer therapy. Nat Rev Cancer 2003; 3:721-32.
73. P. Vaupel, M. Hockel and A. Mayer, Detection and characterization of tumor hypoxia using p_{O2} histography, Antioxid Redox Signal 9 (8) (2007), pp. 1221-1235.
74. P. L. Olive, J. P. Banath and C. Aquino-Parsons, Measuring hypoxia in solid tumours—is there a gold standard?, Acta Oncol 40 (8) (2001), pp. 917-923.
75. M. W. Dewhirst, Intermittent hypoxia furthers the rationale for hypoxia-inducible factor-1 targeting, Cancer Res 67 (3) (2007), pp. 854-855.
76. J. L. Tatum, G. J. Kelloff and R. J. Gillies et al., Hypoxia: importance in tumor biology, noninvasive measurement by imaging, and value of its measurement in the management of cancer therapy, Int Radiat Biol 82 (10) (2006), pp. 699-757.
77. H. B. Stone, J. M. Brown, T. L. Phillips and R. M. Sutherland, Oxygen in human tumors: correlations between methods of measurement and response to therapy.
Summary of a workshop held Nov. 19-20, 1992, at the National Cancer Institute, Bethesda, Md., Radiat Res 136 (3) (1993), pp. 422-434.
78. E. J. Moon, D. M. Brizel, J. T. Chi and M. W. Dewhirst, The potential role of intrinsic hypoxia markers as prognostic variables in cancer, Antioxid Redox Signal 9 (8) (2007), pp. 1237-1294.
79. Beasley N J, Leek R, Alam M, Turley H, Cox G J, Gatter K, Millard P, Fuggle S, Harris A L, 2002. Hypoxia-inducible factors HIF-1alpha and HIF-2alpha in head and neck cancer: relationship to tumor biology and treatment outcome in surgically resected patients, Cancer Res 62: 2493-2497,
80. Winter S C, Shah K A, Han C, Campo L, Turley H, Leek R, Corbridge R J, Cox G J, Harris A L, 2006. The relation between hypoxia-inducible factor (HIF)-1alpha and HIF-2alpha expression with anemia and outcome in surgically treated head and neck cancer. Cancer 107: 757-766,
81. Vaupel R, Mayer A. 2007. Hypoxia in cancer: Significance and impact on clinical outcome. Cancer Metastisis Rev 26: 225-239.
82, D. Generali, A. Berruti and M. P. Brizzi et al., Hypoxia-inducible factor-1alpha expression predicts a poor response to primary chemoendocrine therapy and disease-free survival in primary human breast cancer, Clin Cancer Res 12 (15) (2006), pp. 4562-4568.
83. J. P. Dales, S. Garcia and S. Meunier-Carpentier et al., Overexpression of hypoxia-inducible factor HIF-1alpha predicts early relapse in breast cancer: retrospective study in a series of 745 patients, Int J Cancer 116 (5) (2005), pp. 734-739.
84. M. Schindl, S. F. Schoppmann and H. Samonigg et al., Overexpression of hypoxia-inducible factor 1alpha is associated with an unfavorable prognosis in lymph node-positive breast cancer, Clin Cancer Res 8 (6) (2002), pp. 1831-1837.
85. R. Bos, P. van der Groep and A. E. Greijer et al., Levels of hypoxia-inducible factor-1alpha independently predict prognosis in patients with lymph node negative breast carcinoma, Cancer 97 (6) (2003), pp. 1573-1581.
86. J. A. Loncaster, A. L. Harris and S. E. Davidson et al., Carbonic anhydrase (CA IX) expression, a potential new intrinsic marker of hypoxia: correlations with tumor oxygen measurements and prognosis in locally advanced carcinoma of the cervix, Cancer Res 61 (17) (2001), pp. 6394-6399.
87. S. K. Chia, C. C. Wykoff and P. H. Watson et al., Prognostic significance of a novel hypoxia-regulated marker, carbonic anhydrase IX, in invasive breast carcinoma, J Clin Oncol 19 (16) (2001), pp. 3660-3668.
88. D. J. Brennan, K. Jirstrom and A. Kronblad et al., CA IX is an independent prognostic marker in premenopausal breast cancer patients with one to three positive lymph nodes and a putative marker of radiation resistance, Clin Cancer Res 12 (21) (2006), pp. 6421-6431.
89. M. Toi, K. Inada, H. Suzuki and T. Tominaga, Tumor angiogenesis in breast cancer: its importance as a prognostic indicator and the association with vascular endothelial growth factor expression, Breast Cancer Res Treat 36 (2) (1995), pp. 193-204.
90. G. Gasparini, M. Toi and M. Gion et al., Prognostic significance of vascular endothelial growth factor protein in node-negative breast carcinoma, J Natl Cancer Inst 89 (2) (1997), pp. 139-147.
91. G. Gasparini, M. Toi and R. Miceli et al., Clinical relevance of vascular endothelial growth factor and thymidine phosphorylase in patients with node-positive breast cancer treated with either adjuvant chemotherapy or hormone therapy, Cancer J Sci Am 5 (2) (1999), pp. 101-111.
92. U. Eppenberger, W. Kueng and J. M. Schlaeppi et al., Markers of tumor angiogenesis and proteolysis independently define high- and low-risk subsets of node-negative breast cancer patients, J Clin Oncol 16 (9) (1998), pp. 3129-3136.
93. L. Yen, X. L. You and A. E. Al Moustafa et al., Heregulin selectively upregulates vascular endothelial growth factor secretion in cancer cells and stimulates angiogenesis, Oncogene 19 (31) (2000), pp. 3460-3469.
94. E. Laughner, P. Taghavi, K. Chiles, P. C. Mahon and G. L. Semenza, HER2 (neu) signaling increases the rate of hypoxia-inducible factor 1alpha (HIF-1alpha) synthesis: novel mechanism for HIF-1-mediated vascular endothelial growth factor expression, Mol Cell Biol 21 (12) (2001), pp. 3995-4004.
95. S. Olewniczak, M. Chosia, A. Kwas, A. Kram and W. Domagala, Angiogenesis and some prognostic parameters of invasive ductal breast carcinoma in women, Pol J Pathol 53 (4) (2002), pp. 183-188.
96. G. Gasparini, Clinical significance of determination of surrogate markers of angiogenesis in breast cancer, Crit Rev Oncol Hematol 37 (2) (2001), pp. 97-114.
97. B. Uzzan, P. Nicolas, M. Cucherat and G. Y. Perret, Microvessel density as a prognostic factor in women with breast cancer: a systematic review of the literature and meta-analysis, Cancer Res 64 (9) (2004), pp. 2941-2955.
98. B. K. Linderholm, B. Lindh and L. Beckman et al., Prognostic correlation of basic fibroblast growth factor and vascular endothelial growth factor in 1307 primary breast cancers, Clin Breast Cancer4 (5) (2003), pp. 340-347.
99. R. Seigneuric, M. H. Starmans and G. Fung et al., Impact of supervised gene signatures of early hypoxia on patient survival, Radiother Oncol 83 (3) (2007), pp. 374-382.
100. Pramana, J. et al. Gene expression profiling to predict outcome after chemoradiation in head and neck cancer. Int J Radiat Oncol Biol Phys 69, 1544-52 (2007).
101. Ein-Dor, L., Kela, I., Getz, G., Givol, D. & Domany, E. Outcome signature genes in breast cancer: is there a unique set? Bioinformatics 21, 171-8 (2005).
102. Shen, R., Ghosh, D. & Chinnaiyan, A.M. Prognostic meta-signature of breast cancer developed by two-stage mixture modeling of microarray data. BMC Genomics 5, 94 (2004).
103. Kaanders, J. H. et al. Pimonidazole binding and tumor vascularity predict for treatment outcome in head and neck cancer. Cancer Res 62, 7066-74 (2002).
104. Kaanders, J. H. et al. ARGON: experience in 215 patients with advanced head-and-neck cancer. Int J Radiat Oncol Biol Phys 52, 769-78 (2002).
105. Overgaard, J. et al. A randomized double-blind phase III study of nimorazole as a hypoxic radiosensitizer of primary radiotherapy in supraglottic larynx and pharynx carcinoma. Results of the Danish Head and Neck Cancer Study (DAHANCA) Protocol 5-85. Radiother Oncol 46, 135-46 (1998).
106. Overgaard, J., Eriksen, J. G., Nordsmark, M., Alsner, J. & Horsman, M. R. Plasma osteopontin, hypoxia, and response to the hypoxia sensitiser nimorazole in radiotherapy of head and neck cancer: results from the DAHANCA 5 randomised double-blind placebo-controlled trial. Lancet Oncol 6, 757-64 (2005).
107. Rischin, D. et al. Prognostic significance of [18F]-misonidazole positron emission tomography-detected tumor hypoxia in patients with advanced head and neck cancer randomly assigned to chemoradiation with or without tirapazamine: a substudy of Trans-Tasman Radiation Oncology Group Study 98.02. J Clin Oncol 24, 2098-104 (2006).
108. Jain R K. Normalization of tumor vasculature: an emerging concept in antiangiogenic therapy. Science. 2005; 307:58-62.
109. Willett C G, Boucher Y, di Tomaso E, et al. Direct evidence that the VEGF-specific antibody bevacizumab has antivascular effects in human rectal cancer. Nat Med. 2004; 10:145-147.
110. Rischin D, Peters L, Fisher R, et al. Tirapazamine, Cisplatin, and Radiation versus Fluorouracil, Cisplatin, and Radiation in patients with locally advanced head and neck cancer: a randomized phase II trial of the Trans-Tasman Radiation Oncology Group (TROG 98.02). J Clin Oncol. 2005; 23:79-87.
111. Le Q T, Taira A, Budenz S, et al. Mature results from a randomized Phase II trial of cisplatin plus 5-fluorouracil and radiotherapy with or without tirapazamine in patients with resectable Stage 1V head and neck squamous cell carcinomas. Cancer. 2006; 106:1940-1949.
112. O'Rourke J F, Dachs G U, Gleadle J M, Maxwell P H, Pugh C W, Stratford I J, et al. Hypoxia response elements. Oncol Res 1997; 9:327-32.
113. Zhong H, De Marzo A M, Laughner E, Lim M, Hilton D A, Zagzag D, et al. Overexpression of hypoxia-inducible factor 1{{alpha}} in common human cancers and their metastases. Cancer Res 1999; 59:5830-5.
114. Talks K L, Turley H, Gatter K C, Maxwell P H, Pugh C W, Ratcliffe P J, et al. The expression and distribution of the hypoxia-inducible factors HIF-1{alpha} and HIF-2{alpha} in normal human tissues, cancers, and tumor-associated macrophages. Am J Pathol 2000; 157:411-21.[
115. Chadderton N, Cowen R L, Sheppard F C, Robinson S, Greco O, Scott S D, et al. Dual responsive promoters to target therapeutic gene expression to radiation-resistant hypoxic tumor cells. Int J Radiat Oncol Biol Phys 2005; 62:213-22.[
116. Dachs G U, Patterson A V, Firth J D, Ratcliffe P J, Townsend K M, Stratford I J, et al. Targeting gene expression to hypoxic tumor cells. Nat Med 1997; 3:515-20
117. Patterson A V, Williams K J, Cowen R L, Jaffar M, Telfer B A, Saunders M, et al. Oxygen-sensitive enzyme-prodrug gene therapy for the eradication of radiation-resistant solid tumours. Gene Ther 2002; 9:946-54.
118. Matzow T, Cowen R L, Williams K J, Telfer B A, Flint P J, Southgate T D, et al. Hypoxia-targeted over-expression of carboxylesterase as a means of increasing tumour sensitivity to irinotecan (CPT-11). J Gene Med 2007; 9:244-52.[
119. Shibata T, Akiyama N, Noda M, Sasai K, Hiraoka M. Enhancement of gene expression under hypoxic conditions using fragments of the human vascular endothelial growth factor and the erythropoietin genes. Int J Radiat Oncol Biol Phys 1998; 42:913-6.[
120. Koshikawa N, Takenaga K, Tagawa M, Sakiyama S. Therapeutic efficacy of the suicide gene driven by the promoter of vascular endothelial growth factor gene against hypoxic tumor cells. Cancer Res 2000; 60:2936-41.
121. Ruan H, Su H, Hu L, Lamborn K R, Kan Y W, Deen D F. A hypoxia-regulated adeno-associated virus vector for cancer-specific gene therapy. Neoplasia 2001; 3:255-63.
122. Wang D, Ruan H, Hu L, Lamborn K R, Kong E L, Rehemtulla A, et al. Development of a hypoxia-inducible cytosine deaminase expression vector for gene-directed prodrug cancer therapy. Cancer Gene Ther 2005; 12:276-83.
123. Cowen R L, Williams K J, Chinje E C, Jaffar M, Sheppard F C, Telfer B A, et al. Hypoxia targeted gene therapy to increase the efficacy of tirapazamine as an adjuvant to radiotherapy: reversing tumor radioresistance and effecting cure. Cancer Res 2004; 64:1396-402.
124. Shibata T, Giaccia A J, Brown J M. Hypoxia-inducible regulation of a prodrug-activating enzyme for tumor-specific gene therapy. Neoplasia 2002; 4:40-8.
125. Ozawa T, Hu J L, Hu L J, Kong E L, Bollen A W, Lamborn K R, et al. Functionality of hypoxia-induced BAX expression in a human glioblastoma xenograft model. Cancer Gene Ther 2005; 12:449-551
126. Salloum R M, Saunders M P, Mauceri H J, Hanna N N, Gorski D H, Posner M C, et al. Dual induction of the Epo-Egr-TNF-alpha-plasmid in hypoxic human colon adenocarcinoma produces tumor growth delay. Am Surg 2003; 69:24-7.
127. Post D E, Sandberg E M, Kyle M M, Devi N S, Brat D J, Xu Z, et al. Targeted cancer gene therapy using a hypoxia inducible factor dependent oncolytic adenovirus armed with interleukin-4. Cancer Res 2007; 67:6872-81.
128. Post D E, Van Meir E G. A novel hypoxia-inducible factor (HIF) activated oncolytic adenovirus for cancer therapy. Oncogene 2003; 22:2065-72.
129. McKeown S R, Cowen R L, Williams K J. Bioreductive drugs: from concept to clinic. Clin Oncol (R Coll Radiol) 2007; 19:427-42.
130. Stratford I J, Williams K J, Cowen R L, Jaffar M. Combining bioreductive drugs and radiation for the treatment of solid tumors. [Review] [83 refs]. Semin Radiat Oncol 2003; 13:42-52.

### REFERENCES TO EXAMPES

Beer D G, Kardia S L, Huang C C, Giordano T J, Levin A M, Misek D E, Lin L, Chen G, Gharib T G, Thomas D G, Lizyness M L, Kuick R, Hayasaka S, Taylor J M, Iannettoni M D, Orringer M B, Hanash S (2002) Gene-expression profiles predict survival of patients with lung adenocarcinoma. Nat Med 8: 816-24
Butte A J, Kohane I S (2003) Relevance Networks: A first step towards finding genetic regulatory networks within microarray data. In The Analysis of Gene Expression Data, Parmigiani G, Gar-rett E S, Irizarry R A, Zeger S (eds). New York: Springer-Verla
Carroll J S, Meyer C A, Song J, Li W, Geistlinger T R, Eeckhoute J, Brodsky A S, Keeton E K, Fertuck K C, Hall G F, Wang Q, Bekiranov S, Sementchenko V, Fox E A, Silver P A, Gingeras T R, Liu X S, Brown M (2006) Genome-wide analysis of estrogen receptor binding sites. Nat Genet 38: 1289-97
Chi J T, Wang Z, Nuyten D S, Rodriguez E H, Schaner M E, Salim A, Wang Y, Kristensen G B, Helland A, Børresen-Dale A L, Giaccia A, Longaker M T, Hastie T, Yang G P, van de Vijver M J, Brown P O (2006) Gene expression programs in response to hypoxia: cell type specificity and prognostic significance in human cancers. PLoS Med 3: e47
Choi P, Chen C (2005) Genetic expression profiles and biologic pathway alterations in head and neck squamous cell carcinoma. Cancer 104: 1113-28
Chung C H, Parker J S, Karaca G, Wu J, Funkhouser W K, Moore D, Butterfoss D, Xiang D, Zanation A, Yin X, Shockley W W, Weissler M C, Dressler L G, Shores C G, Yarbrough W G, Perou C M (2004) Molecular classification of head and neck squamous cell carcinomas using patterns of gene expression. Cancer Cell 5: 489-500
Cromer A, Carles A, Millon R, Ganguli G, Channel F, Lemaire F, Young J, Dembélé D, Thibault C, Muller D, Poch O, Abecassis J, Wasylyk B (2004) Identification of genes associated with tumorigenesis and metastatic potential of hypopharyngeal cancer by microarray analysis. Oncogene 23: 2484-98
Desmedt C, Haibe-Kains B, Wirapati P, Buyse M, Larsimont D, Bontempi G, Delorenzi M, Piccart M, Sotiriou C (2008) Biological processes associated with breast cancer clinical outcome depend on the molecular subtypes. Clin Cancer Res 14: 5158-65
Elvidge G P, Glenny L, Appelhoff R J, Ratcliffe P J, Ragoussis J, Gleadle J M (2006) Concordant regulation of gene expression by hypoxia and 2-oxoglutarate-dependent dioxygenase inhibition: the role of HIF-1alpha, HIF-2alpha, and other pathways. J Biol Chem 281: 15215-26
Fox S B, Generali D G, Harris A L (2007) Breast tumour angiogenesis. Breast Cancer Res 9: 216
Hahn M W, Kern A D (2005) Comparative genomics of centrality and essentiality in three eukaryotic protein-interaction networks. Mol Biol Evol 22: 803-6
Harris A L (2002) Hypoxia—a key regulatory factor in tumour growth. Nat Rev Cancer 2: 38-47
Hastie R, Tibshirani J, Friedman H (2001) The Elements of Statistical Learning: Data Mining, Inference, and Prediction. New York: Springer-Verla
Loi S, Haibe-Kains B, Desmedt C, Wirapati P, Lallemand F, Tutt A M, Gillet C, Ellis P, Ryder K, Reid J F, Daidone M G, Pierotti M A, Berns E M, Jansen M P, Foekens J A, Delorenzi M, Bontempi G, Piccart M J, Sotiriou C (2008) Predicting prognosis using molecular profiling in estrogen receptor-positive breast cancer treated with tamoxifen. BMC Genomics 9: 239
Miller L D, Smeds J, George J, Vega V B, Vergara L, Ploner A, Pawitan Y, Hall P, Klaar S, Liu E T, Bergh J (2005) An expression signature for p53 status in human breast cancer predicts mutation status, transcriptional effects, and patient survival. Proc Natl Acad Sci USA 102: 13550-5
Nordsmark M, Bentzen S M, Rudat V, Brizel D, Lartigau E, Stadler P, Becker A, Adam M, Molls M, Dunst J, Terris D J, Overgaard J (2005) Prognostic value of tumor oxygenation in 397 head and neck tumors after primary radiation therapy. An international multi-center study. Radiother Oncol 77: 18-24
Oliver R J, Woodwards R T, Sloan P, Thakker N S, Stratford I J, Airley R E (2004) Prognostic value of facilitative glucose transporter Glut-1 in oral squamous cell carcinomas treated by surgical resection; results of EORTC Translational Research Fund studies. Eur J Cancer 40: 503-7
Pyeon D, Newton M A, Lambert P F, den Boon J A, Sengupta S, Marsit C J, Woodworth C D, Connor J P, Haugen T H, Smith E M, Kelsey K T, Turek L P, Ahlquist P (2007) Fundamental differences in cell cycle deregulation in human papillomavirus-positive and human papillomavirus-negative head/neck and cervical cancers. Cancer Res 67: 4605-19
Raponi M, Zhang Y, Yu J, Chen G, Lee G, Taylor J M, Macdonald J, Thomas D, Moskaluk C, Wang Y, Beer DG (2006) Gene expression signatures for predicting prognosis of squamous cell and adenocarcinomas of the lung. Cancer Res 66: 7466-72
Subramanian A, Tamayo P, Mootha V K, Mukherjee S, Ebert B L, Gillette M A, Paulovich A, Pomeroy S L, Golub T R, Lander E S, Mesirov J P (2005) Gene set enrichment analysis: a knowledge-based approach for interpreting genome-wide expression profiles. Proc Natl Acad Sci USA 102: 15545-50
van de Vijver M J, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, Bernards R (2002) A gene-expression signature as a predictor of survival in breast cancer. N Engl J Med 347: 1999-2009
Wilson C L, Miller C J (2005) Simpleaffy: a BioConductor package for Affymetrix Quality Control and data analysis. Bioinformatics 21: 3683-5
Winter S C, Buffa F M, Silva P, Miller C, Valentine H R, Turley H, Shah K A, Cox G J, Corbridge R J, Horner J J, Musgrove B, Slevin N, Sloan P, Price P, West C M, Harris A L (2007) Relation of a hypoxia metagene derived from head and neck cancer to prognosis of multiple cancers. Cancer Res 67: 3441-9
Wolfe C J, Kohane I S, Butte A J (2005) Systematic survey reveals general applicability of “guilt-by-association” within gene coexpression networks. BMC Bioinformatics 6: 227
Buffa F M, Harris A L, West C M and Miller C J (2010) Large meta-analysis of multiple cancers reveals a common compact and highly pronostic hypoxia metagene. British Journal of Cancer 102: 428-435.

1. A method for assessing a hypoxia phenotype of a tumour of a subject, comprising:
determining the gene expression of between 3 and 50 hypoxia-related genes of a sample obtained from said tumour of the subject, thereby obtaining a sample expression profile of said hypoxia-related genes; and
comparing the sample gene expression profile with a reference expression profile of said hypoxia-related genes,
wherein said hypoxia-related genes comprise at least SLC2A1, VEGFA and PGAM1.

2. The method according to claim 1, wherein said hypoxia-related genes comprise, in addition to SLC2A1, VEGFA and PGAM1, at least 2, 3, 4, 5, 10, 15 or at least 20 genes selected from the group consisting of: PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1.

3. The method according to claim 1, wherein said hypoxia-related genes comprise, in addition to SLC2A1, VEGFA and PGAM1, at least 70% of the genes selected from the group consisting of: PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, KCTD11, ANGPTL4 and FOSL1, and optionally KRT17, PPM1J and/or HIG2.

4. The method according to claim 1, wherein said hypoxia-related genes consist of the 25-gene set: SLC2A1, VEGFA, PGAM1, PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein PPM1J may optionally be replaced by HIG2.

5. The method according to claim 1, wherein said hypoxia-related genes consist of the 26-gene set: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein PPM1J may optionally be replaced by HIG2.

6. The method according to claim 1, wherein the method further comprises determining the gene expression of at least 1, 2, 3, 4, 5, or more control genes of said sample.

7. The method according to claim 1, wherein the tumour is selected from: a tumour of the head and/or neck, including a head and neck squamous cell carcinoma (HNSCC); breast cancer tumour; a lung cancer tumour; a cervical cancer tumour; and a bladder cancer tumour.

8. The method according to claim 1, wherein determining the expression of said hypoxia-related genes comprises quantitative PCR (qPCR) and/or use of a DNA microarray.

9. The method according to claim 8, wherein the method comprises, prior to carrying out qPCR, extracting RNA from a fresh or processed tissue sample that has been obtained from said tumour and reverse transcribing said RNA.

10. The method according to claim 1, wherein comparing the sample gene expression profile with the reference expression profile comprises:
(a) quantitatively comparing the gene expression level of each of said hypoxia-related genes of said tumour with a reference expression level for the respective hypoxia-related gene from a set of tumours of known hypoxia phenotype; and/or
(b) quantitatively scoring the gene expression level of each of said hypoxia-related genes of said tumour, thereby deriving an overall sample score for the sample gene expression profile, and comparing the overall sample score with an overall reference score derived from the expression level of each of said hypoxia-related genes from a set of tumours of known hypoxia phenotype.

11. The method according to claim 10, wherein the expression level of each of said hypoxia-related genes is normalised to the expression of one or more control genes.

12. The method according to claim 1, wherein said tumour is classified as hypoxic.

13. A method for prognosing a subject having a tumour, comprising assessing the hypoxia phenotype of said tumour by the method of claim 1, wherein a greater degree of similarity between the sample expression profile and the reference expression profile indicates a less favourable prognosis for the subject.

14. A method according to claim 13, wherein the method is for determining overall survival time, metastases-free survival time, recurrence-free survival time and/or disease-specific survival time, of the subject.

15. A method according to claim 13, wherein the method comprises assessing the hypoxia phenotype of a tumour from each of a plurality of subjects, and stratifying said plurality of subjects according to the severity of their prognosis.

16. A method for predicting or assessing response to hypoxia modification therapy or hypoxia targeted therapy in a subject having a tumour, comprising assessing the hypoxia phenotype of said tumour by the method of claim 1, wherein a greater degree of similarity between the sample expression profile and the reference expression profile indicates an increased likelihood that the subject will benefit from hypoxia modification therapy.

17. A method according to claim 1, wherein:
said hypoxia-related genes are selected from the human hypoxia-related genes having the nucleotide sequences set forth in Table 10.

18. A set of at least one of probes and primers for use in a method according to claim 1, comprising: a plurality of oligonucleotides capable of hybridising to between 3 and 50 hypoxia-related genes, wherein said hypoxia-related genes comprise at least SLC2A1, VEGFA and PGAM1.

19. The set according to claim 18, wherein said hypoxia-related genes comprise, in addition to SLC2A1, VEGFA and PGAM1, at least 2, 3, 4, 5, 10, 15 or at least 20 genes selected from the group consisting of: PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1.

20. The set according to claim 18, wherein said hypoxia-related genes comprise, in addition to SLC2A1, VEGFA and PGAM1, at least 70% of the genes selected from the group consisting of: PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, KCTD11, ANGPTL4 and FOSL1, and optionally KRT17, PPM1J and/or HIG2.

21. The set according to claim 18, wherein said hypoxia-related genes consist of: SLC2A1, VEGFA, PGAM1, PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein said PPM1J may optionally be replaced by HIG2.

22. The set according to claim 18, wherein said hypoxia-related genes consist of: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1.

23. The set according to claim 1, wherein further comprising probes and/or primers capable of hybridising to 1, 2, 3, 4, 5, or more control genes.

24. The set according to claim 18, wherein the oligonucleotide probes and/or primers are provided in an array on a solid support or are coupled to a plurality of labelled beads.

25. A TaqMan® qPCR array for use in a method according to claim 1, comprising a micro-fluidic card pre-loaded with primers for amplification of:
between 3 and 50 hypoxia-related genes, wherein said hypoxia-related genes comprise at least SLC2A1, VEGFA and PGAM1; and
optionally, one or more control genes that are not hypoxia-related.

26. The TaqMan® qPCR array of claim 25, wherein said micro-fluidic card is pre-loaded with primers for amplification of, in addition to SLC2A1, VEGFA and PGAM1, at least 70% of the genes selected from: PGK1, SLC16A1, ENO1, BNC1, LDHA, TPIL CA9, SDC1, DCBLD1, ALDOA, FAM83B, GNAI1, CDKN3, ANLN, C20orf20, MRPS17, COL4A6, P4HA1, KCTD11, ANGPTL4 and FOSL1, and optionally KRT17, PPM1J and/or HIG2; and
optionally, one or more control genes that are not hypoxia-related.

27. The TaqMan® qPCR array of claim 25, wherein said micro-fluidic card is pre-loaded with primers for amplification of:
the 25-gene hypoxia signature set consisting of: SLC2A1, VEGFA, PGAM1, PGK1, SLC16A1, ENO1, BNC1, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1, wherein said PPM1J may optionally be replaced by HIG2; and
optionally, one or more control genes that are not hypoxia-related.

28. The TaqMan® qPCR array of claim 25, wherein said micro-fluidic card is pre-loaded with primers for amplification of:
the 26-gene hypoxia signature set consisting of: SLC2A1, VEGFA, PGAM1, PGK1, SLC16A1, ENO1, BNC1, KRT17, LDHA, TPI1, CA9, SDC1, DCBLD1, ALDOA, FAM83B, GNAI1, CDKN3, ANLN, C20orf20, MRPS17, COL4A6, P4HA1, PPM1J, KCTD11, ANGPTL4 and FOSL1; and
optionally, one or more control genes that are not hypoxia-related.

29. A kit for use in a method according to claim 1, comprising:
the set according to claim 18 or the TaqMan® qPCR array of claim 25; and
instructions, controls and/or reagents for performing a method according to claim 1.

30. A method according to claim 11, wherein said control genes are selected from the human control genes having the nucleotide sequences set forth in Table 10.


SEQUENCE LISTING (57 sequences)

SEQ ID NO: 1 - DNA, 3687 bp, Homo sapiens
tccaccattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggccactcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctctggcaagaggcaagaggtagcaacagcgagcgtgccggtcgctagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctccccactctaggggtcaggctccattaggatttgccccttcccatctcttcctacccaaccactcaaattaatctttctttacctgagaccagttgggagcactggagtgcagggaggagaggggaagggccagtctgggctgccgggttctagtctcctttgcactgagggccacactattaccatgagaagagggcctgtgggagcctgcaaactcactgctcaagaagacatggagactcctgccctgttgtgtatagatgcaagatatttatatatatttttggttgtcaatattaaatacagacactaagttatagtatatctggacaagccaacttgtaaatacaccacctcactcctgttacttacctaaacagatataaatggctggtttttagaaacatggttttgaaatgcttgtggattgagggtaggaggtttggatgggagtgagacagaagtaagtggggttgcaaccactgcaacggcttagacttcgactcaggatccagtcccttacacgtacctctcatcagtgtcctcttgctcaaaaatctgtttgatccctgttacccagagaatatatacattctttatcttgacattcaaggcatttctatcacatatttgatagttggtgttcaaaaaaacactagttttgtgccagccgtgatgctcaggcttgaaatgcattattttgaatgtgaagtaaatactgtacctttattggacaggctcaaagaggttatgtgcctgaagtcgcacagtgaataagctaaaacacctgcttttaacaatggtaccatacaaccactactccattaactccacccacctcctgcacccctccccacacacacaaaatgaaccacgttctttgtatgggcccaatgagctgtcaagctgccctgtgttcatttcatttggaattgccccctctggttcctctgtatactactgcttcatctctaaagacagctcatcctcctccttcacccctgaatttccagagcacttcatctgctccttcatcacaagtccagttttctgccactagtctgaatttcatgagaagatgccgatttggttcctgtgggtcctcagcactattcagtacagtgcttgatgcacagcaggcactcagaaaatactggaggaaataaaacaccaaagatatttgtcaaaaaaaaaaaaaaaaa

SEQ ID NO: 2 - DNA, 3626 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 3 - DNA, 3677 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 4 - DNA, 3608 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccgtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 5 - DNA, 3554 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 6 - DNA, 3554 bp, Homo sapiens; identical to SEQ ID NO: 5

SEQ ID NO: 7 - DNA, 3608 bp, Homo sapiens; identical to SEQ ID NO: 4

SEQ ID NO: 8 - DNA, 3626 bp, Homo sapiens; identical to SEQ ID NO: 2

SEQ ID NO: 9 - DNA, 3677 bp, Homo sapiens; identical to SEQ ID NO: 3

SEQ ID NO: 10 - DNA, 1762 bp, Homo sapiens
gctaatcccagtcggtgccgcatccccagcccgccgccatggccgcctacaaactggtgctgatccggcacggcgagagcgcatggaacctggagaaccgcttcagcggctggtacgacgccgacctgagcccggcgggccacgaggaggcgaagcgcggcgggcaggcgctacgagatgctggctatgagtttgacatctgcttcacctcagtgcagaagagagcgatccggaccctctggacagtgctagatgccattgatcagatgtggctgccagtggtgaggacttggcgcctcaatgagcggcactatgggggtctaaccggtctcaataaagcagaaactgctgcaaagcatggtgaggcccaggtgaagatctggaggcgctcctatgatgtcccaccacctccgatggagcccgaccatcctttctacagcaacatcagtaaggatcgcaggtatgcagacctcacagaagatcagctaccctcctgtgagagtctgaaggatactattgccagagctctgcccttctggaatgaagaaatagttccccagatcaaggaggggaaacgtgtactgattgcagcccatggcaacagcctccggggcattgtcaagcatctggagggtctctctgaagaggctatcatggagctgaacctgccgactggtattcccattgtctatgaattggacaagaacttgaagcctatcaagcccatgcagtttctgggggatgaagagacggtgcgcaaagccatggaagctgtggctgcccagggcaaggccaagaagtgaaggccggcggggaggatactgtccccaggagcaccctccctgcccgtcttgtccctctgcccctcccacctgcacatgtcacactgaccacatctgtagacatcttgagttgtagctgcagacggggaccagtggctcccattttcattttagccattttgtcgcctgcacccactcccttcatacaatctagtcagaatagcagttctagagcacaggttctcagtctaagctatggaaaagctccccttatccaacagagtttaaaagtagtgacttgggtttttgcgagtgctttgtttactaaggactttggggaggaaccatgctaagccatgaccagtgaggagaagcaacagagcctgtctgtccccatgagcggagtctgtcctctgctcttctgcagtcaggtcactgcctactgcctgggggctctagtcattccagtggaagacgaatgtaacctgcgtggtgatgtgacaactgtttcctccctgaccccagaggatctggctctaggttgggatcaatcctgaatttcgttatgtgttaatttacttttattaaaaaagtatagtatatataatacaaaacaataacccttctggggtttcttgtggcggttgaaatagtcccacatgtggtcatcagaaaataagccattcctcataccaatataggatcagctccttgacctctgaggggcaggagtgcttcctggtgtgtgtattagaatcccttcctgccttgtttcatggcagtgaaatgcctcttggtcctgtccaagtgtatctttcactgatttctgaatcatgttctagttgcttgaccctgccacatgggtccagtgttcatctgagcataactgtactaaatcctttttccatatcagtataataaaggagtgatgtgcaataaaaaaaaaaaaaaaaaaaaaaaaaaaaaaaaaaaaaaaaaaa

SEQ ID NO: 11 - DNA, 2439 bp, Homo sapiens
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 12 - DNA, 3927 bp, Homo sapiens
agagggcgcgcgcggctgaaagcgtgtggaggcgcgggctgcagttcggatgtctgtgtggcggggagggggcggcggccgggagagacgactccgccccctgcgcgcatgctccggccccggcgggttataaggcagcctcgctggcccggccagacaaagtggtgagctgcgacgtgactggctagctgcgtgggtactggaacaagcaaacgaggcagcgagcgaaggacgggagccggaccctgggccccgtggaactccagcctgcgccaccacgtcacgcacacgctcggcgctgcgatccgcgcatataacgatatttggatttgacctgcattttggaatttatctacacttaaaatgccaccagcagttggaggtccagttggatacacccccccagatggaggctggggctgggcagtggtaattggagctttcatttccatcggcttctcttatgcatttcccaaatcaattactgtcttcttcaaagagattgaaggtatattccatgccaccaccagcgaagtgtcatggatatcctccataatgttggctgtcatgtatggtggaggtcctatcagcagtatcctggtgaataaatatggaagtcgtatagtcatgattgttggtggctgcttgtcaggctgtggcttgattgcagcttctttctgtaacaccgtacagcaactatacgtctgtattggagtcattggaggtcttgggcttgccttcaacttgaatccagctctgaccatgattggcaagtatttctacaagaggcgaccattggccaacggactggccatggcaggcagccctgtgttcctctgtactctggcccccctcaatcaggttttcttcggtatctttggatggagaggaagctttctaattcttgggggcttgctactaaactgctgtgttgctggagccctcatgcgaccaatcgggcccaagccaaccaaggcagggaaagataagtctaaagcatcccttgagaaagctggaaaatctggtgtgaaaaaagatctgcatgatgcaaatacagatcttattggaagacaccctaaacaagagaaacgatcagtcttccaaacaattaatcagttcctggacttaaccctattcacccacagaggctttttgctatacctctctggaaatgtgatcatgttttttggactctttgcacctttggtgtttcttagtagttatgggaagagtcagcattattctagtgagaagtctgccttccttctttccattctggcttttgttgacatggtagcccgaccatctatgggacttgtagccaacacaaagccaataagacctcgaattcagtatttctttgcggcttccgttgttgcaaatggagtgtgtcatatgctagcacctttatccactacctatgttggattctgtgtctatgcgggattctttggatttgccttcgggtggctcagctccgtattgtttgaaacattgatggaccttgttggaccccagaggttctccagcgctgtgggattggtgaccattgtggaatgctgtcctgtcctcctggggccaccacttttaggtcggctcaatgacatgtatggagactacaaatacacatactgggcatgtggcgtcgtcctaattatttcaggtatctatctcttcattggcatgggcatcaattatcgacttttggcaaaagaacagaaagcaaacgagcagaaaaaggaaagtaaagaggaagagaccagtatagatgttgctgggaagccaaatgaagttaccaaagcagcagaatctccggaccagaaagacacagatggagggcccaaggaggaggaaagtccagtctgaatccatggggctgaagggtaaattgagcagttcatgacccaggatatctgaaaatattctactggcctgtaatctaccagtggtgctcaatgcaaatagtagacatttgtgtggaaatcataccagttgttcattgatgggatttttgtttgactccttaccaatagcctgaatttgaggagggaatgattggtagcaaaggatgggggaaagaagtaggttctgttttgttttgttttaatcttagcttttaatagtgtcataaagattataatatgtgccttaagttttagtctttagaactctagagagccttaacttcttaaaccatttttgctgaattcatctatttcgagtgttgtgttaaaaggaaaaataacaactaacttgtttgaggcaaatctaaaatttaaaattaatcttgcttcattgttacatgtaatatatttcagacattttcactggaagatttatgaacagaaatattggttgaaagttagagattttacaaaatgctgacaaaaatattttcctagcatcagtagatttctggcatatgtttctgctagctatatatttaggaaattcaaagcataaaactttggcaacatcttggctgttctagacacagtgtacttgtcaacccctctcaggtaccttttcttgggatgcttattagaagccaagtaaagtgcttaaggtttgttttcattaaattagctatttctgctcccctgttcaaagatgcattttgagtgtttatagatcactgccctttttgaaatcacctggtattatttttcttactggaaaagttagtattaaaatctacagaactacatatttgtgcctccttggtaaatacaacacatctaattaaatgtagacagatatttcaaacatcagctgaattcacttaagtttttccaaaacctcagttaaactgtgaagctattggaatttttttttcctggaatttttcccctttgattcacagtggtcccatttatatctgcttctagcttagtgctatgtgtgagatatgtgtgtgtttggtgtttttgtttttttgtttttttttttttaaggtttgcaaattaaaaagggccagaaaaatttggcaccaggcaaacgaataaagataggattgggaaagaagttgctaagtgtgcttagttttaataagtaattccttctcttttttcagagaaggccttacagaaaattgttgtgcttagaattgctggatgcatttttaccctccacacaaacctaaaaattttgtgacccctttcacttacctgaaaagtagagaaatggattcagtataaggataaggagggaaggtggaccagaatgaaaactgtaaatatttttttaacctaatatcacttaaatcgaggcagaaagatacagacattcaatgaattatattcaatgcatttaaaataccactgtaattgacagagtaaaagtatagatacaaaaccttgtgtaagaggctgacttttccaaataaacattttttaagaaaacatttcttctcccaaatgtctattttcttgaggaaaatattgctgtgtcttcattttcattaccaggtttcattttgggccttgctaaattgattgaattaaatcctccagcttttgaaccttgatatttgtgtatatgatttattttcatttgaatttctcctttcctcttctttgctgtaaggcaaggaggaggggaattttaaaaccatcttatttgaactgagagcatccagagcagttaaccttaaggaaacaatgaaaaactccctttgtatgcctgggcatcatggcagatagaggaagagtgttagaggagaaaactgctgctgagagtattggcaggcttggcctcagtttggactctgtaattttctttggacccaagtctgtaacctctgcgtacttcttctctcttaccttctataaaaatgaggattactgttggtgagggaataaggaatgtaagtaaaggaaatctgaaaaaataaaagtaaagcaagtataaaaaaaaaaaaaaaaaaaa

SEQ ID NO: 13 - DNA, 4390 bp, Homo sapiens
gctcggcgctgcgatccgcgcatataacggttagttgggtaacagccggcggcacgcggcgcggaccccacggtgccctcgctgccctggtggggtcggaggggacctccggggttgggagactttgtctccggcggagggaggcggcccagcagagggcatcgtggtcacaggcagccgcgtggcctcggactgcagtgctggtgaaggagaccttgaggcgctggggtcagcgcctctcttagccgagccgcgggccccgtgctaagatgccggtcccaggcctggccgaggagttgggtcgtggtgcccggtgacggtggggaagtcccttcccccctaagtcttcaataggctcctcgagagatgctttgatctggaaaagggataacatgaggctgctgcatgtggtacctccagactctcctggccgcttctgccccaagggaagggactcggggcagaagttatttatgattcgtgacattgtccgccccaaccttcttggcccgccgatccttgccccacctggtgacttgcgagagtggtgcggagagccgcattccccacagccaaggcgtgactgcagggttcgagtagactttgcggaggggacggggcagcccgcagactcctgggagtggtgctgacgggggctctttgtcattcaacctgtggctgccagcgtccgccgcagcgcccttctactcatggacttaaggcggccctgttgagagaaatctggaggatagcgttacacttggtccgatgcaagttttttgttccaaatatttggatttgacctgcattttggaatttatctacacttaaaatgccaccagcagttggaggtccagttggatacacccccccagatggaggctggggctgggcagtggtaattggagctttcatttccatcggcttctcttatgcatttcccaaatcaattactgtcttcttcaaagagattgaaggtatattccatgccaccaccagcgaagtgtcatggatatcctccataatgttggctgtcatgtatggtggaggtcctatcagcagtatcctggtgaataaatatggaagtcgtatagtcatgattgttggtggctgcttgtcaggctgtggcttgattgcagcttctttctgtaacaccgtacagcaactatacgtctgtattggagtcattggaggtcttgggcttgccttcaacttgaatccagctctgaccatgattggcaagtatttctacaagaggcgaccattggccaacggactggccatggcaggcagccctgtgttcctctgtactctggcccccctcaatcaggttttcttcggtatctttggatggagaggaagctttctaattcttgggggcttgctactaaactgctgtgttgctggagccctcatgcgaccaatcgggcccaagccaaccaaggcagggaaagataagtctaaagcatcccttgagaaagctggaaaatctggtgtgaaaaaagatctgcatgatgcaaatacagatcttattggaagacaccctaaacaagagaaacgatcagtcttccaaacaattaatcagttcctggacttaaccctattcacccacagaggctttttgctatacctctctggaaatgtgatcatgttttttggactctttgcacctttggtgtttcttagtagttatgggaagagtcagcattattctagtgagaagtctgccttccttctttccattctggcttttgttgacatggtagcccgaccatctatgggacttgtagccaacacaaagccaataagacctcgaattcagtatttctttgcggcttccgttgttgcaaatggagtgtgtcatatgctagcacctttatccactacctatgttggattctgtgtctatgcgggattctttggatttgccttcgggtggctcagctccgtattgtttgaaacattgatggaccttgttggaccccagaggttctccagcgctgtgggattggtgaccattgtggaatgctgtcctgtcctcctggggccaccacttttaggtcggctcaatgacatgtatggagactacaaatacacatactgggcatgtggcgtcgtcctaattatttcaggtatctatctcttcattggcatgggcatcaattatcgacttttggcaaaagaacagaaagcaaacgagcagaaaaaggaaagtaaagaggaagagaccagtatagatgttgctgggaagccaaatgaagttaccaaagcagcagaatctccggaccagaaagacacagatggagggcccaaggaggaggaaagtccagtctgaatccatggggctgaagggtaaattgagcagttcatgacccaggatatctgaaaatattctactggcctgtaatctaccagtggtgctcaatgcaaatagtagacatttgtgtggaaatcataccagttgttcattgatgggatttttgtttgactccttaccaatagcctgaatttgaggagggaatgattggtagcaaaggatgggggaaagaagtaggttctgttttgttttgttttaatcttagcttttaatagtgtcataaagattataatatgtgccttaagttttagtctttagaactctagagagccttaacttcttaaaccatttttgctgaattcatctatttcgagtgttgtgttaaaaggaaaaataacaactaacttgtttgaggcaaatctaaaatttaaaattaatcttgcttcattgttacatgtaatatatttcagacattttcactggaagatttatgaacagaaatattggttgaaagttagagattttacaaaatgctgacaaaaatattttcctagcatcagtagatttctggcatatgtttctgctagctatatatttaggaaattcaaagcataaaactttggcaacatcttggctgttctagacacagtgtacttgtcaacccctctcaggtaccttttcttgggatgcttattagaagccaagtaaagtgcttaaggtttgttttcattaaattagctatttctgctcccctgttcaaagatgcattttgagtgtttatagatcactgccctttttgaaatcacctggtattatttttcttactggaaaagttagtattaaaatctacagaactacatatttgtgcctccttggtaaatacaacacatctaattaaatgtagacagatatttcaaacatcagctgaattcacttaagtttttccaaaacctcagttaaactgtgaagctattggaatttttttttcctggaatttttcccctttgattcacagtggtcccatttatatctgcttctagcttagtgctatgtgtgagatatgtgtgtgtttggtgtttttgtttttttgtttttttttttttaaggtttgcaaattaaaaagggccagaaaaatttggcaccaggcaaacgaataaagataggattgggaaagaagttgctaagtgtgcttagttttaataagtaattccttctcttttttcagagaaggccttacagaaaattgttgtgcttagaattgctggatgcatttttaccctccacacaaacctaaaaattttgtgacccctttcacttacctgaaaagtagagaaatggattcagtataaggataaggagggaaggtggaccagaatgaaaactgtaaatatttttttaacctaatatcacttaaatcgaggcagaaagatacagacattcaatgaattatattcaatgcatttaaaataccactgtaattgacagagtaaaagtatagatacaaaaccttgtgtaagaggctgacttttccaaataaacattttttaagaaaacatttcttctcccaaatgtctattttcttgaggaaaatattgctgtgtcttcattttcattaccaggtttcattttgggccttgctaaattgattgaattaaatcctccagcttttgaaccttgatatttgtgtatatgatttattttcatttgaatttctcctttcctcttctttgctgtaaggcaaggaggaggggaattttaaaaccatcttatttgaactgagagcatccagagcagttaaccttaaggaaacaatgaaaaactccctttgtatgcctgggcatcatggcagatagaggaagagtgttagaggagaaaactgctgctgagagtattggcaggcttggcctcagtttggactctgtaattttctttggacccaagtctgtaacctctgcgtacttcttctctcttaccttctataaaaatgaggattactgttggtgagggaataaggaatgtaagtaaaggaaatctgaaaaaataaaagtaaagcaagtataaaaaaaaaaaaaa

SEQ ID NO: 14 - DNA, 1812 bp, Homo sapiens
tagctaggcaggaagtcggcgcgggcggcgcggacagtatctgtgggtacccggagcacggagatctcgccggctttacgttcacctcggtgtctgcagcaccctccgcttcctctcctaggcgacgagacccagtggctagaagttcaccatgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagcccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagctttgcagtcgtgtaattggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgag

SEQ ID NO: 15 - DNA, 4627 bp, Homo sapiens
gcggcgggggcggccatcgtgctgcgcagcctgggcgcttggggagccgcccacttcgccgggtcgcgccccgacggccggagcgtggatgcggcggcgcccgccgagccggggcggacgcggggcggcccgggcccgggagacgcgccggcagccccggcaccgcagcggtcgcaggatggccgaggctatcagctgtactctgaactgtagttgccaaagtttcaaacccgggaaaataaaccaccgtcagtgtgaccaatgcaagcatggatgggtggcccacgctctaagtaagctaaggatcccccccatgtatccaacaagccaggtggagattgtccagtccaatgtagtgtttgatattagcagcctcatgctctatgggacccaggccatccccgttcgcctaaaaatcctactggaccggctcttcagtgtgttgaagcaagatgaggttctccagatcctccatgccttggactggacacttcaggattatatccgtggatacgtactgcaggatgcatcaggaaaggtgttggatcactggagcatcatgaccagtgaggaagaagtggccaccttgcagcagttccttcgttttggagagaccaaatctatagttgaactcatggcaattcaagagaaagaagagcaatccatcatcataccaccttccacagcaaatgtagatatcagggctttcatcgagagctgcagtcacaggagttctagcctccccactcctgtggacaaaggaaaccccagcagtatacacccctttgagaacctcataagcaacatgactttcatgctgcctttccagttcttcaaccctctgcctcctgcactgatagggtcattgcccgaacaatatatgttggagcagggtcatgaccaaagtcaggaccccaaacaggaagtccatgggcccttccctgacagcagcttcttaacttccagttccacaccatttcaggttgaaaaagatcagtgtttaaactgtccggatgctattactaaaaaagaagacagcacccatttaagtgactccagctcatacaacattgtcactaagtttgaaaggacacagttatcccctgaggccaaagtgaagcctgagaggaatagccttggtacaaagaagggccgggtgttctgcactgcatgtgagaagaccttctatgacaaaggcaccctcaaaatccactacaatgccgtccacttgaagatcaagcataagtgcaccatcgaagggtgtaacatggtgttcagctccctaaggagccggaatcgccatagcgccaaccccaaccctcggctgcacatgccaatgaacagaaataaccgggacaaagacctcaggaacagcctgaacctggccagctctgagaactacaagtgcccaggtttcacagtgacgtccccagactgtaggcctcctcccagctaccctggttcaggagaggattccaaaggccaaccagccttcccaaacattgggcaaaatggtgtgctttttcccaacctaaagacagtccagccagtccttcctttctaccgcagtccagccacgcctgccgaggtagcaaacacgcctgggatactcccttccctcccgctgttgtcctcttcaatcccagaacagctcatttcaaacgaaatgccatttgatgcccttcccaagaagaaatccaggaagtccagtatgcctatcaaaatagagaaagaagctgtggaaatagctaatgagaaaagacacaacctcagctcagatgaagacatgcccctacaggtggtcagtgaagatgagcaggaggcctgcagtcctcagtcacacagagtatctgaggagcagcatgtacagtcaggaggcttagggaagcctttccctgaaggggagaggccctgccatcgtgaatcagtaattgagtccagtggagccatcagccaaacccctgagcaggccacacacaattcagagagggagactgagcagacaccagcattgatcatggtgccaagggaggtcgaggatggtggccatgaacactacttcacacctgggatggaaccccaagttcctttttctgactacatggaactgcagcagcgcctgctggctgggggactcttcagtgctttgtccaacaggggaatggcttttccttgtcttgaagattctaaagaactggagcacgtgggtcagcatgcattagcaaggcagatagaagaaaatcgcttccagtgtgacatctgcaagaagacctttaaaaatgcttgtagtgtgaaaattcatcacaagaatatgcatgtcaaagaaatgcacacatgcacagtggagggctgtaatgctacctttccctcccgcaggagcagagacagacacagctcaaacctaaacctccaccaaaaagcattgagccaggaagcattggagagtagtgaagatcatttccgtgcagcttaccttctgaaagatgtggctaaggaagcctatcaggatgtggcttttacacagcaagcctcccagacatctgtcatcttcaaaggaacaagtcgaatgggcagtctggtttacccaataacgcaagtccacagtgccagcctggagagctacaactctggccccttgagcgagggcaccatcctggatttgagcactacctcgagcatgaagtcagagagtagcagccattcttcctgggactctgacggggtgagtgaggaaggcactgtgcttatggaggacagtgatgggaactgtgaagggtcgagccttgtccctggggaagatgagtaccccatctgtgtcctgatggagaaggctgaccagagccttgctagcctgccttctgggttgcccataacctgtcatctctgccaaaagacatacagtaacaaagggacctttagggcccactacaaaactgtgcacctccggcagctccacaaatgcaaagtaccaggctgcaacaccatgttttcgtctgttcgcagtcgaaacagacacagccagaatcccaacctgcacaaaagcctggcctcatctccaagtcacctccagtaacaagatggcaaaccaagtatgctcagataagcttttttcataattcaggaataaagtagtccatagaaatgtttctgtttcatatcatttggggcgagtcaggcaaaagtatttgatttgactttatagttttccacagcacaatgagcaaaagacaaacctcgtgggaagatgacactggggcagcccttcctattatttttcttagcccaagaggtctttcactgatacaaggaaaacttgcagaaatgtgatttttcccagatttgtttacatgttccctgggacagatccaggtctgcagatcgacaccagtgggcccaggacctgggggtggctttaaatgaggcttgcagtgttaaaggtcttggataagaagggtcctggggaagaagactctgtggacaagataccagtccccaaaacagcattttcagttccttcttcaattagtttgaaatccagacctgagtttggaagactgattttttgagaccatccctgtgtttggagtggataattgtccctcccctcagccctgcaccagaggtctcatatgttaccccagggagttctcagaggattgggttggcctctaacatgttccttgttaattcttgttctgtaacatgcattcaagaagctaggggaaaaatatctcatgcacttaaataatggtcttcaatttaatttaaaaatattttgacaatatttaatttgtgcttatgtggtgtttggtgtgagtgcagatattgcactgtgtcacctctggatctctgctcagaagcagaacaagtgatgacctaaatgtcaaaatcactgctcgttttcatttggtgaacttcaaactctgttctttttggtcacctgtggaatgaatgcaagcatgattttggcaggaacatttgtacatattctgccgtagataatgtggttctgatggttgttgtgtattttcagtatcactggatccctcagtcttcaccgttttataaacgtataagattaggatgaacttttgaatttacttggtaggaaaaaaagtaggacattattgccatattgtatgtcttaatatttaacttattcggaaatatattccacactgttacatacattttccatggtagaaaggaagttcagtcagtcctgtggaatgaaaccatctcctaaaattcagcatttgcagcattctaaaagcctgtgtaggtacaaggacattgattttgtattcagaattcaagttaactatcttttaaattcgtggttgatgtaagtaataaaaaacattcttaaagttgagggttataagagagattatttctgtggtctaaaggttaaaaagccaacaacctgttaccaattatttcagctttttttgttttaataagtgtgacaacttaaaacttgtttctatttaaagtgaaatgtatctttcaactgtttagttacccagctgtttaatattccagtcttcccaaagtgaaaagatttgtatacaaatgttttctatgatttaataaaaatatatggcacaccaaaaaaaaaaaaaaa

SEQ ID NO: 16 - DNA, 1574 bp, Homo sapiens
atcgctacgcccacttggtggcctataaaggaagcgggcgaaccccggcagccctacacaacttggggcccctctcctctccagcccttctcctgtgtgcctgcctcctgccgccgccaccatgaccacctccatccgccagttcacctcctccagctccatcaagggctcctccggcctggggggcggctcgtcccgcacctcctgccggctgtctggcggcctgggtgccggctcctgcaggctgggatctgctggcggcctgggcagcaccctcgggggtagcagctactccagctgctacagctttggctctggtggtggctatggcagcagctttgggggtgttgatgggctgctggctggaggtgagaaggccaccatgcagaacctcaatgaccgcctggcctcctacctggacaaggtgcgtgccctggaggaggccaacactgagctggaggtgaagatccgtgactggtaccagaggcaggccccggggcccgcccgtgactacagccagtactacaggacaattgaggagctgcagaacaagatcctcacagccaccgtggacaatgccaacatcctgctacagattgacaatgcccgtctggctgctgatgacttccgcaccaagtttgagacagagcaggccctgcgcctgagtgtggaggccgacatcaatggcctgcgcagggtgctggatgagctgaccctggccagagccgacctggagatgcagattgagaacctcaaggaggagctggcctacctgaagaagaaccacgaggaggagatgaacgccctgcgaggccaggtgggtggtgagatcaatgtggagatggacgctgccccaggcgtggacctgagccgcatcctcaacgagatgcgtgaccagtatgagaagatggcagagaagaaccgcaaggatgccgaggattggttcttcagcaagacagaggaactgaaccgcgaggtggccaccaacagtgagctggtgcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgaccacccgtcaggtgcgtaccattgtggaagaggtccaggatggcaaggtcatctcctcccgcgagcaggtccaccagaccacccgctgaggactcagctaccccggccggccacccaggaggcagggaggcagccgccccatctgccccacagtctccggcctctccagcctcagccccctgcttcagtcccttccccatgcttccttgcctgatgacaataaagcttgttgactcagctatg

SEQ ID NO: 17 - DNA, 2052 bp, Homo sapiens
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 18 - DNA, 2323 bp, Homo sapiens
ttgggcggggcgtaaaagccgggcgttcggaggacccagcaattagtctgatttccgcccacctttccgagcgggaaggagagccacaaagcgcgcatgcgcgcggatcaccgcaggctcctgtgccttgggcttgagctttgtggcagttaatggcttttctgcacgtatctctggtgtttacttgagaagcctggctgtgtccttgctgtaggagccggagtagctcagagtgatcttgtctgaggaaaggccagccccacttggggttaataaaccgcgatgggtgaaccctcaggaggctatacttacacccaaacgtcgatattccttttccacgctaagattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 19 - DNA, 1957 bp, Homo sapiens
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 20 - DNA, 2102 bp, Homo sapiens
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 21 - DNA, 2108 bp, Homo sapiens
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 22 - DNA, 2226 bp, Homo sapiens
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 23 - DNA, 1460 bp, Homo sapiens
gggcggggggcagggctccgggggactgggcgggccatggcggaggacggcgaggaggcggagttccacttcgcggcgctctatataagtgggcagtggccgcgactgcgcgcagacactgaccttcagcgcctcggctccagcgccatggcgccctccaggaagttcttcgttgggggaaactggaagatgaacgggcggaagcagagtctgggggagctcatcggcactctgaacgcggccaaggtgccggccgacaccgaggtggtttgtgctccccctactgcctatatcgacttcgcccggcagaagctagatcccaagattgctgtggctgcgcagaactgctacaaagtgactaatggggcttttactggggagatcagccctggcatgatcaaagactgcggagccacgtgggtggtcctggggcactcagagagaaggcatgtctttggggagtcagatgagctgattgggcagaaagtggcccatgctctggcagagggactcggagtaatcgcctgcattggggagaagctagatgaaagggaagctggcatcactgagaaggttgttttcgagcagacaaaggtcatcgcagataacgtgaaggactggagcaaggtcgtcctggcctatgagcctgtgtgggccattggtactggcaagactgcaacaccccaacaggcccaggaagtacacgagaagctccgaggatggctgaagtccaacgtctctgatgcggtggctcagagcacccgtatcatttatggaggctctgtgactggggcaacctgcaaggagctggccagccagcctgatgtggatggcttccttgtgggtggtgcttccctcaagcccgaattcgtggacatcatcaatgccaaacaatgagccccatccatcttccctacccttcctgccaagccagggactaagcagcccagaagcccagtaactgccctttccctgcatatgcttctgatggtgtcatctgctccttcctgtggcctcatccaaactgtatcttcctttactgtttatatcttcaccctgtaatggttgggaccaggccaatcccttctccacttactataatggttggaactaaacgtcaccaaggtggcttctccttggctgagagatggaaggcgtggtgggatttgctcctgggttccctaggccctagtgagggcagaagagaaaccatcctctcccttcttacaccgtgaggccaagatcccctcagaaggcaggagtgctgccctctcccatggtgcccgtgcctctgtgctgtgtatgtgaaccacccatgtgagggaataaacctggcactaggtcttgtggtttgtctgccttcactggacttgcccagataatcttcctttttgaggcagctatataaatgatcatttgtgcaagaaaaaaaaaaaaacaagaacaggtttctataacaa

SEQ ID NO: 24 - DNA, 1602 bp, Homo sapiens
ctcgccggcgtccgcgtccccgcgccgagctgctcgggctccctgagccccagatctgaccccttcccttcggcaacctgaacgactcccgccttccacggaagggaccgagcccgtgccaaacaggctgagcgatttgggagtgaggagccatcctaccgctttccccaacctggaaacagcaaagcgcaaggcctctgagtcagttaggtctctgccacccacgggcaaaggatgctctcctccatcctccttcctccctccaccgaaatcggagagccgcgggcctgatccaaagaggcatccccttctcgttcattccccagaggcctcaatacaaaccccaggagttggcccctctccttttgctacaaatccttgccttgcaaaggggagaggtggtttgtgctccccctactgcctatatcgacttcgcccggcagaagctagatcccaagattgctgtggctgcgcagaactgctacaaagtgactaatggggcttttactggggagatcagccctggcatgatcaaagactgcggagccacgtgggtggtcctggggcactcagagagaaggcatgtctttggggagtcagatgagctgattgggcagaaagtggcccatgctctggcagagggactcggagtaatcgcctgcattggggagaagctagatgaaagggaagctggcatcactgagaaggttgttttcgagcagacaaaggtcatcgcagataacgtgaaggactggagcaaggtcgtcctggcctatgagcctgtgtgggccattggtactggcaagactgcaacaccccaacaggcccaggaagtacacgagaagctccgaggatggctgaagtccaacgtctctgatgcggtggctcagagcacccgtatcatttatggaggctctgtgactggggcaacctgcaaggagctggccagccagcctgatgtggatggcttccttgtgggtggtgcttccctcaagcccgaattcgtggacatcatcaatgccaaacaatgagccccatccatcttccctacccttcctgccaagccagggactaagcagcccagaagcccagtaactgccctttccctgcatatgcttctgatggtgtcatctgctccttcctgtggcctcatccaaactgtatcttcctttactgtttatatcttcaccctgtaatggttgggaccaggccaatcccttctccacttactataatggttggaactaaacgtcaccaaggtggcttctccttggctgagagatggaaggcgtggtgggatttgctcctgggttccctaggccctagtgagggcagaagagaaaccatcctctcccttcttacaccgtgaggccaagatcccctcagaaggcaggagtgctgccctctcccatggtgcccgtgcctctgtgctgtgtatgtgaaccacccatgtgagggaataaacctggcactaggtcttgtggtttgtctgccttcactggacttgcccagataatcttcctttttgaggcagctatataaatgatcatttgtgcaagaaaaaaaaaaaaacaagaacaggtttctataacaaaaaaaaaa

SEQ ID NO: 25 - DNA, 1366 bp, Homo sapiens
gcgcagacactgaccttcagcgcctcggctccagcgccatggcgccctccaggaagttcttcgttgggggaaactggaagatgaacgggcggaagcagagtctgggggagctcatcggcactctgaacgcggccaaggtgccggccgacaccgaggtggtttgtgctccccctactgcctatatcgacttcgcccggcagaagctagatcccaagattgctgtggctgcgcagaactgctacaaagtgactaatggggcttttactggggagatcagccctggcatgatcaaagactgcggagccacgtgggtggtcctggggcactcagagagaaggcatgtctttggggagtcagatgagctgattgggcagaaagtggcccatgctctggcagagggactcggagtaatcgcctgcattggggagaagctagatgaaagggaagctggcatcactgagaaggttgttttcgagcagacaaaggtcatcgcagataacgtgaaggactggagcaaggtcgtcctggcctatgagcctgtgtgggccattggtactggcaagactgcaacaccccaacaggcccaggaagtacacgagaagctccgaggatggctgaagtccaacgtctctgatgcggtggctcagagcacccgtatcatttatggaggctctgtgactggggcaacctgcaaggagctggccagccagcctgatgtggatggcttccttgtgggtggtgcttccctcaagcccgaattcgtggacatcatcaatgccaaacaatgagccccatccatcttccctacccttcctgccaagccagggactaagcagcccagaagcccagtaactgccctttccctgcatatgcttctgatggtgtcatctgctccttcctgtggcctcatccaaactgtatcttcctttactgtttatatcttcaccctgtaatggttgggaccaggccaatcccttctccacttactataatggttggaactaaacgtcaccaaggtggcttctccttggctgagagatggaaggcgtggtgggatttgctcctgggttccctaggccctagtgagggcagaagagaaaccatcctctcccttcttacaccgtgaggccaagatcccctcagaaggcaggagtgctgccctctcccatggtgcccgtgcctctgtgctgtgtatgtgaaccacccatgtgagggaataaacctggcactaggtcttgtggtttgtctgccttcactggacttgcccagataatcttcctttttgaggcagctatataaatgatcatttgtgcaagaaaaaaaaaaaaacaagaacaggtttctataacaaaaaaaaaaaaaaaaa

SEQ ID NO: 26 - DNA, 1561 bp, Homo sapiens
gcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataataaaaaaaaa

SEQ ID NO: 27 - DNA, 3309 bp, Homo sapiens
ttcagcccctctcccgggctgcgcctccgcactccgggcccgggcagaagggggtgcgcctcggccccaccacccagggagcagccgagctgaaaggccgggaaccgcggcttgcggggaccacagctcccgaaagcgacgttcggccaccggaggagcgggagccaagcaggcggagctcggcgggagaggtgcgggccgaatccgagccgagcggagaggaatccggcagtagagagcggactccagccggcggaccctgcagccctcgcctgggacagcggcgcgctgggcaggcgcccaagagagcatcgagcagcggaacccgcgaagccggcccgcagccgcgacccgcgcagcctgccgctctcccgccgccggtccgggcagcatgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcctgacgcgggagccatgcgccccctccgccctgccactcactaggcccccacttgcctcttccttgaagaactgcaggccctggcctcccctgccaccaggccacctccccagcattccagcccctctggtcgctcctgcccacggagtcgtggggtgtgctgggagctccactctgcttctctgacttctgcctggagacttagggcaccaggggtttctcgcataggacctttccaccacagccagcacctggcatcgcaccattctgactcggtttctccaaactgaagcagcctctccccaggtccagctctggaggggagggggatccgactgctttggacctaaatggcctcatgtggctggaagatcctgcgggtggggcttggggctcacacacctgtagcacttactggtaggaccaagcatcttgggggggtggccgctgagtggcaggggacaggagtccactttgtttcgtggggaggtctaatctagatatcgacttgtttttgcacatgtttcctctagttctttgttcatagcccagtagaccttgttacttctgaggtaagttaagtaagttgattcggtatccccccatcttgcttccctaatctatggtcgggagacagcatcagggttaagaagactttttttttttttttttaaactaggagaaccaaatctggaagccaaaatgtaggcttagtttgtgtgttgtctcttgagtttgtcgctcatgtgtgcaacagggtatggactatctgtctggtggccccgtttctggtggtctgttggcaggctggccagtccaggctgccgtggggccgccgcctctttcaagcagtcgtgcctgtgtccatgcgctcagggccatgctgaggcctgggccgctgccacgttggagaagcccgtgtgagaagtgaatgctgggactcagccttcagacagagaggactgtagggagggcggcaggggcctggagatcctcctgcagaccacgcccgtcctgcctgtggcgccgtctccaggggctgcttcctcctggaaattgacgaggggtgtcttgggcagagctggctctgagcgcctccatccaaggccaggttctccgttagctcctgtggccccaccctgggccctgggctggaatcaggaatattttccaaagagtgatagtcttttgcttttggcaaaactctacttaatccaatgggtttttccctgtacagtagattttccaaatgtaataaactttaatataaagtagtcctgtgaatgccactgccttcgcttcttgcctctgtgctgtgtgtgacgtgaccggacttttctgcaaacaccaacatgttgggaaacttggctcgaatctctgtgccttcgtctttcccatggggagggattctggttccagggtccctctgtgtatttgcttttttgttttggctgaaattctcctggaggtcggtaggttcagccaaggttttataaggctgatgtcaatttctgtgttgccaagctccaagccccatcttctaaatggcaaaggaaggtggatggccccagcacagcttgacctgaggctgtggtcacagcggaggtgtggagccgaggcctaccccgcagacaccttggacatcctcctcccacccggctgcagaggccagaggcccccagcccagggctcctgcacttacttgcttatttgacaacgtttcagcgactccgttggccactccgagaggtgggccagtctgtggatcagagatgcaccaccaagccaagggaacctgtgtccggtattcgatactgcgactttctgcctggagtgtatgactgcacatgactcgggggtggggaaaggggtcggctgaccatgctcatctgctggtccgtgggacggtgcccaagccagaggctgggttcatttgtgtaacgacaataaacggtacttgtcatttcgggcaaaaaaaaaaaaaaaaaa

SEQ ID NO: 28 - DNA, 3217 bp, Homo sapiens
ggccgggagacctggcggagctgggggtggggggccagtttttgcaacggctaaggaagggcctgtgggtttattataaggcggagctcggcgggagaggtgcgggccgaatccgagccgagcggagaggaatccggcagtagagagcggactccagccggcggaccctgcagccctcgcctgggacagcggcgcgctgggcaggcgcccaagagagcatcgagcagcggaacccgcgaagccggcccgcagccgcgacccgcgcagcctgccgctctcccgccgccggtccgggcagcatgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcctgacgcgggagccatgcgccccctccgccctgccactcactaggcccccacttgcctcttccttgaagaactgcaggccctggcctcccctgccaccaggccacctccccagcattccagcccctctggtcgctcctgcccacggagtcgtggggtgtgctgggagctccactctgcttctctgacttctgcctggagacttagggcaccaggggtttctcgcataggacctttccaccacagccagcacctggcatcgcaccattctgactcggtttctccaaactgaagcagcctctccccaggtccagctctggaggggagggggatccgactgctttggacctaaatggcctcatgtggctggaagatcctgcgggtggggcttggggctcacacacctgtagcacttactggtaggaccaagcatcttgggggggtggccgctgagtggcaggggacaggagtccactttgtttcgtggggaggtctaatctagatatcgacttgtttttgcacatgtttcctctagttctttgttcatagcccagtagaccttgttacttctgaggtaagttaagtaagttgattcggtatccccccatcttgcttccctaatctatggtcgggagacagcatcagggttaagaagactttttttttttttttttaaactaggagaaccaaatctggaagccaaaatgtaggcttagtttgtgtgttgtctcttgagtttgtcgctcatgtgtgcaacagggtatggactatctgtctggtggccccgtttctggtggtctgttggcaggctggccagtccaggctgccgtggggccgccgcctctttcaagcagtcgtgcctgtgtccatgcgctcagggccatgctgaggcctgggccgctgccacgttggagaagcccgtgtgagaagtgaatgctgggactcagccttcagacagagaggactgtagggagggcggcaggggcctggagatcctcctgcagaccacgcccgtcctgcctgtggcgccgtctccaggggctgcttcctcctggaaattgacgaggggtgtcttgggcagagctggctctgagcgcctccatccaaggccaggttctccgttagctcctgtggccccaccctgggccctgggctggaatcaggaatattttccaaagagtgatagtcttttgcttttggcaaaactctacttaatccaatgggtttttccctgtacagtagattttccaaatgtaataaactttaatataaagtagtcctgtgaatgccactgccttcgcttcttgcctctgtgctgtgtgtgacgtgaccggacttttctgcaaacaccaacatgttgggaaacttggctcgaatctctgtgccttcgtctttcccatggggagggattctggttccagggtccctctgtgtatttgcttttttgttttggctgaaattctcctggaggtcggtaggttcagccaaggttttataaggctgatgtcaatttctgtgttgccaagctccaagccccatcttctaaatggcaaaggaaggtggatggccccagcacagcttgacctgaggctgtggtcacagcggaggtgtggagccgaggcctaccccgcagacaccttggacatcctcctcccacccggctgcagaggccagaggcccccagcccagggctcctgcacttacttgcttatttgacaacgtttcagcgactccgttggccactccgagaggtgggccagtctgtggatcagagatgcaccaccaagccaagggaacctgtgtccggtattcgatactgcgactttctgcctggagtgtatgactgcacatgactcgggggtggggaaaggggtcggctgaccatgctcatctgctggtccgtgggacggtgcccaagccagaggctgggttcatttgtgtaacgacaataaacggtacttgtcatttcgggcaaaaaaaaaaaaaaaaaa

SEQ ID NO: 29 - DNA, 2010 bp, Homo sapiens
ggcagcgactgcgccccgtcccggcgccgcgctcgtccgcagaggaggcggcccggcccgggcagctgcggctcgggatccgtcgaggggaggccgagcttgccaagctggcgcccagcggggtcatggtgcccggcgcccgcggcggcggcgcactggcgcgggctgccgggcggggcctcctggctttgctgctcgcggtctccgccccgctccggctgcaggcggaggagctgggtgatggctgtggacacctagtgacttatcaggatagtggcacaatgacatctaagaattatcccgggacctaccccaatcacactgtttgcgaaaagacaattacagtaccaaaggggaaaagactgattctgaggttgggagatttggatatcgaatcccagacctgtgcttctgactatcttctcttcaccagctcttcagatcaatatggtccatactgtggaagtatgactgttcccaaagaactcttgttgaacacaagtgaagtaaccgtccgctttgagagtggatcccacatttctggccggggttttttgctgacctatgcgagcagcgaccatccagatttaataacatgtttggaacgagctagccattatttgaagacagaatacagcaaattctgcccagctggttgtagagacgtagcaggagacatttctgggaatatggtagatggatatagagatacctctttattgtgcaaagctgccatccatgcaggaataattgctgatgaactaggtggccagatcagtgtgcttcagcgcaaagggatcagtcgatatgaagggattctggccaatggtgttctttcgagggatggttccctgtcagacaagcgatttctgtttacctccaatggttgcagcagatccttgagttttgaacctgacgggcaaatcagagcttcttcctcatggcagtcggtcaatgagagtggagaccaagttcactggtctcctggccaagcccgacttcaggaccaaggcccatcatgggcttcgggcgacagtagcaacaaccacaaaccacgagagtggctggagatcgatttgggggagaaaaagaaaataacaggaattaggaccacaggatctacacagtcgaacttcaacttttatgttaagagttttgtgatgaacttcaaaaacaataattctaagtggaagacctataaaggaattgtgaataatgaagaaaaggtgtttcagggtaactctaactttcgggacccagtgcaaaacaatttcatccctcccatcgtggccagatatgtgcgggttgtcccccagacatggcaccagaggatagccttgaaggtggagctcattggttgccagattacacaaggtaatgattcattggtgtggcgcaagacaagtcaaagcaccagtgtttcaactaagaaagaagatgagacaatcacaaggcccatcccctcggaagaaacatccacaggaataaacattacaacggtggctattccattggtgctccttgttgtcctggtgtttgctggaatggggatctttgcagcctttagaaagaagaagaagaaaggaagtccgtatggatcagcagaggctcagaaaacagactgttggaagcagattaaatatccctttgccagacatcagtcagctgagtttaccatcagctatgataatgagaaggagatgacacaaaagttagatctcatcacaagtgatatggcaggttaactccgttgactgccaaaatagcatccccaacgtgcagccctccgcatctatcagcaggttgccccggatggatctcagagatgaggatcggaacaccatgttctttcccaccctaacaacaacaaagggcagtaaattaaagtactctttgtaaggtacagttaccgattaatctagagataaaatattttcttaaaaatatatttcattaaacacctatgctgtctctataaaaaaaaaaaaaaaaaaaaaaaaaaaaaaaaaa

SEQ ID NO: 30 - DNA, 1572 bp, Homo sapiens
gtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccttgaatccactcgccagcccgccgccctctgccgccgcaccctgcacacccgcccctctcctgtgccaggaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcaccttgctgaagcccaacatggtcaccccaggccatgcttgcactcagaagttttctcatgaggagattgccatggcgaccgtcacagcgctgcgccgcacagtgccccccgctgtcactgggatcaccttcctgtctggaggccagagtgaggaggaggcgtccatcaacctcaatgccattaacaagtgccccctgctgaagccctgggccctgaccttctcctacggccgagccctgcaggcctctgccctgaaggcctggggcgggaagaaggagaacctgaaggctgcgcaggaggagtatgtcaagcgagccctggccaacagccttgcctgtcaaggaaagtacactccgagcggtcaggctggggctgctgccagcgagtccctcttcgtctctaaccacgcctattaagcggaggtgttcccaggctgcccccaacactccaggccctgccccctcccactcttgaagaggaggccgcctcctcggggctccaggctggcttgcccgcgctctttcttccctcgtgacagtggtgtgtggtgtcgtctgtgaatgctaagtccatcaccctttccggcacactgccaaataaacagctatttaagggggaaaaaaaaaaaaaaaaaaaaaaaaaaaaaaaaaaaaaaaaaaaaaaaaaa

SEQ ID NO: 31 - DNA, 1594 bp, Homo sapiens
cttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaagatttatttctcttgacaaccaagggcctccgtctggatttccaaggaagaatttcctctgaagcaccggaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcaccttgctgaagcccaacatggtcaccccaggccatgcttgcactcagaagttttctcatgaggagattgccatggcgaccgtcacagcgctgcgccgcacagtgccccccgctgtcactgggatcaccttcctgtctggaggccagagtgaggaggaggcgtccatcaacctcaatgccattaacaagtgccccctgctgaagccctgggccctgaccttctcctacggccgagccctgcaggcctctgccctgaaggcctggggcgggaagaaggagaacctgaaggctgcgcaggaggagtatgtcaagcgagccctggccaacagccttgcctgtcaaggaaagtacactccgagcggtcaggctggggctgctgccagcgagtccctcttcgtctctaaccacgcctattaagcggaggtgttcccaggctgcccccaacactccaggccctgccccctcccactcttgaagaggaggccgcctcctcggggctccaggctggcttgcccgcgctctttcttccctcgtgacagtggtgtgtggtgtcgtctgtgaatgctaagtccatcaccctttccggcacactgccaaataaacagctatttaagggggaaaaaaaaaaaaaaaaaaaaaaaaaaaaaaaaaaaaaaaaaaaaaaaaaa

SEQ ID NO: 32 - DNA, 1478 bp, Homo sapiens
aaaagggcaggggtcattagagaagatcggggacacatgtggggcgggcaggagctgccttataaccagcccgggaacccctagctcactcgctgctgaccaggctctgccggctccttcggcctcgccgcaggaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcaccttgctgaagcccaacatggtcaccccaggccatgcttgcactcagaagttttctcatgaggagattgccatggcgaccgtcacagcgctgcgccgcacagtgccccccgctgtcactgggatcaccttcctgtctggaggccagagtgaggaggaggcgtccatcaacctcaatgccattaacaagtgccccctgctgaagccctgggccctgaccttctcctacggccgagccctgcaggcctctgccctgaaggcctggggcgggaagaaggagaacctgaaggctgcgcaggaggagtatgtcaagcgagccctggccaacagccttgcctgtcaaggaaagtacactccgagcggtcaggctggggctgctgccagcgagtccctcttcgtctctaaccacgcctattaagcggaggtgttcccaggctgcccccaacactccaggccctgccccctcccactcttgaagaggaggccgcctcctcggggctccaggctggcttgcccgcgctctttcttccctcgtgacagtggtgtgtggtgtcgtctgtgaatgctaagtccatcaccctttccggcacactgccaaataaacagctatttaagggggaaaaaaaaaaaaaaaaaaaaaaaaaaaa

SEQ ID NO: 33 - DNA, 2353 bp, Homo sapiens
cctagcttggcgcggaatccgtgaattgcccgcggcccgagggtgcagctcccggactgactggctctgcccttccccatggacgcctcctctagcccgtggaatccaaccccggctcctgtcagcagccctcccctgctgctccccatccctgccatcgtcttcatcgctgtgggcatctatttgttgctgctgggtctagtcctgctgactaggaactgcctgctggcccagggctgctgcgcggacggtagctccccctgcaggaagcaaggttcctccgggcccccagactgctgctggacctgtgcagaagcctgcaactttcctctgcctagcccggcccacttcctggatgcttgctgcccccagcccaccagagctgactgggcacctcgctgcccccgctgctgcccactctgcgactgtgcctgtacgtgccagctccccgactgccagagcctcaactgtctctgcttcgagatcaagctccgatgaggacccagggcccctgccctctggggagcggccagcccccagggcccatgtgccctcctccctgaagagcctttccccacgccactggaaccacagatggcctgccgagcacccaggcctgggaactggaagtggcagcgcagggcctggctccctgcagggcaggactcttggccggctggacggcagctcctctggagggccagaaaagagaggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtccctggccaaagatttatttctcttgacaaccaagggcctccgtctggatttccaaggaagaatttcctctgaagcaccggaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcaccttgctgaagcccaacatggtcaccccaggccatgcttgcactcagaagttttctcatgaggagattgccatggcgaccgtcacagcgctgcgccgcacagtgccccccgctgtcactgggatcaccttcctgtctggaggccagagtgaggaggaggcgtccatcaacctcaatgccattaacaagtgccccctgctgaagccctgggccctgaccttctcctacggccgagccctgcaggcctctgccctgaaggcctggggcgggaagaaggagaacctgaaggctgcgcaggaggagtatgtcaagcgagccctggccaacagccttgcctgtcaaggaaagtacactccgagcggtcaggctggggctgctgccagcgagtccctcttcgtctctaaccacgcctattaagcggaggtgttcccaggctgcccccaacactccaggccctgccccctcccactcttgaagaggaggccgcctcctcggggctccaggctggcttgcccgcgctctttcttccctcgtgacagtggtgtgtggtgtcgtctgtgaatgctaagtccatcaccctttccggcacactgccaaataaacagctatttaagggggaaaaaaaaaaaaaaaaaaaaaaaaaaaaaaaaaaaaaaaaaaaaaaaaaa

SEQ ID NO: 34 - DNA, 3167 bp, Homo sapiens
aaaccttcggcggccggcgctgtgcggcgggcgcggttgcgcgcggcttggggcaaatacttctcaccactgcatgaatggacatttgaaagtgccatagccaaacacttgcaagcatggagacctcatcaatgctttcctcattgaatgatgagtgtaaatctgacaactacattgagcctcactacaaggaatggtatcgagtagccattgatattctgattgaacacgggttagaagcataccaagaatttcttgtccaggaacgagtttcagactttcttgctgaggaagaaattaattatattttgaaaaatgtccagaaagttgcacaaagcacagcacatggtactgatgattcctgtgatgataccttatcttcagggacctactggcctgttgagtctgatgtggaagctccaaatcttgacttaggctggccatatgtgatgcccggactcttagggggcacccatatagatctcctttttcatccaccaagagcacatctacttacgataaaagaaactattcggaagatgataaaagaagcaagaaaggtcattgctttagtgatggatatatttacagatgtggacattttcaaagaaatcgttgaggcatcaactcgaggagtatctgtttacattctgcttgatgagtccaattttaatcattttctaaatatgactgagaaacaaggttgttcagttcagcgtctcaggaatattcgagtgcgaacagtaaaaggccaagattatctttcaaaaacaggggcaaaattccatggaaaaatggaacagaaatttttgttagttgactgccagaaagtgatgtacggttcttacagttatatgtggtcatttgagaaagctcacctcagcatggttcagataattacaggacaacttgttgagtcctttgatgaagaatttagaactctctatgccagatcctgtgtccctagttcatttgctcaggaagaatcagcaagggtgaagcatggaaaagccctctgggaaaatggcacttaccagcattcggtgtcttcattagcatctgtttccagccagagaaacctttttggtagacaagacaagattcataaactagattccagttacttcaaaaacagagggatatatactttaaatgaacatgacaaatataacataagaagtcacggatacaaacctcattttgttcctaactttaatggtccaaacgcaatacgtcagtttcaacccaatcagataaatgaaaattggaaaaggcatagttatgctggggaacagccagaaacagtgccatacctcctgcttaatagggctctgaatagaaccaataatccacctggtaattggaaaaagccatctgatagtctcagtgtggcgtcctcatcacgggaaggctatgtaagccaccacaacacacctgcccagagttttgccaatcggcttgcgcagagaaaaacaacaaatcttgcagacaggaattcaaatgttcggaggtcttttaatgggacagataaccatatccgctttttgcaacaacgaatgccaacccttgaacataccacaaagtcattcctacgtaactggagaattgaatcctacttaaatgatcattcagaagctacaccggactcaaatggatcagctttaggtgaccgatttgagggctatgataatcctgagaatttgaaggccaatgccctttatactcattctcggcttcgttcctctttagtatttaaacccactttacctgagcaaaaggaagttaacagttgtacaactggctcctcaaattcaactatcattggttctcagggaagtgagacacctaaagaggtcccagacacccctacgaatgtacagcatttgacagacaaacccttgccagaatcaatccccaagctcccattgcagtcagaggcaccaaaaatgcacaccttgcaggttcctgaaaaccactcagtagccttaaaccaaactacaaatggccatactgaatcaaataactatatatataaaaccttgggtgtaaataagcagacagaaaatctaaagaatcaacagactgagaatctacttaaaaggcgaagtttcccgttatttgacaactcaaaagccaacttagatcctggaaatagtaagcattatgtatatagtacacttaccaggaatcgagttagacaaccagaaaagcccaaagaagatttgctgaaaagttctaaaagcatgcacaatgtgactcataacttggaggaggatgaggaggaagttaccaagagaaactctccaagtggcactactaccaaatcagtttccattgctgctttacttgatgtgaataaagaggaatctaacaaagaacttgcttcaaagaaggaagttaagggttccccaagttttttgaaaaaggggtctcagaagttaaggtcattacttagccttaccccagataagaaagaaaatctatccaaaaataaagcacctgccttttatagattgtgtagtagctctgacacattagtttctgagggtgaagaaaatcaaaaaccaaagaaatcagacacaaaagttgattcatctcctagaagaaagcattcttcctcatcgaattctcaaggcagcatccacaagagtaaggaagatgtaacagttagcccatctcaagagataaatgctccaccagatgaaaataaaagaacaccttctccaggtccagttgaaagcaagttcttggaaagggcaggagatgcctctgccccaagatttaacactgaacagatccaataccgagattcaagggagattaatgcagttgttacccctgaaagaagacctacttcttctccaaggccaacgtccagtgagcttctacgatctcattcaactgatcggcgtgtttacagtcgttttgagccgttttgtaagattgagagctctattcagccaacaagcaacatgccaaataccagtataaatcgcccagaaataaaatctgcgactatgggcaacagttatggcaggtctagtccattgcttaattacaacactggtgtttatcgctcatatcaacccaatgagaacaagtttcgaggatttatgcaaaagtttggaaactttatacacaaaaataaatagctattaaaatgcaaa

SEQ ID NO: 35 - DNA, 3318 bp, Homo sapiens
ggaggccgagctcggctgggcttggcgaggctgcggcgcggccaccggcgggagtgcagcggccactgtacccagagattcaaaaccccaaacccgggacttgggggcgctgagccgggccgggaagcagagcctggtcgtgaggaacagccgcccgttgctgtctgcccctttgcggacagcgtctccctcgactccgcttaggaagtggtgggggcggcgtggcccccgtcgggaggcgttcgaacgcccgctaggagagagaaaggattcccctgtgcttggagcccgcactcgggcgcggagggagcggcggcaggctctcgctttcggcaccatgggctgcacgctgagcgccgaggacaaggcggcggtggagcggagtaagatgatcgaccgcaacctccgtgaggacggcgagaaggcggcgcgcgaggtcaagctgctgctgctcggtgctggtgaatctggtaaaagtacaattgtgaagcagatgaaaattatccatgaagctggttattcagaagaggagtgtaaacaatacaaagcagtggtctacagtaacaccatccagtcaattattgctatcattagggctatggggaggttgaagatagactttggtgactcagcccgggcggatgatgcacgccaactctttgtgctagctggagctgctgaagaaggctttatgactgcagaacttgctggagttataaagagattgtggaaagatagtggtgtacaagcctgtttcaacagatcccgagagtaccagcttaatgattctgcagcatactatttgaatgacttggacagaatagctcaaccaaattacatcccgactcaacaagatgttctcagaactagagtgaaaactacaggaattgttgaaacccattttactttcaaagatcttcattttaaaatgtttgatgtgggaggtcagagatctgagcggaagaagtggattcattgcttcgaaggagtgacggcgatcatcttctgtgtagcactgagtgactacgacctggttctagctgaagatgaagaaatgaaccgaatgcatgaaagcatgaaattgtttgacagcatatgtaacaacaagtggtttacagatacatccattatactttttctaaacaagaaggatctctttgaagaaaaaatcaaaaagagccctctcactatatgctatccagaatatgcaggatcaaacacatatgaagaggcagctgcatatattcaatgtcagtttgaagacctcaataaaagaaaggacacaaaggaaatatacacccacttcacatgtgccacagatactaagaatgtgcagtttgtttttgatgctgtaacagatgtcatcataaaaaataatctaaaagattgtggtctcttttaagttttgcagttcatggtaaaatgcattttcaaaccaaatgagtacttatatatggatctctgtagactagagtcttgcagcaacacagaatgtaatataaggcaaatgcatctgggacttgaccaaagttgttctgttttgtttttttaactgaaagtaacagaaggacctttcttaaatgtgacagatggtcctgcagtgtgaaactgaaggacagtgttaaagctgggctctagtatattgatgatttctgcataagtgtaaatatgcaaatgtatgtatacatgtatttatgactttagttttcgacattatttttaggttttaagagtggcaacttaggattttagggtgatggctttggaaataacataaatataccttgtactgaatgacagactattactacgtttgccagttttaaacagctttatttatgttcatgtcctgtaaatttttaagtacagtaattaatattaggaaacattacagcccttatctagattatatgtatacttgtattaataaaaatgttatttgtacaaacattgcacagactattttaataacatgatttgttctttaaattttatgtgttttattgaaatgttcttaagatgaatacacctgcctttggatcaactatttaaacattgtatgcattttgatttttcctactttaagaaaataaaataatttaattttacattagattccacgttagatttggtttgaaaaactaaaatttcagatttctgaggatatactgtcttagacttattgtacacacttagtttttattcacttgttttcactctgaattttaatatttggctgatatgaatgcattgcctcaaaggtgatgtcatcttaatttttattcactttaaataactacatttttgtttataactaagtttggagggatcctaagagcatttttgtgggtaaaaaaaaaacctgtggacataatgaattttgagacattgattggtgaggcttttatttcccttgaggagtctcttgtacctagcatacatgatagctccttgttgggaagataacaagaaggatctttgaatactctattgctgatataatgcaagatttaaatttatacatataactaatttcaaatgtaattatcacactatgttaaaattacttttttcccttagataattcaaatttctccacttgcttgagattatatcatttctttttcaattatactattatttctgagaatgaaatggacgattacacttagaaaatgagtaatagtgtttaataagtcagtgattatatgtgtgctcaaataagtgttatgtatcagctagatactgagctttgatagaataattttcttttgattattcatgatgtgtcatctctgaccttgtttcagcaaagtaaacagcactccccaccccaccctcctttttttactcatcttggaaaaggttagtctttcagtacacgttgctggtaagtagtttccaagttacgtgttgtcactgggttgaagtatatttgtgtgtgtgtgtgtgtgtgtgtgtgtgtgtgtaaccataaactatattcatatctgtttcatttggaggattttcttctttgtaatgtaaagaaattcaaagttatcaaagttccttaaatgtgttagtttagattctttatgtgcctttcatgaaagatatgttttcattaattttactggtggacctgtaatatccacattgtgaagctgtgtatgaaattcaactataatatgaataaatttgaatcatgagaattatgggttaaaaagccacaaagaagcacatattggtgaccatcattaatgaaatcctgaactttattctgtgtaattgtgttaataaatcctaataaatttaaatttttaaaattttacaaac

SEQ ID NO: 36 - DNA, 906 bp, Homo sapiens
acagaaggacgaaccagtgagctaagctgcggggcgcgggctcggccggggcaccggtgagtcgccggcgctgcagagggaggcggcactggtctcgacgtggggcggccagcgatgaagccgcccagttcaatacaaacaagtgagtttgactcatcagatgaagagcctattgaagatgaacagactccaattcatatatcatggctatctttgtcacgagtgaattgttctcagtttctcggtttatgtgctcttccaggttgtaaatttaaagatgttagaagaaatgtccaaaaagatacagaagaactaaagagctgtggtatacaagacatatttgttttctgcaccagaggggaactgtcaaaatatagagtcccaaaccttctggatctctaccagcaatgtggaattatcacccatcatcatccaatcgcagatggagggactcctgacatagccagctgctgtgaaataatggaagagcttacaacctgccttaaaaattaccgaaaaaccttaatacactgctatggaggacttgggagatcttgtcttgtagctgcttgtctcctactatacctgtctgacacaatatcaccagagcaagccatagacagcctgcgagacctaagaggatccggggcaatacagaccatcaagcaatacaattatcttcatgagtttcgggacaaattagctgcacatctatcatcaagagattcacaatcaagatctgtatcaagataaaggaattcaaatagcatatatatgaccatgtctgaaatgtcagttctctagcataatttgtattgaaatgaaaccaccagtgttatcaacttgaatgtaaatgtacatgtgcagatattcctaaagttttattgacaaaaaaaaaaaaaaaaa

SEQ ID NO: 37 - DNA, 786 bp, Homo sapiens
acagaaggacgaaccagtgagctaagctgcggggcgcgggctcggccggggcaccggtgagtcgccggcgctgcagagggaggcggcactggtctcgacgtggggcggccagcgatgaagccgcccagttcaatacaaacaagttgtaaatttaaagatgttagaagaaatgtccaaaaagatacagaagaactaaagagctgtggtatacaagacatatttgttttctgcaccagaggggaactgtcaaaatatagagtcccaaaccttctggatctctaccagcaatgtggaattatcacccatcatcatccaatcgcagatggagggactcctgacatagccagctgctgtgaaataatggaagagcttacaacctgccttaaaaattaccgaaaaaccttaatacactgctatggaggacttgggagatcttgtcttgtagctgcttgtctcctactatacctgtctgacacaatatcaccagagcaagccatagacagcctgcgagacctaagaggatccggggcaatacagaccatcaagcaatacaattatcttcatgagtttcgggacaaattagctgcacatctatcatcaagagattcacaatcaagatctgtatcaagataaaggaattcaaatagcatatatatgaccatgtctgaaatgtcagttctctagcataatttgtattgaaatgaaaccaccagtgttatcaacttgaatgtaaatgtacatgtgcagatattcctaaagttttattgacaaaaaaaaaaaaaaaaa

SEQ ID NO: 38 - DNA, 4786 bp, Homo sapiens
ctcggcgctgaaattcaaatttgaacggctgcagaggccgagtccgtcactggaagccgagaggagaggacagctggttgtgggagagttcccccgcctcagactcctggttttttccaggagacacactgagctgagactcacttttctcttcctgaatttgaaccaccgtttccatcgtctcgtagtccgacgcctggggcgatggatccgtttacggagaaactgctggagcgaacccgtgccaggcgagagaatcttcagagaaaaatggctgagaggcccacagcagctccaaggtctatgactcatgctaagcgagctagacagccactttcagaagcaagtaaccagcagcccctctctggtggtgaagagaaatcttgtacaaaaccatcgccatcaaaaaaacgctgttctgacaacactgaagtagaagtttctaacttggaaaataaacaaccagttgagtcgacatctgcaaaatcttgttctccaagtcctgtgtctcctcaggtgcagccacaagcagcagataccatcagtgattctgttgctgtcccggcatcactgctgggcatgaggagagggctgaactcaagattggaagcaactgcagcctcctcagttaaaacacgtatgcaaaaacttgcagagcaacggcgccgttgggataatgatgatatgacagatgacattcctgaaagctcactcttctcaccaatgccatcagaggaaaaggctgcttcccctcccagacctctgctttcaaatgcctcggcaactccagttggcagaaggggccgtctggccaatcttgctgcaactatttgctcctgggaagatgatgtaaatcactcatttgcaaaacaaaacagtgtacaagaacagcctggtaccgcttgtttatccaaattttcctctgcaagtggagcatctgctaggatcaatagcagcagtgttaagcaggaagctacattctgttcccaaagggatggcgatgcctctttgaataaagccctatcctcaagtgctgatgatgcgtctttggttaatgcctcaatttccagctctgtgaaagctacttctccagtgaaatctactacatctatcactgatgctaaaagttgtgagggacaaaatcctgagctacttccaaaaactcctattagtcctctgaaaacgggggtatcgaaaccaattgtgaagtcaactttatcccagacagttccatccaagggagaattaagtagagaaatttgtctgcaatctcaatctaaagacaaatctacgacaccaggaggaacaggaattaagcctttcctggaacgctttggagagcgttgtcaagaacatagcaaagaaagtccagctcgtagcacaccccacagaacccccattattactccaaatacaaaggccatccaagaaagattattcaagcaagacacatcttcatctactacccatttagcacaacagctcaagcaggaacgtcaaaaagaactagcatgtcttcgtggccgatttgacaagggcaatatatggagtgcagaaaaaggcggaaactcaaaaagcaaacaactagaaaccaaacaggaaactcactgtcagagcactcccctcaaaaaacaccaaggtgtttcaaaaactcagtcacttccagtaacagaaaaggtgaccgaaaaccagataccagccaaaaattctagtacagaacctaaaggtttcactgaatgcgaaatgacgaaatctagccctttgaaaataacattgtttttagaagaggacaaatccttaaaagtaacatcagacccaaaggttgagcagaaaattgaagtgatacgtgaaattgagatgagtgtggatgatgatgatatcaatagttcgaaagtaattaatgacctcttcagtgatgtcctagaggaaggtgaactagatatggagaagagccaagaggagatggatcaagcattagcagaaagcagcgaagaacaggaagatgcactgaatatctcctcaatgtctttacttgcaccattggcacaaacagttggtgtggtaagtccagagagtttagtgtccacacctagactggaattgaaagacaccagcagaagtgatgaaagtccaaaaccaggaaaattccaaagaactcgtgtccctcgagctgaatctggtgatagccttggttctgaagatcgtgatcttctttacagcattgatgcatatagatctcaaagattcaaagaaacagaacgtccatcaataaagcaggtgattgttcggaaggaagatgttacttcaaaactggatgaaaaaaataatgcctttccttgtcaagttaatatcaaacagaaaatgcaggaactcaataacgaaataaatatgcaacagacagtgatctatcaagctagccaggctcttaactgctgtgttgatgaagaacatggaaaagggtccctagaagaagctgaagcagaaagacttcttctaattgcaactgggaagagaacacttttgattgatgaattgaataaattgaagaacgaaggacctcagaggaagaataaggctagtccccaaagtgaatttatgccatccaaaggatcagttactttgtcagaaatccgcttgcctctaaaagcagattttgtctgcagtacggttcagaaaccagatgcagcaaattactattacttaattatactaaaagcaggagctgaaaatatggtagccacaccattagcaagtacttcaaactctcttaacggtgatgctctgacattcactactacatttactctgcaagatgtatccaatgactttgaaataaatattgaagtttacagcttggtgcaaaagaaagatccctcaggccttgataagaagaaaaaaacatccaagtccaaggctattactccaaagcgactcctcacatctataaccacaaaaagcaacattcattcttcagtcatggccagtccaggaggtcttagtgctgtgcgaaccagcaacttcgcccttgttggatcttacacattatcattgtcttcagtaggaaatactaagtttgttctggacaaggtcccctttttatcttctttggaaggtcatatttatttaaaaataaaatgtcaagtgaattccagtgttgaagaaagaggttttctaaccatatttgaagatgttagtggttttggtgcctggcatcgaagatggtgtgttctttctggaaactgtatatcttattggacttatccagatgatgagaaacgcaagaatcccataggaaggataaatctggctaattgtaccagtcgtcagatagaaccagccaacagagaattttgtgcaagacgcaacacttttgaattaattactgtccgaccacaaagagaagatgaccgagagactcttgtcagccaatgcagggacacactctgtgttaccaagaactggctgtctgcagatactaaagaagagcgggatctctggatgcaaaaactcaatcaagttcttgttgatattcgcctctggcaacctgatgcttgctacaaacctattggaaagccttaaaccgggaaatttccatgctatctagaggtttttgatgtcatcttaagaaacacacttaagagcatcagatttactgattgcattttatgctttaagtacgaaagggtttgtgccaatattcactacgtattatgcagtatttatatcttttgtatgtaaaactttaactgatttctgtcattcatcaatgagtagaagtaaatacattatagttgattttgctaaatcttaatttaaaagcctcattttcctagaaatctaattattcagttattcatgacaatatttttttaaaagtaagaaattctgagttgtcttcttggagctgtaggtcttgaagcagcaacgtctttcaggggttggagacagaaacccattctccaatctcagtagttttttcgaaaggctgtgatcatttattgatcgtgatatgacttgttactagggtactgaaaaaaatgtctaaggcctttacagaaacatttttagtaatgaggatgagaactttttcaaatagcaaatatatattggcttaaagcatgaggctgtcttcagaaaagtgatgtggacataggaggcaatgtgtgagacttgggggttcaatattttatatagaagagttaataagcacatggtttacatttactcagctactatatatgcagtgtggtgcacattttcacagaattctggcttcattaagatcattatttttgctgcgtagcttacagacttagcatattagttttttctactcctacaagtgtaaattgaaaaatctttatattaaaaaagtaaactgttatgaagctgctatgtactaataatactttgcttgccaaagtgtttgggttttgttgttgtttgtttgtttgtttgtttttggttcatgaacaacagtgtctagaaacccattttgaaagtggaaaattattaagtcacctatcacctttaaacgcctttttttaaaattataaaatattgtaaagcagggtctcaacttttaaatacactttgaacttcttctctgaattattaaagttctttatgacctcatttataaacactaaattctgtcacctcctgtcattttattttttattcattcaaatgtattttttcttgtgcatattataaaaatatattttatgagctcttactcaaataaatacctgtaaatgtctaaaggaaaaaaaaaaaaaaaaaa

SEQ ID NO: 39 - DNA, 1659 bp, Homo sapiens
agtgcgcctgcgcggagctcgtggccgcgcctgctcccgccgggggctccttgctcggccgggccgcggccatgggagaggccgaggtgggcggcgggggcgccgcaggcgacaagggcccgggggaggcggccaccagcccggcggaggagacagtggtgtggagccccgaggtggaggtgtgcctcttccacgccatgctgggccacaagcccgtcggtgtgaaccgacacttccacatgatttgtattcgggacaagttcagccagaacatcgggcggcaggtcccatccaaggtcatctgggaccatctgagcaccatgtacgacatgcaggcgctgcatgagtctgagattcttccattcccgaatccagagaggaacttcgtccttccagaagagatcattcaggaggtccgagaaggaaaagtgatgatagaagaggagatgaaagaggagatgaaggaagacgtggacccccacaatggggctgacgatgttttttcatcttcagggagtttggggaaagcatcagaaaaatccagcaaagacaaagagaagaactcctcagacttggggtgcaaagaaggcgcagacaagcggaagcgcagccgggtcaccgacaaagtcctgaccgcaaacagcaacccttccagtcccagtgctgccaagcggcgccgcacgtagaccctcagccctggtggcggcagagaagcgggcgaggcactgtggtcgctgagggggttggctgggtctgagtgccaccccccaggccacagtgataccatcccagtgccatgagcccacactgcccgccctcaggctctcaggtgaacgtggccgtcagcggggaaacgtgtgtgtcagttggaccatgtgggaccctgatggacctgaaagaccaggatcggtccagctcagatattgagggctctgaagcctagttctgtcttctctggagcagctgtggcttccccgtggctgcttggtgacatggattagcgctacgtgggctgcagcatttgggatccaggctacctagaggggcatcgggccagggaaaacctcggattagcaagcaataaaaacatgacctcactcttcctcaaaggagcccctggtcttccctgtgtgactcagttctttccatctgtttgtcccgctgcaagcctctttctgcgctgactgtgacattggaacgtggccttcctgtcaccccctccgtgccacgcactgaaggccacccccacccacctgggaaactaagaactggatattttgcctcattcacttgtactgtaacaatgtatataatttggttggtatttcactatttaatttttaagaagcctattttactagtgttttatatgaacaaagtactgcagaagttaaacctgtgttgtattttttctgagatgttttgctttaagagatactttttgctcagtttttatatgccagatacagagaatttgtagcggttatttttgtatgatctagtaacttgcaaacagaccaaatggatgagaggcggggaccgtgcagctgtcggctgatgaggaggcggccgccccagtgctgatggagatgccactttcgtgtgactgcgaacattaaagcacaaaaaaatccaaaaaaaaaaaaaaaaaaaaaaaaa

SEQ ID NO: 40 - DNA, 600 bp, Homo sapiens
agtcttggcggaggtgaccaaagccacgtaatgtccgtagttcgctcatccgtccatgccagatggattgtggggaaggtgattgggacaaaaatgcaaaagactgctaaagtgagagtgaccaggcttgttctggatccctatttattaaagtattttaataagcggaaaacctactttgctcacgatgcccttcagcagtgcacagttggggatattgtgcttctcagagctttacctgttccacgagcaaagcatgtgaaacatgaactggctgagatcgttttcaaagttggaaaagtcatagatccagtgacaggaaagccctgtgctggaactacctacctggagagtccgttgagttcggaaaccacccagctaagcaaaaatctggaagaactcaatatctcttcagcacagtgaagcgggagtggaagaaggatctaaagggaaaaactgacatgtttatgttatggaaaaagaaatttttctaagtttcatcacaaactgtgtccagtttctctgtggtgtttatgaaatagctaaaagcaaatgaagtaaagggcatactatggtttttcacaaaaaaaaaaaaaaaaa

SEQ ID NO: 41 - DNA, 6595 bp, Homo sapiens
gctccctgggctgctggtcttctttaccttccagctgctcacagaacagagagtttctacatacaagcagaagatgtgaaaatattgggaataaataaagtatatgcttataaacaagttgtggctgctcctggttacgttgtgcctgaccgaggaactggcagcagcgggagagaagtcttatggaaagccatgtgggggccaggactgcagtgggagctgtcagtgttttcctgagaaaggagcgagaggacgacctggaccaattggaattcaaggcccaacaggtcctcaaggattcactggctctactggtttatcgggattgaaaggagaaaggggtttcccaggccttctgggaccttatggaccaaaaggagataagggtcccatgggagttcctggctttcttggcatcaatgggattccgggccaccctggacaaccaggccccagaggcccacctggtctggatggctgtaatggaactcaaggagctgttggatttccaggccctgatggctatcctgggcttctcggaccacccgggcttcctggtcagaaaggatcaaaaggtgaccctgtccttgctccaggtagtttcaaaggaatgaagggggatcctgggctgcctggactggatggaatcactggcccacaaggagcacccggatttcctggagctgtaggacctgcaggaccaccaggattacaaggtcctccagggcctcctggtcctcttggtcctgatgggaatatggggctaggttttcaaggagagaaaggagtcaagggggatgttggcctccctggcccagcaggacctccaccatctactggagagctggaattcatgggattccccaaagggaagaaaggatccaagggtgaaccagggcctaagggttttccaggcataagtggccctccaggcttcccgggccttggaactactggagaaaagggagaaaagggagaaaagggaatccctggtttgccaggacctaggggtcccatgggttcagaaggagtccaaggccctccagggcaacagggcaagaaagggaccctgggatttcctgggcttaatggattccaaggaattgagggtcaaaagggtgacattggcctgccaggcccagatgttttcatcgatatagatggtgctgtgatctcaggtaatcctggagatcctggtgtacctggcctcccaggccttaaaggagatgaaggcatccaaggcctacgtggcccttctggtgtccctggattgccagcattatcaggtgtcccaggagccctagggcctcagggatttccagggctgaagggggaccaaggaaacccaggccgtaccacaattggagcagctggcctccctggcagagatggtttgccaggcccaccaggtccaccaggcccacctagtccagaatttgagactgaaactctacacaacaaagagtcagggttccctggtctccgaggagaacaaggtccaaaaggaaacctaggcctcaaaggaataaaaggagactcaggtttctgtgcttgtgacggtggtgttcccaacactggaccacccggggaaccaggcccacctggtccatggggtctcataggccttccaggccttaaaggagccagaggagatcgaggctctgggggtgcacagggcccagcaggggctccaggcttagttgggcctctgggtccttcaggacccaaaggaaagaagggggaaccaattctcagtacaatccaaggaatgccaggagatcggggtgattctggctcccagggcttccgtggtgtaataggagaaccaggcaaggacggagtaccaggtttaccaggtctgccaggccttccgggtgatggtggacagggcttcccaggtgaaaaggggttacctggacttcctggtgaaaaaggccatcctggtccacctggcctcccaggaaatgggttaccaggacttcctggaccccgtgggcttcctggagataaaggcaaggatggattaccgggacaacaaggccttcccggatctaagggaatcaccctgccctgtattattcctgggtcatacggtccatcaggatttccaggcactcccggattcccaggccctaaagggtctcgaggcctccctgggaccccaggccagcctgggtcaagtggaagtaaaggagagccagggagtccaggattggttcatcttcctgaattaccaggatttcctggacctcgtggggagaagggcttgcctgggtttcctgggctccctggaaaagatggcttgcctgggatgattggcagtccaggcttacctggttccaagggagccactggtgacatctttggtgctgaaaatggtgctccgggggaacaaggcctacaaggattaacagggcacaaaggatttcttggagactctggccttccaggactcaagggtgtgcacgggaagcctggcttactaggccccaaaggtgagcggggcagccctgggacaccaggacaggtgggacagccaggcaccccaggatctagtggtccatatggcatcaagggcaaatctgggctcccaggagcaccaggcttcccaggcatctcaggacatcctggaaagaaaggaacaagaggcaagaaaggtcctcctggatcaattgtaaagaaagggctgccagggctaaaaggccttcctggaaatccaggcctagtaggactgaaaggaagcccaggctctccaggggtcgctgggttgccagccctctctggacccaagggagagaaggggtctgttggattcgtaggttttccaggaataccaggtctgcctggtattcctggaacaagaggattaaaaggaattccaggatcaactggaaaaatgggaccatctggacgtgctggtactcctggtgaaaagggagacagaggcaatccggggccagtcggaatacctagtccaagacgtccaatgtcaaacctttggctcaaaggagacaaaggctctcaaggctcagccggatccaatggatttcctgggccaagaggtgacaaaggagaggctggtcgacctggaccaccaggcctacctggagctcctggcctcccaggcattatcaaaggagttagtggaaagccagggccccctggcttcatgggaatccggggcttacctggcctgaaggggtcctctgggatcacaggtttcccaggaatgccaggagaaagtggttcacaaggtatcagagggtcgcctggactcccaggagcatctggtctcccaggcctgaaaggagacaacggccagacagttgaaatttccggtagcccaggacccaagggacagcctggcgaatctggttttaaaggcacaaaaggaagagatggactaataggcaatataggcttccctggaaacaaaggtgaagatggaaaagttggtgtttctggagatgttggccttcctggagctccaggatttccaggagttgccggcatgagaggagaaccaggacttccaggttcttctggtcaccaaggggcaattgggcctctaggatcccccggattaataggacccaaaggcttccctggatttcctggtttacatggactgaatgggcttccgggcaccaagggtacccatggcactccaggacctagtatcaccggtgtgcctgggcctgctggtctccctggacccaaaggagaaaaaggatatccaggaattggcatcggagctccagggaagccgggcctgagagggcaaaaaggtgatcgaggtttcccaggtctccagggccctgctggtctccccggtgccccaggcatctccttgccctcactcatagcaggacagcctggtgaccccgggcgaccaggcctagatggagaacgaggccgcccaggccccgctggacccccaggtccccctgggccatcctcgaatcaaggcgacaccggagaccctggcttccctggaattcctggacctaaagggcctaagggagaccaaggaattccaggtttttctggcctccctggagagctaggactgaaaggcatgagaggtgagcctggcttcatggggactccaggcaaggttgggccacctggagacccaggatttcccggaatgaaggggaaggcagggccaagaggctcttctggcctccaaggtgatcctggacaaacaccaactgcagaagctgtccaggttcctcctggacccttgggtctaccagggatcgatggcatccctggcctcactggggaccctggggctcaaggccctgtaggcctacaaggctccaaaggtttacctggcatccccggtaaagatggccccagtgggctcccaggcccacctggggctcttggtgatcctggtctgcctggactgcaaggccctccaggatttgaaggagctccagggcagcaaggccccttcgggatgcctggaatgcctggccagagcatgagagtgggctacacgttggtaaagcacagccagtcggaacaggtgcccccgtgtcccatcgggatgagccagctgtgggtggggtacagcttactgtttgtggaggggcaagagaaagcccacaaccaggacctgggctttgctggctcctgtctgccccgcttcagcaccatgcccttcatctactgcaacatcaacgaggtgtgccactatgccaggcgcaatgataaatcttactggctctccactaccgcccctatccccatgatgcccgtcagccagacccagattccccagtacatcagccgctgctctgtgtgtgaggcaccctcgcaagccattgctgtgcacagccaggacatcaccatcccgcagtgccccctgggctggcgcagcctctggattgggtactctttcctcatgcacactgccgctggtgccgagggtggaggccagtccctggtctcacctggctcctgcctagaggactttcgggccactcctttcatcgaatgcagtggtgcccgaggcacctgccactactttgcaaacaagtacagtttctggttgaccacagtggaggagaggcagcagtttggggagttgcctgtgtctgaaacgctgaaagctgggcagctccacactcgagtcagtcgctgccaggtgtgtatgaaaagcctgtagggtggcacctgccactctgccccttgccctcccctgcccctcacaacagtcacctcacaaacctgaatggtctgaagaaggaaggcctgagcccctttgcctgtcaagttgtacattggagtctcatttgggctagactaccggacactcgtcaccccagccctcgggtccatagagatgagcccaccctgctgagatctgctgtcctgtttctgtcaagctggtgctactgtttgatttggatgattgtgtgactattcatggctacctcagaaagatttgatgggccacaactgtcttagactgctagctttctccttaccgtcttgatcggaaagctcttcctaatcgctaatcagtcatttcttcatgtacagaggtcagcacacattatttggcttaaaccagaacccagtgtttccacacttaaattctctaaccgaatattcatggatggctcaagtctgcacagagcaagtcctcactcttcaaggaggcccactgtgtctaggcaggcaagagaattgaaatgaggtgccacccagtagcccagagtgagctttagctctctctagaatgagcaagactgggccccacatggcttagagaggcttgaaggccagcagctgggttgggggtggtggtcattaatggcatatggtcctagacaaaccatctcctccttgccggctccccctccagccagagacagaggatgtggcctggttcaaagtaaagcagaggatgcaacaaatgtggccaagcctatcaaaggaaatgagaatgacagccttttttcctgggccagaagtagaggggtgggtgcgtaaggatgtgtgagttttgcttttgactccaggaacaaaaaggtaaatcccacatcccagtttctcagaagtccctgtttattccaaatgccatccagatgtgtgcaatgtggcaaactgaagctgcacagtgttggtttccttgtattctgaggatgttaaagactttgttaaatggttatccaattgctctttcacaggtagcctattaaactattttaatatgtttttttaaacctcataaaaatctagcacactcttctcttgagcagttagcagacctaaagcaagcctgaattggctatgcagtacattgtattctgtttgggggaatttgttttagccattttctttaattaccagttttccagaacactcttagctatgttgacatgaggcagttccttccaggtgattctgtttccttaagtattatataaactgtgccaatacagacaaagcataatcaatataatctgaattattgttatctttacctcctgagtaataagcatggtgtcagttttgtacatagcaaataaaataaatgaaatctgaacatgtgaaaaaaaaaaaaaaaaaaaaaaaa

SEQ ID NO: 42 - DNA, 6721 bp, Homo sapiens
ggaactatctcctgagtgctgcaagttgtaacgggcaccgctgagcctgtttccctttggagcacttcttatctagaagcagtgtttagtttcttccaaactgggccacttcgtccacctactctgttctgagtaaggaaacagcctccaagcatcagcagagcccagatgagcacgggccgcggagccgcttagcagtctcccgggacccagctccggaggagccgcaagcatgcaccctgggttgtggctgctcctggttacgttgtgcctgaccgaggaactggcagcagcgggagagaagtcttatggaaagccatgtgggggccaggactgcagtgggagctgtcagtgttttcctgagaaaggagcgagaggacgacctggaccaattggaattcaaggcccaacaggtcctcaaggattcactggctctactggtttatcgggattgaaaggagaaaggggtttcccaggccttctgggaccttatggaccaaaaggagataagggtcccatgggagttcctggctttcttggcatcaatgggattccgggccaccctggacaaccaggccccagaggcccacctggtctggatggctgtaatggaactcaaggagctgttggatttccaggccctgatggctatcctgggcttctcggaccacccgggcttcctggtcagaaaggatcaaaaggtgaccctgtccttgctccaggtagtttcaaaggaatgaagggggatcctgggctgcctggactggatggaatcactggcccacaaggagcacccggatttcctggagctgtaggacctgcaggaccaccaggattacaaggtcctccagggcctcctggtcctcttggtcctgatgggaatatggggctaggttttcaaggagagaaaggagtcaagggggatgttggcctccctggcccagcaggacctccaccatctactggagagctggaattcatgggattccccaaagggaagaaaggatccaagggtgaaccagggcctaagggttttccaggcataagtggccctccaggcttcccgggccttggaactactggagaaaagggagaaaagggagaaaagggaatccctggtttgccaggacctaggggtcccatgggttcagaaggagtccaaggccctccagggcaacagggcaagaaagggaccctgggatttcctgggcttaatggattccaaggaattgagggtcaaaagggtgacattggcctgccaggcccagatgttttcatcgatatagatggtgctgtgatctcaggtaatcctggagatcctggtgtacctggcctcccaggccttaaaggagatgaaggcatccaaggcctacgtggcccttctggtgtccctggattgccagcattatcaggtgtcccaggagccctagggcctcagggatttccagggctgaagggggaccaaggaaacccaggccgtaccacaattggagcagctggcctccctggcagagatggtttgccaggcccaccaggtccaccaggcccacctagtccagaatttgagactgaaactctacacaacaaagagtcagggttccctggtctccgaggagaacaaggtccaaaaggaaacctaggcctcaaaggaataaaaggagactcaggtttctgtgcttgtgacggtggtgttcccaacactggaccacccggggaaccaggcccacctggtccatggggtctcataggccttccaggccttaaaggagccagaggagatcgaggctctgggggtgcacagggcccagcaggggctccaggcttagttgggcctctgggtccttcaggacccaaaggaaagaagggggaaccaattctcagtacaatccaaggaatgccaggagatcggggtgattctggctcccagggcttccgtggtgtaataggagaaccaggcaaggacggagtaccaggtttaccaggtctgccaggccttccgggtgatggtggacagggcttcccaggtgaaaaggggttacctggacttcctggtgaaaaaggccatcctggtccacctggcctcccaggaaatgggttaccaggacttcctggaccccgtgggcttcctggagataaaggcaaggatggattaccgggacaacaaggccttcccggatctaagggaatcaccctgccctgtattattcctgggtcatacggtccatcaggatttccaggcactcccggattcccaggccctaaagggtctcgaggcctccctgggaccccaggccagcctgggtcaagtggaagtaaaggagagccagggagtccaggattggttcatcttcctgaattaccaggatttcctggacctcgtggggagaagggcttgcctgggtttcctgggctccctggaaaagatggcttgcctgggatgattggcagtccaggcttacctggttccaagggagccactggtgacatctttggtgctgaaaatggtgctccgggggaacaaggcctacaaggattaacagggcacaaaggatttcttggagactctggccttccaggactcaagggtgtgcacgggaagcctggcttactaggccccaaaggtgagcggggcagccctgggacaccaggacaggtgggacagccaggcaccccaggatctagtggtccatatggcatcaagggcaaatctgggctcccaggagcaccaggcttcccaggcatctcaggacatcctggaaagaaaggaacaagaggcaagaaaggtcctcctggatcaattgtaaagaaagggctgccagggctaaaaggccttcctggaaatccaggcctagtaggactgaaaggaagcccaggctctccaggggtcgctgggttgccagccctctctggacccaagggagagaaggggtctgttggattcgtaggttttccaggaataccaggtctgcctggtattcctggaacaagaggattaaaaggaattccaggatcaactggaaaaatgggaccatctggacgtgctggtactcctggtgaaaagggagacagaggcaatccggggccagtcggaatacctagtccaagacgtccaatgtcaaacctttggctcaaaggagacaaaggctctcaaggctcagccggatccaatggatttcctgggccaagaggtgacaaaggagaggctggtcgacctggaccaccaggcctacctggagctcctggcctcccaggcattatcaaaggagttagtggaaagccagggccccctggcttcatgggaatccggggcttacctggcctgaaggggtcctctgggatcacaggtttcccaggaatgccaggagaaagtggttcacaaggtatcagagggtcgcctggactcccaggagcatctggtctcccaggcctgaaaggagacaacggccagacagttgaaatttccggtagcccaggacccaagggacagcctggcgaatctggttttaaaggcacaaaaggaagagatggactaataggcaatataggcttccctggaaacaaaggtgaagatggaaaagttggtgtttctggagatgttggccttcctggagctccaggatttccaggagttgccggcatgagaggagaaccaggacttccaggttcttctggtcaccaaggggcaattgggcctctaggatcccccggattaataggacccaaaggcttccctggatttcctggtttacatggactgaatgggcttccgggcaccaagggtacccatggcactccaggacctagtatcaccggtgtgcctgggcctgctggtctccctggacccaaaggagaaaaaggatatccaggaattggcatcggagctccagggaagccgggcctgagagggcaaaaaggtgatcgaggtttcccaggtctccagggccctgctggtctccccggtgccccaggcatctccttgccctcactcatagcaggacagcctggtgaccccgggcgaccaggcctagatggagaacgaggccgcccaggccccgctggacccccaggtccccctgggccatcctcgaatcaaggcgacaccggagaccctggcttccctggaattcctggacctaaagggcctaagggagaccaaggaattccaggtttttctggcctccctggagagctaggactgaaaggcatgagaggtgagcctggcttcatggggactccaggcaaggttgggccacctggagacccaggatttcccggaatgaaggggaaggcagggccaagaggctcttctggcctccaaggtgatcctggacaaacaccaactgcagaagctgtccaggttcctcctggacccttgggtctaccagggatcgatggcatccctggcctcactggggaccctggggctcaaggccctgtaggcctacaaggctccaaaggtttacctggcatccccggtaaagatggccccagtgggctcccaggcccacctggggctcttggtgatcctggtctgcctggactgcaaggccctccaggatttgaaggagctccagggcagcaaggccccttcgggatgcctggaatgcctggccagagcatgagagtgggctacacgttggtaaagcacagccagtcggaacaggtgcccccgtgtcccatcgggatgagccagctgtgggtggggtacagcttactgtttgtggaggggcaagagaaagcccacaaccaggacctgggctttgctggctcctgtctgccccgcttcagcaccatgcccttcatctactgcaacatcaacgaggtgtgccactatgccaggcgcaatgataaatcttactggctctccactaccgcccctatccccatgatgcccgtcagccagacccagattccccagtacatcagccgctgctctgtgtgtgaggcaccctcgcaagccattgctgtgcacagccaggacatcaccatcccgcagtgccccctgggctggcgcagcctctggattgggtactctttcctcatgcacactgccgctggtgccgagggtggaggccagtccctggtctcacctggctcctgcctagaggactttcgggccactcctttcatcgaatgcagtggtgcccgaggcacctgccactactttgcaaacaagtacagtttctggttgaccacagtggaggagaggcagcagtttggggagttgcctgtgtctgaaacgctgaaagctgggcagctccacactcgagtcagtcgctgccaggtgtgtatgaaaagcctgtagggtggcacctgccactctgccccttgccctcccctgcccctcacaacagtcacctcacaaacctgaatggtctgaagaaggaaggcctgagcccctttgcctgtcaagttgtacattggagtctcatttgggctagactaccggacactcgtcaccccagccctcgggtccatagagatgagcccaccctgctgagatctgctgtcctgtttctgtcaagctggtgctactgtttgatttggatgattgtgtgactattcatggctacctcagaaagatttgatgggccacaactgtcttagactgctagctttctccttaccgtcttgatcggaaagctcttcctaatcgctaatcagtcatttcttcatgtacagaggtcagcacacattatttggcttaaaccagaacccagtgtttccacacttaaattctctaaccgaatattcatggatggctcaagtctgcacagagcaagtcctcactcttcaaggaggcccactgtgtctaggcaggcaagagaattgaaatgaggtgccacccagtagcccagagtgagctttagctctctctagaatgagcaagactgggccccacatggcttagagaggcttgaaggccagcagctgggttgggggtggtggtcattaatggcatatggtcctagacaaaccatctcctccttgccggctccccctccagccagagacagaggatgtggcctggttcaaagtaaagcagaggatgcaacaaatgtggccaagcctatcaaaggaaatgagaatgacagccttttttcctgggccagaagtagaggggtgggtgcgtaaggatgtgtgagttttgcttttgactccaggaacaaaaaggtaaatcccacatcccagtttctcagaagtccctgtttattccaaatgccatccagatgtgtgcaatgtggcaaactgaagctgcacagtgttggtttccttgtattctgaggatgttaaagactttgttaaatggttatccaattgctctttcacaggtagcctattaaactattttaatatgtttttttaaacctcataaaaatctagcacactcttctcttgagcagttagcagacctaaagcaagcctgaattggctatgcagtacattgtattctgtttgggggaatttgttttagccattttctttaattaccagttttccagaacactcttagctatgttgacatgaggcagttccttccaggtgattctgtttccttaagtattatataaactgtgccaatacagacaaagcataatcaatataatctgaattattgttatctttacctcctgagtaataagcatggtgtcagttttgtacatagcaaataaaataaatgaaatctgaacatgtgaaaaaaaaaaaaaaaaaaaaaaaa

SEQ ID NO: 43 - DNA, 2860 bp, Homo sapiens
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgaggcgagccaccatttcaaacccaataacaggagacttggagacggtacattacagaattagcaaaagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 44 - DNA, 2953 bp, Homo sapiens
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccagagatgaagtcttgctatgttgcccggcctggtctcaaactcctgagctcaagtgatcctctttccttggcctcccaaagtactgggattacaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgaggcgagccaccatttcaaacccaataacaggagacttggagacggtacattacagaattagcaaaagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 45 - DNA, 2806 bp, Homo sapiens
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacagaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 46 - DNA, 2860 bp, Homo sapiens
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 47 - DNA, 1432 bp, Homo sapiens
ggggcgtgctcgcggctataaggggcggaggctgggcggcgttgctctgcgctctgcggctgacggcgcttttgtctccggtgagttttgtggcgggaagcttctgcgctggtgcttagtaaccgactttcctccggactcctgcacgacctgctcctacagccggcgatccactcccggctgttcccccggagggtccagaggcctttcagaaggagaaggcagctctgtttctctgcagaggagtagggtcctttcagccatgaagcatgtgttgaacctctacctgttaggtgtggtactgaccctactctccatcttcgttagagtgatggagtccctagagggcttactagagagcccatcgcctgggacctcctggaccaccagaagccaactagccaacacagagcccaccaagggccttccagaccatccatccagaagcatgtgataagacctccttccatactggccatattttggaacactgacctagacatgtccagatgggagtcccattcctagcagacaagctgagcaccgttgtaaccagagaactattactaggccttgaagaacctgtctaactggatgctcattgcctgggcaaggcctgtttaggccggttgcggtggctcatgcctgtaatcctagcactttgggaggctgaggtgggtggatcacctgaggtcaggagttcgagaccagcctcgccaacatggcgaaaccccatctctactaaaaatacaaaagttagctgggtgtggtggcagaggcctgtaatcccagctccttgggaggctgaggcgggagaattgcttgaacccggggacggaggttgcagtgagccgagatcgcactgctgtacccagcctgggccacagtgcaagactccatctcaaaaaaaaaagaaaagaaaaagcctgtttaatgcacaggtgtgagtggattgcttatggctatgagataggttgatctcgcccttaccccggggtctggtgtatgctgtgctttcctcagcagtatggctctgacatctcttagatgtcccaacttcagctgttgggagatggtgatattttcaaccctacttcctaaacatctgtctggggttcctttagtcttgaatgtcttatgctcaattatttggtgttgagcctctcttccacaagagctcctccatgtttggatagcagttgaagaggttgtgtgggtgggctgttgggagtgaggatggagtgttcagtgcccatttctcattttacattttaaagtcgttcctccaacatagtgtgtattggtctgaagggggtggtgggatgccaaagcctgctcaagttatggacattgtggccaccatgtggcttaaatgattttttctaactaataaagtggaatatatatttctaaaaaaaaaaaaaaaaaaaaa

SEQ ID NO: 48 - DNA, 3081 bp, Homo sapiens
attagaggctccagccccgccgacttgcagacgtgagatcgggcacacctgagcggcggcggggcggtcgtggccacatccggggcgacgtgcctgagtcaccccgtcccgccagcgtctgccagtccagccagtccgcccagtctctcgcgtccgagactcgcctccagcctcccacctccgcccgggccgcgcgagcctcgcgggggcgggggcggggcgccaaggggcggggctgtctcttaaagggccccgggccgctgcccttaggccacttcctgggggcggagaggacctcagcggctgcggcgacacccagggaaggcggcgcggccgggtcccgaaactcctggctgtttccatcagagccctcggacactcccagcccgggctgagcacgcatcgtcgctccccggcggatacaagggggctccgccatccgctcccgtcagttcggcctccatctcctgggacccgcgccggcagccaggccaggcctctgagtggccccagagccctggctggactcgtccacggcggcagcgatctgcccggggtctcggaggccatcccttcagagtcggccctgtgctcgccaccgtcacctgctggttggattccggaaacccactgtctgaagaccacagaggggtgtcgctgaccaccccaaatcggatacgtccagacctcaagctcccttcccctctctggctgccctctgctcttttcatctcttctctcaaccttttggggatttctgtgtcctgacaccacctccccatccaccaccaaagtagccggggtgagccccaaaccttactgggtgtgctccacctgtgcctccaacccagcgaatctgacagcttcgacccaattctgcacacacccaggaagttctgccttttcttttctttcggtgtctcctgtacttcccaaaatttctcctcctcctgtgccctcttcgcccccctcctttgggggccccgtgaccctgaatgtggggggcacactatattccaccactttggagaccctgacccgcttcccagactctatgctgggggccatgtttagggccggcacccccatgccccccaacctcaattcccaaggaggcggccactacttcatcgaccgggatggcaaggccttccggcacatcctcaatttcctgaggctgggccgcctggacctgccccgtgggtacggagagacagcgctgctcagggcagaggctgacttctaccagatccggcccctcctggacgcgctgcgggaactggaggcctctcaggggacccctgcacccacagctgccctgctccacgcagatgtagatgtcagcccccgcctggtgcacttctctgctcgccggggaccccatcactatgagctgagctccgtccaggtggacaccttccgagccaaccttttctgcaccgactctgagtgtctaggtgctttgcgggcccgatttggtgtggccagtggggatagggcagaggggagcccacattttcatctggagtgggccccccgccccgtggaactccccgaggtggagtatgggagactggggctgcagccgctgtggactggggggccaggagagcggcgggaggtggtgggcaccccaagcttcctggaggaggtgctgcgggtggctctcgagcacggcttccgactagactctgtcttccccgaccccgaagacctgctcaactccaggtctctgcgctttgtccggcactgaggatgctgttctcagtttgactgtggggaggagagagaatggggtactagcacccctgaagcctctttccagctctgcttcaggagctatgagagtcgggactctcctgcacctgactggagctcagatgtgggcaggaattcccaaacctgagcccaccaaggactcacaagtggtccagaaggtctcaacctgtgctgaccctgggaggggtagggaaggttctctcagcttgttcttgcctaaggctgagcacctccagtctctccttgatttggagctcagtgtttaagggcttggaaaaggggggaacatctctttacccagactagacctagcaaaaccctggaaggatattgaggtctggggaaaagggaggactttgcattttcccaatgcggtctcttggaccatggcttctactcctgaagctgggtggcctggcctggcctgaccaatgagaggccagaacactctggaacatcggaagaggagttctttgctatgttccaagccatctactgagggaggcagaaaggccacaacccaccctaggttgatgtatgggagctaggacagtccccatggcaatggggctggagcatccctcatctggaagaatcccatactgatggcagggctggccagggggaagagggtagtatctgtgggtcctggcctttcttcatgtgtgcgtgcatatcagcccgtgtggctgactgatgtataggtccctggcatcctggttcatatctgtgttgctgactacagtgtctgtgatgtccgcatgtccaggcctgtttggggttgcctagcgactcttctggcacagggtgtgtctgtggtatacctgtgaggtggttgacaattagtagtttaatcacagggtgtgtgtgtgtgtgtgtgtgtgtgtgtttatgtgcacgcatgtatatgcatcaccacgtagccaggaggggcctgttggggtttgagtcactgggatcttcctggtgagaggtaagagaagtcactgggcttagctgggcctctgaggcctgtatggaactcttggttgctgaggcaaccatggacctgttgctaggagatagctggggaaggcccaaggccgcccagggcagagagaggagacgaagagtttgggacagtgggggaggagatgggaagggatgggatttctgggtcccagagcgggtgggatactcacgcacagcttcttcactggtggggggtggggcacacattatttctcactggtcatgatttacaagaagaaaaataaaactgcttttggaaccacaaaaaaaaaaaaaaaaaaaaaaaaaaaa

SEQ ID NO: 49 - DNA, 1853 bp, Homo sapiens
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacacttaaaaaaaaaaaaaaaaaaaaaaaaaaaaaaaaaaaaaaaaaaaaaaaaaaaaaaaaaaaaaaaa

SEQ ID NO: 50 - DNA, 1967 bp, Homo sapiens
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacacttaaaaaaaaaaaaaaaaaaaaaaaaaaaaaaaaaaaaaaaaaaaaaaaaaaaaaaaaaaaaaaaa

SEQ ID NO: 51 - DNA, 1668 bp, Homo sapiens
acgggccaaggcggcgcgtctcgggggtggagcctggaggtgaccgcgccgctgcaacgcccccaccccccgcggtcgcagtggttcagcccgagaacttttcattcataaaaagaaaagactccgcacggcgcgggtgagtcagaacccagcagccgtgtaccccgcagagccgccagccccgggcatgttccgagacttcggggaacccggcccgagctccgggaacggcggcgggtacggcggccccgcgcagcccccggccgcagcgcaggcagcccagcagaagttccacctggtgccaagcatcaacaccatgagtggcagtcaggagctgcagtggatggtacagcctcatttcctggggcccagcagttaccccaggcctctgacctaccctcagtacagccccccacaaccccggccaggagtcatccgggccctggggccgcctccaggggtacgtcgaaggccttgtgaacagatcagcccggaggaagaggagcgccgccgagtaaggcgcgagcggaacaagctggctgcggccaagtgcaggaaccggaggaaggaactgaccgacttcctgcaggcggagactgacaaactggaagatgagaaatctgggctgcagcgagagattgaggagctgcagaagcagaaggagcgcctagagctggtgctggaagcccaccgacccatctgcaaaatcccggaaggagccaaggagggggacacaggcagtaccagtggcaccagcagcccaccagccccctgccgccctgtaccttgtatctccctttccccagggcctgtgcttgaacctgaggcactgcacacccccacactcatgaccacaccctccctaactcctttcacccccagcctggtcttcacctaccccagcactcctgagccttgtgcctcagctcatcgcaagagtagcagcagcagcggagacccatcctctgacccccttggctctccaaccctcctcgctttgtgaggcgcctgagccctactccctgcagatgccaccctagccaatgtctcctccccttcccccaccggtccagctggcctggacagtatcccacatccaactccagcaacttcttctccatccctctaatgagactgaccatattgtgcttcacagtagagccagcttggggccaccaaagctgcccactgtttctcttgagctggcctctctagcacaatttgcactaaatcagagacaaaatatttcccatttgtgccagaggaatcctggcagcccagagactttgtagatccttagaggtcctctggagccctaaccccttccagatcactgccacactctccatcaccctcttcctgtgatccacccaaccctatctcctgacagaaggtgccactttacccacctagaacactaactcaccagccccactgccagcagcagcaggtgattggaccaggccattctgccgccccctcctgaaccgcacagctcaggaggcgcccttggcttctgtgatgagctgatctgcggatctcagctttgagaagccttcagctccagggaatccaagcctccacagcgagggcagctgctatttattttcctaaagagagtatttttatacaaacctaccaaaatggaataaaaggcttgaagctgtg

SEQ ID NO: 52 - DNA, 1762 bp, Homo sapiens
cggccgagggcggggcagggaggcagcatgctaaaccgggtgcgctcggccgtggcgcacctggtgagctccgggggcgctccgcctccgcgccccaaatccccggacctgcccaacgccgcctcggcgccgcccgccgccgctccagaagcgcccaggagccctcccgcgaaggctgggagcgggagcgcgacgcccgcgaaggctgttgaggctcgagcgagcttctccagaccgacctttctgcagctgagccccggggggctgcgacgcgccgatgaccacgcgggccgggctgtgcaaagccccccggacacgggccgccgcctgccctggagcacaggctacgccgaggtcatcaatgctggcaagagtcggcacaatgaggaccaggcttgctgtgaagtggtgtatgtggaaggtcggaggagtgttacaggagtacctagggagcctagccgaggccagggactctgcttctactactggggcctatttgatgggcatgcagggggcggagctgctgaaatggcctcacggctcctgcatcgccatatccgagagcagctaaaggacctggtagagatacttcaggacccttcgccaccacccctctgcctcccaaccactccggggaccccagattcctccgatccctctcacttgcttggccctcagtcctgctggtcttcacagaaggaagtgagccacgagagcctggtagtgggggccgttgagaatgccttccagctcatggatgagcagatggcccgggagcggcgtggccaccaagtggaggggggctgctgtgcactggttgtgatctacctgctaggcaaggtgtacgtggccaatgcaggcgatagcagggccatcattgtccggaatggtgaaatcattccaatgtcccgggagtttaccccggagactgagcgccagcgtcttcagctgcttggcttcctgaaaccagagctgctaggcagtgaattcacccaccttgagttcccccgcagagttctgcccaaggagctggggcagaggatgttgtaccgggaccagaacatgaccggctgggcctacaaaaagatcgagctggaggatctcaggtttcctctggtctgtggggagggcaaaaaggctcgggtgatggccaccattggggtgacccgaggcttgggagaccacagccttaaggtctgcagttccaccctgcccatcaagccctttctctcctgcttccctgaggtacgagtgtatgacctgacacaatatgagcactgcccagatgatgtgctagtcctgggaacagatggcctgtgggatgtcactactgactgtgaggtagctgccactgtggacagggtgctgtcggcctatgagcctaatgaccacagcaggtatacagctctggcccaagctctggtcctgggggcccggggtaccccccgagaccgtggctggcgtctccccaacaacaagctgggttccggggatgacatctctgtcttcgtcatccccctgggagggccaggcagttactcctgaggggctgaacaccatccctcccactagcctctccatacttactcctctcacagcccaaattctgaagttgtctccctgacccttctttagtggcaacttaactgaagaagggatgtccgctatatccaaaattacagctattggcaaataaacgagatggataaaggtgaaaaaaaaaaaaaaaaaaaaaaaaaaaaaaaaaaaaaaaaaaaaaaaa

SEQ ID NO: 53 - DNA, 1125 bp, Homo sapiens
ctctctttcactgcaaggcggcggcaggagaggttgtggtgctagtttctctaagccatccagtgccatcctcgtcgctgcagcgacacacgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacctccctggcctggtctgctgatggccagactctgtttgctggctacacggacaacctggtgcgagtgtggcaggtgaccattggcacacgctagaagtttatggcagagctttacaaataaaaaaaaaactggcttttctgacaaaaaaaaaaaaaaaa

SEQ ID NO: 54 - DNA, 987 bp, Homo sapiens
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 55 - DNA, 609 bp, Homo sapiens
ttctcttcctgctctccatcatggcgcaggatcaaggtgaaaaggagaaccccatgcgggaacttcgcatccgcaaactctgtctcaacatctgtgttggggagagtggagacagactgacgcgagcagccaaggtgttggagcagctcacagggcagacccctgtgttttccaaagctagatacactgtcagatcctttggcatccggagaaatgaaaagattgctgtccactgcacagttcgaggggccaaggcagaagaaatcttggagaagggtctaaaggtgcgggagtatgagttaagaaaaaacaacttctcagatactggaaactttggttttgggatccaggaacacatcgatctgggtatcaaatatgacccaagcattggtatctacggcctggacttctatgtggtgctgggtaggccaggtttcagcatcgcagacaagaagcgcaggacaggctgcattggggccaaacacagaatcagcaaagaggaggccatgcgctggttccagcagaagtatgatgggatcatccttcctggcaaataaattcccgtttctatccaaaagagcaataaaaagttttcagtgaaatgtgcaa

SEQ ID NO: 56 - DNA, 579 bp, Homo sapiens
tctttcttttcgccatcttttgtctttccgtggagctgtcgccatgaaggtcgagctgtgcagttttagcgggtacaagatctaccccggacacgggaggcgctacgccaggaccgacgggaaggttttccagtttcttaatgcgaaatgcgagtcggctttcctttccaagaggaatcctcggcagataaactggactgtcctctacagaaggaagcacaaaaagggacagtcggaagaaattcaaaagaaaagaacccgccgagcagtcaaattccagagggccattactggtgcatctcttgctgatataatggccaagaggaatcagaaacctgaagttagaaaggctcaacgagaacaagctatcagggctgctaaggaagcaaaaaaggctaagcaagcatctaaaaagactgcaatggctgctgctaaggcacctacaaaggcagcacctaagcaaaagattgtgaagcctgtgaaagtttcagctccccgagttggtggaaaacgctaaactggcagattagatttttaaataaagattggattataactctagaaaaaaaaaaaaaaaaa

SEQ ID NO: 57 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa